(19) United States
(12) Patent Application Publication
(10) Pub. No.: US 2018/0148794 A1
(43) Pub. Date: May 31, 2018

(54) METHODS OF PROSTATE CANCER PROGNOSIS

(71) Applicant: KONINKLIJKE PHILIPS N.V., EINDHOVEN (NL)

(72) Inventors: RALF HOFFMANN, EINDHOVEN (NL); EVELINE DEN BIEZEN-TIMMERMANS, EINDHOVEN (NL); DIANNE ARNOLDINA MARGARETHA WILHELMINA VAN STRIJP, EINDHOVEN (NL); ANNE GODEFRIDA CATHARINA VAN BRUSSEL, EINDHOVEN (NL); MARCIA ALVES DE INDA, EINDHOVEN (NL); JANNEKE WROBEL, EINDHOVEN (NL)

(21) Appl. No.: 15/577,480

(22) PCT Filed: May 26, 2016
(86) PCT No.: PCT/EP2016/061885

§ 371 Date: Nov. 28, 2017

(30) Foreign Application Priority Data
May 29, 2015 (EP) 15169782.8

(51) Int. Cl. C12Q 1/6886

(52) U.S. Cl. CPC C12Q 1/6886; C12Q 2600/166; C12Q 2600/158; C12Q 2600/118

(57) ABSTRACT

The present invention pertains to methods for determining gene expression signatures for diagnosing patients with prostate cancer, e.g., to establish a prognosis for such patients, for determining the predisposition of said patient to develop aggressive or indolent disease, for example after primary treatment, and/or for identification and stratification of patients for available therapies. The invention also relates to products used in such methods, e.g. kits, software, and the like. The present invention further relates to therapies for use in the treatment of patients that have been diagnosed, identified, or stratified in any of the methods dis closed herein.

### FIELD OF THE INVENTION

The invention pertains to methods for determining gene expression signatures for diagnosing patients with prostate cancer, e.g., to establish a prognosis for such patients, for determining the predisposition of said patient to develop aggressive or indolent disease, for example after primary treatment, and/or for identification and stratification of patients for available therapies. The invention also relates to products used in such methods, e.g. kits, software, and the like. The invention further relates to therapies for use in the treatment of patients that have been diagnosed, identified, or stratified in any of the methods disclosed herein.

### BACKGROUND OF THE INVENTION

Cancer is a class of diseases in which a group of cells display uncontrolled growth, invasion and sometimes metastasis. These three malignant properties of cancers differentiate them from benign tumors, which are self-limited, do not invade or metastasize. Prostate Cancer (PCa) is the most commonly occurring non-skin malignancy in men, with estimated 900,000 new cases diagnosed world-wide in 2008. Due to ageing populations, the incidence of PCa will dramatically increase in the coming years. Routine diagnosis by determination of blood levels of the prostate-specific antigen (PSA), digital rectal exam (DRE) and transrectal ultrasound analysis (TRUS) leads to significant first-line over-diagnosis of non-cancerous, benign prostate conditions: of the approx. 1 million prostate biopsies annually performed in the U.S. alone to find about 250,000 new cases, about 75% are done unnecessarily, incurring both substantial complications (urosepsis, bleedings, urinary retention) in patients and total cost of >US$ 2 billion (˜US$ 2,100 per biopsy procedure). At least 4 out of 100 men with a negative biopsy are likely to be hospitalized due to side-effects and 9 out of 10,000 biopsied patients are at risk of dying from the currently used procedure.

Of the approximately 250,000 newly detected PCa cases in the U.S. per year, about 200,000 are initially characterized as localized disease, i.e. as cancer confined to the prostate organ. This condition is to a certain extent curable by primary treatment approaches, such as radiation therapy or, in particular, the partial or total removal of the prostate by surgery (prostatectomy). However, these interventions typically come with serious side effects, particularly urinary incontinence and/or erectile dysfunctions as very frequent consequences of prostatectomy. Up to 50% of men undergoing radical prostatectomy develop urinary incontinence. Studies have shown that, one year after surgery, between 15% and 50% of men still report such problems. Erection problems likewise are serious side effects of radical prostatectomy (RP). Only about half of the operated men are able to regain some of their ability to have erections. Furthermore, all routinely applied treatments for localized PCa are expensive (typically in the order of US$ 20-30,000) and incur total direct costs of US$ 5 billion in the U.S. each year.

Among the ˜200,000 men in the United States with clinically localized disease at diagnosis, up to 50% have very-low- or low-risk cancer. Accordingly, the NCCN (National Comprehensive Cancer Network) recently revised their PCa treatment guidelines to expand active surveillance (AS) as a gentle and convenient treatment alternative for patients with such low risk disease. By referring appropriate patients to AS, the quality of life for such patients is significantly improved as compared with men having undergone primary treatment and the 5-year cost for AS is reported to be less than US$ 10,000 per patient.

Moreover, in case surgery (vs. AS) is selected as the treatment of choice for a given patient, it is of significant advantage to stratify for the extent of surgery according to the potential aggressiveness of the patient's tumor. For instance, nerve-sparing operation techniques could be more generally applied for men with predicted low-risk disease to minimize potency-related adverse effects of radical prostatectomy. Likewise, according to the European Association Of Urology (EAU)'s latest Prostate Cancer Guidelines, extended lymph node dissection is recommended in case of a predicted high-risk cancer despite the fact that the procedure is complex, time-consuming and associated with higher complication rates as compared with more limited procedures. Consequently, while less limited lymph node dissection has shown to miss about 50% of lymph node metastases, the management of men with localized prostate cancer requires highly accurate pre-surgical predictions of the aggressiveness potential of an individual tumor to provide most optimal care for each patient.

US 2013/196321 A1 describes molecular assays, which comprise measurement of expression levels of one or more genes in a sample from a prostate cancer patient to provide information concerning the likelihood of a clinical outcome, identify risk classification and facilitate treatment decision making. The reference discloses a number of genes to be analyzed in such assays, which are normalized to e.g. a housekeeping gene. Furthermore, kits comprising gene-specific probes and/or primers to perform the assays and computer-based systems, algorithms and computer program products to perform the analysis of the expression levels are also disclosed.

The article “DYRK2 controls the epithelial-mesenchymal transition in breast cancer by degrading Snail” by Mimoto Rei et al. in Cancer Letters (2013), Volume 339, Number 2, pages 214-225, discloses that DYRK2 is down regulated in human breast cancer tissue and that patients with low DYRK2-expressing tumors have a worse outcome than those with high SYRK2-expressing tumors. The authors conclude that DYRK2 is a potential prognostic marker for breast cancer and additionally mention, that it has been demonstrated that DYRK2 has also been found to be down-regulated in lung, colon and prostate cancers.

WO 2010/131194 A1 relates to PDE4D7 as a marker for malignant hormone sensitive prostate cancer and discloses a method for diagnosing, detecting, monitoring and prognosticating malignant, hormone sensitive prostate cancer comprising the step of determining the level of PDE4D7. In addition, methods for identifying an individual for eligibility for certain cancer therapy are described as well as a composition for carrying out the method, comprising oligonucleotides or probes specific for the PDE4D7 expression product.

WO 2014/153442 A2 discloses methods for diagnosis, prognosis and treatment of ovarian cancer which involve the determination of the expression levels of certain marker genes. The expression levels are converted into an index, wherein the genes are weighted equally with the expression levels of down-regulated genes subtracted from the ones of up-regulated genes.

### SUMMARY OF THE INVENTION

The present invention relates to the identification and use of gene expression profiles, signatures, or patterns of biomarker genes of interest (also referred to as marker genes) with clinical relevance to prostate cancer. In particular, the invention is based on the gene expression analysis of nucleic acids, preferably transcripts of biomarker genes, obtained from biological samples and the identification of biomarker genes that are correlated with aggressive and indolent disease, patient survival and prostate cancer recurrence. Expression analysis of these marker genes can be used in providing a Prostate Cancer Progression Index (PCPI) that can be used in the classification of patients into those with an aggressive and indolent disease and with corresponding poor or good prognosis.

Further, the PCPI can also be used to predict the progression of disease in previously clinically diagnosed and treated subjects and to stratify the patients according to their individual PCPI. Therefore, the PCPI provides a very helpful parameter for personalized medicine relating to the diagnosis, prognosis and treatment of prostate cancer patients. The PCPI may be used alone or in combination with other means and methods that provide information on the patients' personal disease status or disease stage.

Physicians and/or pathologists can advantageously use the PCPI to confirm results obtained in other methods for diagnosing, identifying, prognosticating, classifying, and/or stratifying patients, e.g. patients with a predisposition to develop aggressive versus indolent prostate cancer forms. The methods and means provided by the invention therefore help establish better diagnosis, prognosis, etc. to find the best treatment for a patient, and to avoid unnecessary surgery or other treatments that are dangerous due to side-effects, occasionally superfluous and result in enormous costs for the public health systems.

One aspect of the invention is directed to a method comprising:

determining a gene expression level for the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, to obtain a subject expression profile for a subject.

In some embodiments, the method further comprises:

classifying the subject as prostate cancer-positive or prostate cancer-negative based on the subject expression profile; and/or

classifying the subject as having a good prognosis or a poor prognosis of prostate cancer based on the subject expression profile, wherein the good prognosis predicts an increased likelihood of survival within a predetermined period after initial diagnosis and/or no progression of disease after primary treatment, and the poor prognosis predicts an aggressive disease, a decreased likelihood of survival, an increased likelihood of biochemical recurrence, clinical recurrence, and/or the presence of local or distant metastases, within a predetermined period after initial diagnosis; and/or

classifying the subject as having or not having a predisposition of prostate cancer that is susceptible to disease progression based on the subject expression profile, wherein the predisposition predicts an aggressive disease, a decreased likelihood of survival, an increased likelihood of biochemical recurrence, clinical recurrence, and/or the presence of local or distant metastases, within a predetermined period after initial diagnosis.

In one embodiment, the method further comprises:

classifying the subject as prostate cancer-positive or prostate cancer-negative based on the subject expression profile; or

classifying the subject as having a good prognosis or a poor prognosis of prostate cancer based on the subject expression profile, wherein the good prognosis predicts an increased likelihood of survival within a predetermined period after initial diagnosis and/or no progression of disease after primary treatment, and the poor prognosis predicts an aggressive disease, a decreased likelihood of survival, an increased likelihood of biochemical recurrence, clinical recurrence, and/or the presence of local or distant metastases, within a predetermined period after initial diagnosis; or

classifying the subject as having or not having a predisposition of prostate cancer that is susceptible to disease progression based on the subject expression profile, wherein the predisposition predicts an aggressive disease, a decreased likelihood of survival, an increased likelihood of biochemical recurrence, clinical recurrence, and/or the presence of local or distant metastases, within a predetermined period after initial diagnosis.

In one embodiment, the method further comprises:

classifying the subject as prostate cancer-positive or prostate cancer-negative based on the subject expression profile; or

classifying the subject as having a good prognosis or a poor prognosis of prostate cancer based on the subject expression profile, wherein the good prognosis predicts an increased likelihood of survival within a predetermined period after initial diagnosis and/or no progression of disease after primary treatment, and the poor prognosis predicts an aggressive disease, a decreased likelihood of survival, an increased likelihood of biochemical recurrence, clinical recurrence, and/or the presence of local or distant metastases, within a predetermined period after initial diagnosis; and

classifying the subject as having or not having a predisposition of prostate cancer that is susceptible to disease progression based on the subject expression profile, wherein the predisposition predicts an aggressive disease, a decreased likelihood of survival, an increased likelihood of biochemical recurrence, clinical recurrence, and/or the presence of local or distant metastases, within a predetermined period after initial diagnosis. In one embodiment, the method further comprises:

classifying the subject as prostate cancer-positive or prostate cancer-negative based on the subject expression profile; and

classifying the subject as having a good prognosis or a poor prognosis of prostate cancer based on the subject expression profile, wherein the good prognosis predicts an increased likelihood of survival within a predetermined period after initial diagnosis and/or no progression of disease after primary treatment, and the poor prognosis predicts an aggressive disease, a decreased likelihood of survival, an increased likelihood of biochemical recurrence, clinical recurrence, and/or the presence of local or distant metastases, within a predetermined period after initial diagnosis; or

classifying the subject as having or not having a predisposition of prostate cancer that is susceptible to disease progression based on the subject expression profile, wherein the predisposition predicts an aggressive disease, a decreased likelihood of survival, an increased likelihood of biochemical recurrence, clinical recurrence, and/or the presence of local or distant metastases, within a predetermined period after initial diagnosis.

In one embodiment, the method further comprises:

classifying the subject as prostate cancer-positive or prostate cancer-negative based on the subject expression profile; and

classifying the subject as having a good prognosis or a poor prognosis of prostate cancer based on the subject expression profile, wherein the good prognosis predicts an increased likelihood of survival within a predetermined period after initial diagnosis and/or no progression of disease after primary treatment, and the poor prognosis predicts an aggressive disease, a decreased likelihood of survival, an increased likelihood of biochemical recurrence, clinical recurrence, and/or the presence of local or distant metastases, within a predetermined period after initial diagnosis; and

classifying the subject as having or not having a predisposition of prostate cancer that is susceptible to disease progression based on the subject expression profile, wherein the predisposition predicts an aggressive disease, a decreased likelihood of survival, an increased likelihood of biochemical recurrence, clinical recurrence, and/or the presence of local or distant metastases, within a predetermined period after initial diagnosis.

Another aspect of the invention is directed to a method comprising:

classifying a subject as prostate cancer-positive or prostate cancer-negative based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7; and/or

classifying a subject as having a good prognosis or a poor prognosis based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, and wherein a good prognosis predicts an increased likelihood of survival within a predetermined period after initial diagnosis and/or no progression of disease after primary treatment, and poor prognosis predicts an aggressive disease, a decreased likelihood of survival, an increased likelihood of biochemical recurrence, clinical recurrence, and/or the presence of local or distant metastases, within a predetermined period after initial diagnosis; and/or

classifying a subject as having or not having a predisposition of prostate cancer that is susceptible to disease progression based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, wherein the predisposition predicts an aggressive disease, a decreased likelihood of survival, an increased likelihood of biochemical recurrence, clinical recurrence, and/or the presence of local or distant metastases, within a predetermined period after initial diagnosis.

In one embodiment, a method comprises:

classifying a subject as prostate cancer-positive or prostate cancer-negative based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7; or

classifying a subject as having a good prognosis or a poor prognosis based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, and wherein a good prognosis predicts an increased likelihood of survival within a predetermined period after initial diagnosis and/or no progression of disease after primary treatment, and poor prognosis predicts an aggressive disease, a decreased likelihood of survival, an increased likelihood of biochemical recurrence, clinical recurrence, and/or the presence of local or distant metastases, within a predetermined period after initial diagnosis; or

classifying a subject as having or not having a predisposition of prostate cancer that is susceptible to disease progression based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, wherein the predisposition predicts an aggressive disease, a decreased likelihood of survival, an increased likelihood of biochemical recurrence, clinical recurrence, and/or the presence of local or distant metastases, within a predetermined period after initial diagnosis.

In one embodiment, a method comprises:

classifying a subject as prostate cancer-positive or prostate cancer-negative based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7; or

classifying a subject as having a good prognosis or a poor prognosis based on a subject expression profile of the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, and wherein a good prognosis predicts an increased likelihood of survival within a predetermined period after initial diagnosis and/or no progression of disease after primary treatment, and poor prognosis predicts an aggressive disease, a decreased likelihood of survival, an increased likelihood of biochemical recurrence, clinical recurrence, and/or the presence of local or distant metastases, within a predetermined period after initial diagnosis; and

classifying a subject as having or not having a predisposition of prostate cancer that is susceptible to disease progression based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, wherein the predisposition predicts an aggressive disease, a decreased likelihood of survival, an increased likelihood of biochemical recurrence, clinical recurrence, and/or the presence of local or distant metastases, within a predetermined period after initial diagnosis.

In one embodiment, a method comprises:

classifying a subject as prostate cancer-positive or prostate cancer-negative based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7; and

classifying a subject as having a good prognosis or a poor prognosis based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, and wherein a good prognosis predicts an increased likelihood of survival within a predetermined period after initial diagnosis and/or no progression of disease after primary treatment, and poor prognosis predicts an aggressive disease, a decreased likelihood of survival, an increased likelihood of biochemical recurrence, clinical recurrence, and/or the presence of local or distant metastases, within a predetermined period after initial diagnosis; or

classifying a subject as having or not having a predisposition of prostate cancer that is susceptible to disease progression based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, wherein the predisposition predicts an aggressive disease, a decreased likelihood of survival, an increased likelihood of biochemical recurrence, clinical recurrence, and/or the presence of local or distant metastases, within a predetermined period after initial diagnosis.

In one embodiment, a method comprises:

classifying a subject as prostate cancer-positive or prostate cancer-negative based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7; and

classifying a subject as having a good prognosis or a poor prognosis based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, and wherein a good prognosis predicts an increased likelihood of survival within a predetermined period after initial diagnosis and/or no progression of disease after primary treatment, and poor prognosis predicts an aggressive disease, a decreased likelihood of survival, an increased likelihood of biochemical recurrence, clinical recurrence, and/or the presence of local or distant metastases, within a predetermined period after initial diagnosis; and

classifying a subject as having or not having a predisposition of prostate cancer that is susceptible to disease progression based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, wherein the predisposition predicts an aggressive disease, a decreased likelihood of survival, an increased likelihood of biochemical recurrence, clinical recurrence, and/or the presence of local or distant metastases, within a predetermined period after initial diagnosis.

In some embodiments of various aspects of the invention, the subject is a patient diagnosed with prostate cancer.

In some embodiments of various aspects of the invention, the subject expression profile is obtained from a sample from a subject.

In a particular embodiment of various aspects of the invention, the sample is cell lines.

In some embodiments of various aspects of the invention, the patient is selected from the group comprising (a) those with newly diagnosed prostate cancer, (b) those that have been subjected to primary treatment for prostate cancer.

In an embodiment of various aspects of the invention, the patient was previously diagnosed with clinically localized prostate cancer.

In an embodiment of various aspects of the invention, the primary treatment is selected from the group consisting of prostate surgery, prostate removal, radiotherapy, chemotherapy, limited or extended lymph node removal.

In an embodiment of various aspects of the invention, the patient in group (a) are further selected from patients with newly diagnosed prostate cancer that have been subjected to at least one of the following methods: determination of the blood levels of prostate-specific antigen (PSA), digital rectal exam (DRE) and transrectal ultrasound analysis (TRUS), and biopsy of the prostate.

In some embodiments of various aspects of the invention, the predetermined period is at least 6 months, at least 1 year, at least 2 years, at least 3 years, at least 4 years, at least 5 years, at least 10 years, or at least 15 years.

In some embodiments of various aspects of the invention, the gene expression level is determined by detecting mRNA expression using one or more probes and/or one or more probe sets.

In some embodiments of various aspects of the invention, the gene expression level is determined by an amplification based method and/or microarray analysis.

In some embodiments of various aspects of the invention, the gene expression level is determined by RNA sequencing, PCR, qPCR or multiplex-PCR.

In some embodiments of various aspects of the invention, the patient expression profile is converted to a prostate cancer progression index (PCPI).

In an embodiment, the prostate cancer progression index is calculated according to the following equation:

PCPI=(GEV_av_GOI_up)−(GEV_av_GOI_down),

wherein GEV_av_GOI_up is an average gene expression value of up-regulated genes, and wherein GEV_av_GOI_down is an average gene expression value of down-regulated genes, wherein the values are determined on the basis of normalized gene expression data per each subject sample. The PCPI calculation can render a good prediction of prognosis or a good performance in other clinical applications as described herein.

In some embodiments of various aspects of the invention, the expression level of the set of the signature genes is normalized to the expression of a reference gene, preferably wherein the reference gene is a housekeeping gene, and more preferably wherein the housekeeping gene is TBP, HPRT1, ACTB, RPLP0, PUM1, POLR2A or B2M.

In a particular embodiment of the invention, the subject is classified as prostate cancer-positive when expression of any one or more genes selected from the group consisting of COL1A1, COL3A1, COL5A2, INHBA, THBS2, VCAN, BGN, BIRC5, DYRK2 is up-regulated as compared with the corresponding gene expression levels in a control sample, and wherein the expression of any one or more genes selected from the group consisting of AZGP1, FBLN1, ILK, KRT15, MEIS2, MYBPC1, PAGE4, SRD5A2 and optionally of PDE4D5 and/or PDE4D7 is down-regulated as compared with the corresponding gene expression levels in a control sample; or the subject is classified as prostate cancer-negative when expression of any one or more genes selected from the group consisting of COL1A1, COL3A1, COL5A2, INHBA, THBS2, VCAN, BGN, BIRC5, DYRK2 is down-regulated as compared with the corresponding gene expression levels in a control sample, and wherein the expression of any one or more genes selected from the group consisting of AZGP1, FBLN1, ILK, KRT15, MEIS2, MYBPC1, PAGE4, SRD5A2 and optionally of PDE4D5 and/or PDE4D7 is up-regulated as compared with the corresponding gene expression levels in a control sample. The selected genes can render a good performance in the classification.

In some embodiments of various aspects of the invention, the subject is classified as having a good prognosis if the prostate cancer progression index is below a selected threshold or as having a poor prognosis if the prostate cancer progression index is above the selected threshold; and/or the subject is classified as having a predisposition of prostate cancer that is susceptible to disease progression if the prostate cancer progression index is above a selected threshold or as not having a predisposition of prostate cancer that is susceptible to disease progression if the prostate cancer progression index is below the selected threshold.

In some embodiments of various aspects of the invention, the method further comprises stratifying the subject for the risk of aggressive disease versus non-aggressive disease according to the prognosis determined or the predisposition determined; and/or providing a suitable cancer treatment to the subject in need thereof according to the prognosis determined or the predisposition determined.

In some embodiments of various aspects of the invention, the cancer treatment is selected from prostate surgery, prostate removal, radiotherapy, chemotherapy, hormone therapy, limited or extended lymph node removal or combinations thereof, preferably the combination of hormone therapy and radiation therapy.

In some embodiments of various aspects of the invention, the poor prognosis or the predisposition of prostate cancer that is susceptible to disease progression is indicative of eligibility of the subject to be treated with any one or more of the treatments selected from the group consisting of prostate surgery, prostate removal, chemotherapy, radiotherapy, limited or extended lymph node dissection.

In some embodiments of various aspects of the invention, a predisposition for aggressive prostate cancer is indicative of eligibility for a treatment selected from the group comprising re-biopsy, prostate surgery, prostate removal, chemotherapy, radiotherapy, hormone therapy limited or extended lymph node dissection or combinations thereof, preferably the combination of hormone therapy and radiation therapy.

In some embodiments of various aspects of the invention, the method further comprises displaying or outputting a result of one of the steps to a user interface device, a computer readable storage medium, a monitor, or a computer that is part of a network.

A further aspect relates to a product comprising:

primers and/or probes for determining the gene expression level for the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7;

optionally further comprising primers and/or probes for determining the expression levels of a set of genes listed in either FIG. 1 or 2 other than the above-mentioned genes; and/or

optionally further comprising primers and/or probes for determining the gene expression level of a reference gene, preferably wherein the reference gene is a housekeeping gene, and more preferably wherein the housekeeping gene is TBP, HPRT1, ACTB, RPLP0, PUM1, POLR2A or B2M.

In some embodiments, the product is a kit including for example a PCR kit, a RNA-sequencing kit, or a microarray kit preferably a DNA microarray kit. In another embodiment, the product is a microarray. The product can provide an efficient way for determining the levels as described herein and thus can render a good performance in clinical applications as described herein.

In some embodiments, the product is a product for performing the method of the invention, or the product is a product for diagnosis of prostate cancer, for establishing a prognosis for a patient diagnosed with prostate, for stratification of a patient diagnosed with prostate cancer, for the determination of predisposition for aggressive or indolent prostate cancer in a patient having a prostate cancer.

In some embodiments, the product is a composition comprising a set of nucleic acid molecules each comprising at least one polynucleotide probe sequence in the analysis of the gene expression of the genes comprised by any one of FIG. 1 or 2, or the genes as described above.

In some embodiments, the product is an nucleic acid array comprising for each gene in a set of genes, the set of genes comprising the genes listed in FIG. 1 or 2, or the genes as described above, one or more polynucleotide probes complementary and hybridizable to a coding sequence in the gene, for determining a prostate expression profile and/or a PCPI as defined above.

In some embodiments, the product is a kit for diagnosis of prostate cancer, for establishing a prognosis for a patient diagnosed with prostate, for stratification of a patient diagnosed with prostate cancer, for the determination of predisposition for aggressive or indolent prostate cancer in a patient having a prostate cancer according any one of methods as described herein comprising: a) an array as defined herein, b) a kit control; and c) optionally instructions for use.

In some embodiments, the product is a nucleic acid array for the expression analysis of nucleic acids obtained from a biological sample, wherein said array comprises a substrate comprising probes suitable for the specific binding of nucleic acids or fragments thereof encoded by the genes referred to in FIG. 1 or 2, or the genes as described above.

In some embodiments, the product is an article for PCR comprising reagents for specifically amplifying and detecting nucleic acids or fragments thereof encoded by the genes referred to of FIG. 1 or 2, or the genes as described above.

A further aspect of the invention relates to a therapy selected from prostate surgery, prostate removal, radiotherapy, hormone therapy, chemotherapy, limited or extended lymph node removal or combinations thereof for use in the treatment of patients identified as eligible, wherein said patients are diagnosed with a predisposition for aggressive prostate cancer as defined the methods of the invention.

A further aspect of the invention relates to a computer program product, comprising computer readable code stored on a computer readable medium or downloadable from a communications network, which, when run on a computer, implement one or more steps or all the steps of any one of the methods as described herein.

A further aspect of the invention relates to a non-transitory computer readable storage medium with an executable program stored thereon, wherein the program instructs a microprocessor to perform one or more of the steps of any of the methods of the invention.

A yet further aspect of the invention relates to a device for gene expression analysis of nucleic acids (or fragments thereof) encoded by genes referred to in FIG. 1 or 2, or the genes described above, wherein the device preferably comprises:

a) a database including records comprising reference gene expression values associated with clinical outcomes, each reference profile comprising the expression levels of a set of genes listed in either FIG. 1 or 2, or the genes as described above, and/or

b) a user interface capable of receiving and/or inputting a selection of gene expression values of a set of genes, the set comprising genes listed in FIG. 1 or 2, or the genes as described above, for use in comparing to the gene reference expression profiles in the database;

c) an output that displays a prediction of clinical prognosis according to the expression levels of the set of genes.

A yet further aspect of the invention relates to a device for performing the method of the invention.

A still further aspect of the invention relates to a system comprising the product of invention and the computer program product or the non-transitory computer readable storage medium of the invention or the device for gene expression analysis of nucleic acids of the invention or a device for performing the method of the invention.

### BRIEF DESCRIPTION OF THE DRAWINGS

FIG. 1 shows a table comprising gene candidates analyzed which are derived from publically available study data.

FIG. 2 shows a table comprising 57 gene candidates analyzed to build the PCPI.

FIG. 3 shows an overview on the performance of PCPI_18 or PCPI_17 to predict the primary endpoint of development of distant metastases within 10-15 years after primary treatment (prostate surgery) for the data sets GSE21032, GSE41408, GSE25136, GSE16560, GSE10645.

FIG. 4 shows the performance of PCPI_18 to predict the primary endpoint of development of distant metastases within 10-15 years after primary treatment for the data set GSE46691.

FIG. 5 shows the performance of PCPI_18 or PCPI_17 to predict the primary endpoint of development of distant metastases within 10-15 years after primary treatment (prostate surgery in general) for the data sets GSE21032, GSE41408, GSE16560, GSE10645 in a multivariate COX regression analysis compared to standard clinical parameters like pre-treatment PSA, biopsy or pathology Gleason score, or clinical/pathology disease stage. The Chi Square as well as the Hazard Ratio for the PCPI and the individual clinical parameters is shown for each individual data set.

FIG. 6 shows an overview on the performance of PCPI_18 to predict the primary endpoint of development of biochemical disease recurrence (BCR) within 10-15 years after primary treatment (prostate surgery in general) for the data sets GSE21032, GSE41408, GSE25136.

FIG. 7 shows the performance of PCPI_18 to predict the primary endpoint of development of biochemical disease recurrence (BCR) within 10-15 years after primary treatment (prostate surgery in general) for the data sets GSE21032, GSE41408, GSE25136 in a multivariate COX regression analysis compared to standard clinical parameters like pre-treatment PSA, biopsy or pathology Gleason score, or clinical/pathology disease stage.

FIG. 8 shows a ROC curve cut-off analysis for PCPI_18 to support clinical decision making towards e.g. patient stratification for either active surveillance (AS) or active treatment (e.g., prostatectomy, radiotherapy and hormone therapy).

FIG. 9 lists genes of interest mentioned in this application with their Transcript ID, Protein ID as well as primer and probe sequences specific for the genes of interest.

FIG. 10 lists reference genes mentioned in this application with their Transcript ID, Protein ID as well as primer and probe sequences specific for the reference genes.

FIG. 11 shows an analysis of different recurrence risks over 5 to 10 years for 422 patients grouped according to the PCPI_19 and the NCCN clinical risk characteristics as can be inferred from the guidelines of the National Comprehensive Cancer Network's (NCCN) webpage (https://www.nccn.org/professionals/physician_gls/f_guidelines.asp).

FIG. 12 shows an analysis of different recurrence risks over 5 to 10 years for 407 patients grouped according to the GPSu score and the NCCN clinical risk characteristics as can be inferred from the guidelines of the National Comprehensive Cancer Network's (NCCN) webpage (https://www.nccn.org/professionals/physician_gls/f_guidelines.asp).

FIG. 13 shows an analysis of different recurrence risks over 5 to 10 years for 407 patients grouped according to the CCP score and the NCCN clinical risk characteristics as can be inferred from the guidelines of the National Comprehensive Cancer Network's (NCCN) webpage (https://www.nccn.org/professionals/physician gls/f guidelines.asp).

### DETAILED DESCRIPTION OF EMBODIMENTS

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first”, “second”, “third” or “(a)”, “(b)”, “(c)”, “(d)” etc. relate to steps of a method or use there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, proteins, bacteria, vectors, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

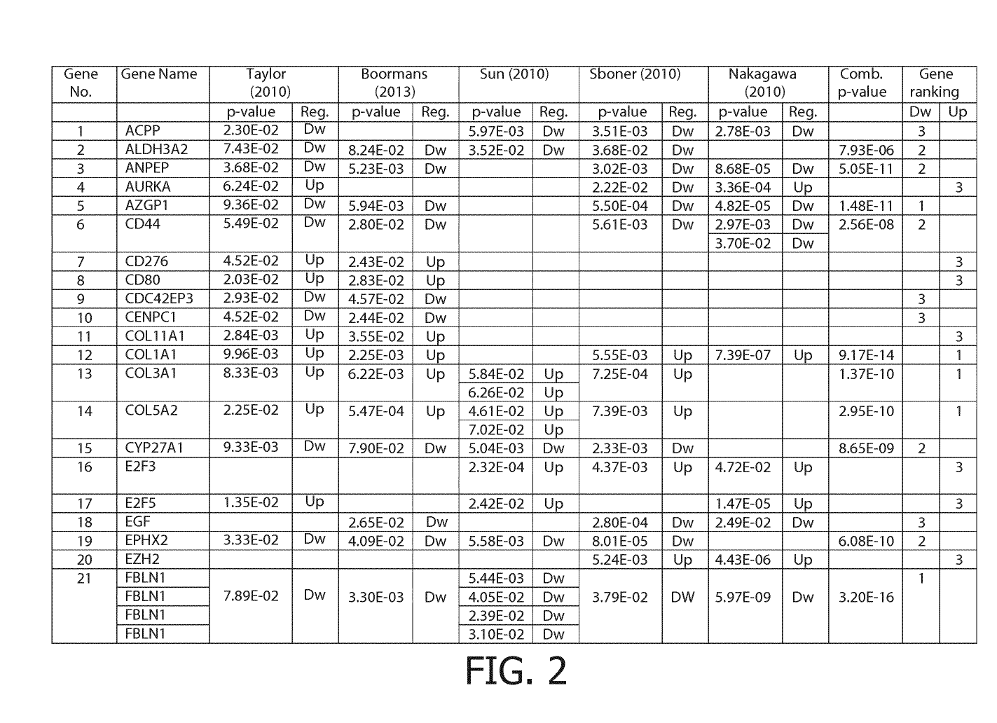

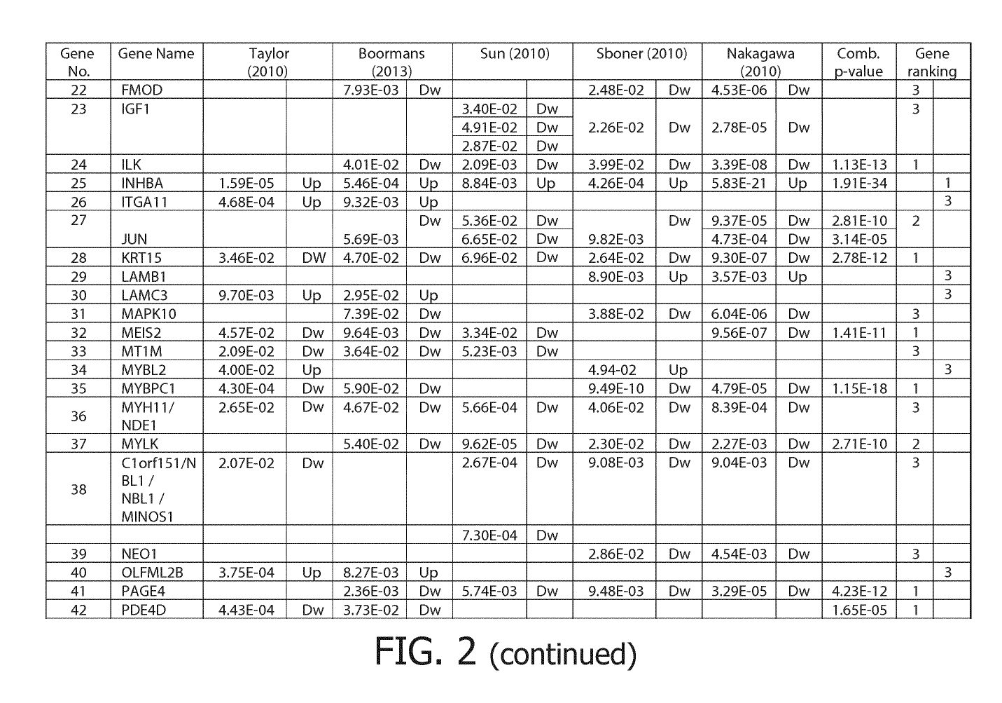

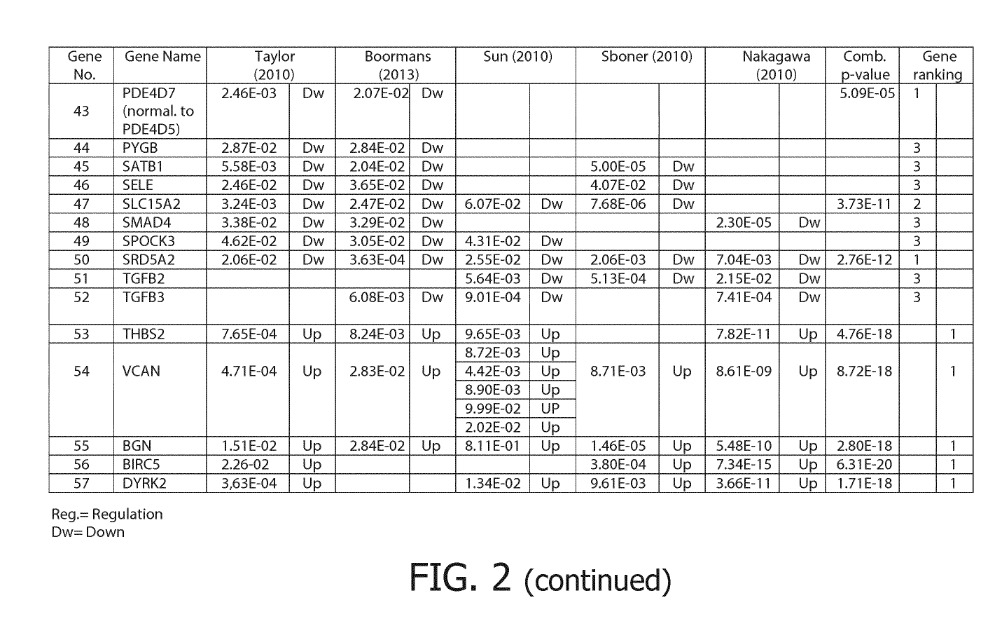

The term “signature gene(s)”, marker gene(s), or “gene of interest (GOI)” in the context of the present invention can be used interchangeably and designates biomarkers whose expression level is determined in the methods of the present invention. In preferred embodiments of the present invention, these genes are depicted in FIG. 2 of the present disclosure, and referred to explicitly below as well as in the claims. Therefore, the present invention concerns marker gene(s), the expression of which is either up-regulated or down-regulated when comparing the expression in prostate cancer tissue obtained after primary treatment or after initial prostate cancer diagnosis to the expression in prostate cancer tissue obtained before initial prostate cancer diagnosis and/or before initial prostate cancer-specific treatment. The marker gene expression in tissues obtained after primary treatment or after initial prostate cancer diagnosis may be correlated to patient outcome parameters, e.g. biochemical recurrence, development of metastasis, bone cancer and/or death.

When the expression values are determined in the context of the methods of the present invention, an individualized “gene expression profile” is obtained that reflects the gene expression values of the GOI's in a sample or in subject. In the latter case, the expression values are also referred to as “subject expression profile”. It is noted that the sample expression profile and/or the subject expression profile may change as a function of time. For example, the gene expression profiles before or after therapeutic treatment of a patient with diagnosed prostate cancer may be different. Similarly, the genes expression profiles found in a sample containing prostate cancer cells will be different from those found in a sample that does not contain prostate cancer cells.

The term “marker gene(s)” or “gene(s) of interest” as used herein, thus relates to a set of gene(s), genetic unit(s) or sequence(s) (nucleotide sequence(s)) as defined herein above, whose expression level is either up-regulated or down-regulated in a prostate cancer cell or tissue or in any type of sample comprising such cells or tissues or portions or fragments thereof, when comparing to a control level, preferably when comparing to the expression in prostate tissue obtained at a different point in time, e.g. at initial diagnosis, directly after primary treatment, or later, i.e. several months or years after initial diagnosis or primary treatment. The term refers to any expression product of said genetic unit or sequence, in particular to an mRNA transcript. When the marker genes or genes of interest are examined for the first time, i.e. when a patient is for the first time suspected to have a prostate cancer, it is also contemplated to use additional control material that is derived from normal tissue or benign prostate tumor tissue.

The term “expression level” as used herein refers to the amount of marker gene transcript(s) derivable from a defined number of cells or a defined tissue portion, preferably to the amount of transcript obtainable in a standard nucleic acid (e.g. RNA) extraction procedure. Suitable extraction methods are known to the person skilled in the art.

The term “control level” (or “control state”), as used herein, relates to an expression level which may be determined at the same time and/or under similar or comparable conditions as the test sample by using (a) sample(s) previously collected and stored from a patient/patients whose condition or disease state, e.g. non-cancerous, normal or benign prostate tumor, advanced prostate cancer etc. is/are known. For the determination of a predisposition for aggressive cancer versus indolent cancer, the control level is preferably determined in cancer tissue obtained to establish the initial diagnosis, which is then compared to the expression of GOI's that is determined at a later stage, e.g. after primary treatment. The term “disease state” or “cancerous disease state” relates to any state or type of cellular or molecular condition between a non-cancerous cell state and (including) a terminal cancerous cell state. Preferably, the term includes different cancerous proliferation/developmental stages or levels of tumor development in the organism between (and excluding) a non-cancerous cell state and (including) a terminal cancerous cell state. Such developmental stages may include all stages of the TNM (Tumor, Node, Metastasis) classification system of malignant tumors as defined by the UICC, e.g. stages 0 and I to IV. The term also includes stages before TNM stage 0, e.g. developmental stages in which cancer biomarkers known to the person skilled in the art show a modified expression or expression pattern.

The expression level as mentioned above may preferably be the expression level of marker gene(s) or GOI's as defined herein.

The term “cancerous” relates in the context of the present invention to a cancerous disease state as defined herein above.

The term “non-cancerous” relates in the context of the present invention to a condition in which neither benign nor malign proliferation can be detected. Suitable means for said detection are known in the art.

The expression control level may be determined by a statistical method based on the results obtained by analyzing previously determined expression level(s) of the marker gene(s) of the present invention in samples from subjects whose disease state is known. Furthermore, the control level can be derived from a database of expression patterns or expression levels from previously tested patients, tissues or cells. The control level can be determined from a reference sample derived from a patient who has been diagnosed to suffer from prostate cancer, e.g. from hormone-independent or hormone-resistant prostate cancer. Moreover, the expression level of the marker genes of the present invention in a biological sample to be tested may be compared to multiple control levels, whose control levels are determined from multiple reference samples. It is contemplated to use a control level determined from a reference sample derived from a tissue type similar to that of the subject-derived biological sample. It is particularly preferred to use sample(s) derived from a patient whose disease state is cancerous as defined herein above.

In general diagnostic methods of prostate cancer using the herein described marker genes, it also possible to determine the control level in tissues of subjects that present a health condition in which neither benign nor malign proliferation can be detected. It is also contemplated to use sample(s) derived from a patient/patients having benign prostate tumor as defined herein above, i.e. which present a health condition in which benign proliferation can be detected.

The term “benign prostate tumor” as used herein refers to a prostate tumor which lacks all three of the malignant properties of a cancer, i.e. does not grow in an unlimited, aggressive manner, does not invade surrounding tissues, and does not metastasize. Typically, a benign prostate tumor implies a mild and non-progressive prostate neoplastic or swelling disease lacking the invasive properties of a cancer. Furthermore, benign prostate tumors are typically encapsulated, and thus inhibited in their ability to behave in a malignant manner. A benign tumor or a healthy condition may be determined by any suitable, independent molecular, histological or physiological method known to the person skilled in the art.

Alternatively, reference samples may comprise material derived from cell lines, e.g. immortalized cancer cell lines. Preferably, material derived from prostate cancer cell lines, may be comprised in a reference sample according to the present invention. Examples of preferred cancer cell lines comprise cells lines PC346P, PC346B, LNCaP, VCaP, DuCaP, PC346C, PC3, DU145, PC346CDD, PC346Flu1, PC346F1u2.

In a further, preferred alternative, reference samples may be derived from subject/patient tissues, or tissue panels or tissue collections obtained in clinical environments. The samples may, for example, be obtained from male patients undergoing surgery. The samples may be derived from any suitable tissue type, e.g. from prostate tissue or lymph nodes. Preferred examples of patient tissue collections are derived from surgical procedures (e.g., prostatectomy).

Moreover, it is contemplated to use the standard value of the expression levels of the marker gene(s) of the present invention in a population with a known disease state, e.g. a population having benign prostate tumor or a healthy population. The standard value may be obtained by any method known in the art. For example, a range of mean ±2 SD (standard deviation) or mean ±3 SD may be used as standard value.

Furthermore, the control level may also be determined at the same time and/or under similar or comparable conditions as the test sample by using (a) sample(s) previously collected and stored from a patient/patients whose disease state is/are known to be cancerous, i.e. who have independently been diagnosed to suffer from prostate cancer, in particular from aggressive prostate cancer.

In the context of the present invention, a control level determined from a biological sample that is known not to be cancerous, e.g. is a healthy tissue sample or a benign prostate tumor sample, is called “normal control level”.

If the control level is determined from a cancerous biological sample, in particular a sample from a patient for which prostate cancer was diagnosed independently, it may be designated as “cancerous control level”.

The term “prostate cancer” relates to a cancer of the prostate gland in the male reproductive system, which occurs when cells of the prostate mutate and begin to multiply out of control. Typically, prostate cancer is linked to an elevated level of prostate-specific antigen (PSA). In one embodiment of the present invention the term “prostate cancer” relates to a cancer showing PSA levels above 4.0. In another embodiment the term relates to cancer showing PSA levels above 2.0. The term “PSA level” refers to the concentration of PSA in the blood in ng/ml.

The term “prostate cancer” also comprises prostate cancer or prostate cancer cell lines that are sensitive on male sex hormone stimulation as far as their growth or proliferation is concerned. The term “sensitive” relates to situations in which the prostate cancer or prostate cancer cell line shows a biochemical or cellular reaction pattern in the presence of male sex hormones, but does need a male sex hormone for growth and/or proliferation. A hormone-sensitive prostate is accordingly understood as a malignant prostate tumor that has developed from pre-malignant forms like PIN (Prostate Intraepithelial Neoplasia) and is characterized by the fact that its growth is still dependent on the presence of the male sex hormones androgens. In contrast, once such tumors are treated by hormone depletion therapies those cancers typically develop into a hormone-resistant form that grows independent of the presence of androgens.

The term “hormone-resistant prostate cancer” means that the growth and proliferation of prostate cancer or prostate cancer cell lines is resistant to male sex hormone stimulation. The term also relates to a late prostate cancer developmental stage which is no longer amenable to an administration of anti-hormones, preferably anti-androgens as defined herein above.

The term “male sex hormone” as used herein refers to an androgen, preferably to testosterone, androstenedione, dihydrotestosterone, dehydroepiandrosterone, androstenediol or androsterone.

In a further aspect the present invention relates to the use of marker gene(s) or GOI's as a marker for diagnosing, detecting, monitoring or prognosticating malignant, hormone-sensitive prostate cancer or the progression towards prostate cancer.

The term “up-regulated” or “up-regulated expression level” or “increase of expression level” (which may be used synonymously) in the context of the present invention thus denotes a raise in the expression level between a situation to be analyzed, e.g. a situation derivable from a patient's sample, and a reference point, which could either be a normal control level or cancerous control level derivable from any suitable prostate tumor or cancer stage known to the person skilled in the art. Expression levels are deemed to be “up-regulated” when the gene expression, e.g. in a sample to be analyzed, differs by, i.e. is elevated by, for example, 5%, 6%, 7%, 8%, 9%, 10%, 15%, 20%, 25%, 30%, 40%, 50%, or more than 50% in comparison to a control level, or by at least 0.1 fold, at least 0.2 fold, at least 1 fold, at least 2 fold, at least 5 fold, or at least 10 fold or more in comparison to a control level. The control level may either be a normal control level or a cancerous control level as defined herein above. If a comparison with a cancerous control level is to be carried out, an additional comparison with a normal control level is preferred. Such an additional comparison allows for the determination of a tendency of the modification, e.g. the magnitude of an increase of the expression level may be observed and/or corresponding conclusions may be drawn. Preferred is a comparison to a benign prostate tumor, or to a healthy tissue or a sample derived from a healthy individual.

The term “down-regulated” or “down-regulated expression level” or “decrease of expression level” (which may be used synonymously) in the context of the present invention thus denotes a drop in the expression level between a situation to be analyzed, e.g. a situation derivable from a patient's sample, and a reference point, which could either be a normal control level or preferably cancerous control level derivable from any suitable prostate tumor or cancer stage known to the person skilled in the art. Expression levels are deemed to be “down-regulated” when the gene expression, e.g. in a sample to be analyzed, differs by, i.e. is decreased by, for example, 5%, 6%, 7%, 8%, 9%, 10%, 15%, 20%, 25%, 30%, 40%, 50%, or more than 50% in comparison to a control level, or by at least 0.1 fold, at least 0.2 fold, at least 1 fold, at least 2 fold, at least 5 fold, or at least 10 fold or more in comparison to a control level. The control level may either be a normal control level or a cancerous control level as defined herein above. If a comparison with a cancerous control level is to be carried out, an additional comparison with a normal control level is possible. Such an additional comparison allows for the determination of a tendency of the modification, e.g. the magnitude of decrease of the expression level may be observed and/or corresponding conclusions may be drawn. Preferred is a comparison to a benign prostate tumor, or to a healthy tissue or a sample derived from a healthy individual.

The expression values of the gene(s) of interest referred to herein can be used to establish a “Prostate Cancer Progression Index (PCPI)” using the following equation:

PCPI=(GEV_av_GOI_up)−(GEV_av_GOI_down),

wherein GOI refers to selected Genes of Interest, e.g. (bio-)marker genes, which are grouped into those down-regulated between patient groups with and without progression after primary treatment compared to those up-regulated between patient groups with and without progression after primary treatment. An average gene expression value for the down-regulated genes is calculated (GEV_av_GOI_down) as well as an average gene expression value for the up-regulated genes (GEV_av_GOI_up).

A specific embodiment a subject sample specific prostate cancer progression index is calculated according to the following equation:

PCPI=(GEV_av_GOI_up)−(GEV_av_GOI_down),

wherein GEV_av_GOI_up is an average gene expression value of up-regulated genes, and wherein GEV_av_GOI_down is an average gene expression value of a down-regulated genes, wherein the values are determined on the basis of normalized gene expression data per each patient sample and wherein “up-regulated genes” refer to genes up-regulated between patient groups with and without progression after primary treatment and “down-regulated genes” refer to genes down-regulated between patient groups with and without progression after primary treatment.

A selection of genes potentially relevant to patient stratification in prostate cancer or to determine the predisposition to develop aggressive prostate cancer after primary treatment is provided e.g. in FIG. 2. FIG. 2 provides an overview of 57 selected genes that were found to be significantly (p<0.05) differentially expressed and were regulated between patient groups in the same direction (i.e., either up- or down-regulated). These 57 genes may be used as GOI's according to one aspect of the invention. It is also possible to use selected genes from the above 57 genes, e.g. at least 17 genes, at least 18 genes, at least 19 genes, at least 20 genes, at least 21 genes, at least 22 genes, at least 23 genes, at least 24 genes, at least 25 genes, at least 26 genes, at least 27 genes, at least 28 genes, at least 29 genes, at least 30 genes, at least 31 genes, at least 32 genes, at least 33 genes, at least 34 genes, at least 35 genes, at least 36 genes, at least 37 genes, at least 38 genes, at least 39 genes, at least 40 genes, at least 41 genes, at least 42 genes, at least 43 genes, at least 44 genes, at least 45 genes, at least 46 genes, at least 47 genes, at least 48 genes, at least 49 genes, at least 50 genes, or more.

In another aspect a gene expression prognostic signature comprised of a combination of individual genes is provided, wherein the expression level of these GOI's can be measured using, e.g. RNA extracted from human prostate cancer tissue samples in a quantitative manner. The combination of these features into a single data model (i.e. the PCPI) provides significantly improved classification power to predict the progression of prostate cancer.

In one preferred embodiment the PCPI signature comprises the following 19 genes: PDE4D5, PDE4D7, AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2.

The term “phosphodiesterase 4D5” or “PDE4D5” relates to the splice variant 5 of the human phosphodiesterase PDE4D, i.e. the human phosphodiesterase PDE4D5 gene, preferably to the sequence as defined in NCBI Reference Sequence: NM_001197218.1, more preferably to the nucleotide sequence as set forth in SEQ ID NO: 1, which corresponds to the sequence of the above indicated NCBI Reference Sequence of the PDE4D5 transcript, and also relates to the corresponding amino acid sequence as set forth in SEQ ID NO: 2, which corresponds to the protein sequence defined in NCBI Protein Accession Reference Sequence NP_001184147.1 encoding the PDE4D5 polypeptide. The term “phosphodiesterase 4D5” or “PDE4D5” also relates to the amplicon that can be generated by the primer pair PDE1D5_forward (SEQ ID NO: 3) and the PDE1D5_reverse (SEQ ID NO: 4) and can be detected by probe SEQ ID NO: 5.

The term “phosphodiesterase 4D7” or “PDE4D7” relates to the splice variant 7 of the human phosphodiesterase PDE4D, i.e. the human phosphodiesterase PDE4D7 gene, preferably to the sequence as defined in NCBI Reference Sequence: NM_001165899.1, more preferably to the nucleotide sequence as set forth in SEQ ID NO: 6, which corresponds to the sequence of the above indicated NCBI Reference Sequence of the PDE4D7 transcript, and also relates to the corresponding amino acid sequence as set forth in SEQ ID NO: 7, which corresponds to the protein sequence defined in NCBI Protein Accession Reference Sequence NP_001159371.1 encoding the PDE4D7 polypeptide. The term “phosphodiesterase 4D7” or “PDE4D7” also relates to the amplicon that can be generated by the primer pair PDE1D7_forward (SEQ ID NO: 8) and the PDE1D7_reverse (SEQ ID NO: 9) and can be detected by probe SEQ ID NO: 10.

The term “AZGP1” relates to the Alpha-2-Glycoprotein 1-Zinc-Binding gene, preferably to the sequence as defined in NCBI Reference Sequence: NM_001185.3, more preferably to the nucleotide sequence as set forth in SEQ ID NO: 11, which corresponds to the sequence of the above indicated NCBI Reference Sequence of the AZGP1 transcript, and also relates to the corresponding amino acid sequence as set forth in SEQ ID NO: 12, which corresponds to the protein sequence defined in NCBI Protein Accession Reference Sequence NP_001176.1 encoding the AZGP1 polypeptide. The term “AZGP1” also relates to the amplicon that can be generated by the primer pair AZGP1_forward (SEQ ID NO: 13) and the AZGP1_reverse (SEQ ID NO: 14) and can be detected by probe SEQ ID NO: 15.

The term “FBLN1” relates to the Fibulin1 gene, preferably to one or more of the sequences as defined in NCBI Reference Sequence: NM_001996.3, NM_006485.3, NM_006486.2 and NM_006487.2, more preferably to the nucleotide sequences as set forth in SEQ ID NO: 16, 17, 18 and 19, respectively, which correspond to the sequences of the above indicated NCBI Reference Sequences of the Fibulin1 transcripts, and also relate to the corresponding amino acid sequence as set forth in SEQ ID NO: 20, 21, 22 and 23 which correspond to the protein sequences defined in NCBI Protein Accession Reference Sequences NP_001987.2, NP_006476.2, NP_006477.2 and NP_006478.2, respectively, encoding Fibulin1 polypeptides. The term “FBLN1” also relates to the amplicon(s) that can be generated by the primer pair FBLN1_forward (SEQ ID NO: 24) and the FBLN1_reverse (SEQ ID NO: 25) and can be detected by probe SEQ ID NO: 26.

The term “ILK” relates to the Integrin-Linked Kinase gene, preferably to one or more of the sequences as defined in NCBI Reference Sequences: NM_001014794.2, NM_001014795.2, NM_001278441.1, NM_001278442.1 and NM_004517.3, more preferably to the nucleotide sequences as set forth in SEQ ID NO: 27, 28, 29, 30 and 31, respectively, which correspond to the sequences of the above indicated NCBI Reference Sequences of the Integrin-Linked Kinase transcripts, and also relate to the corresponding amino acid sequence as set forth in SEQ ID NO: 32, 33, 34, 35 and 36 which correspond to the protein sequences defined in NCBI Protein Accession Reference Sequences NP_001014794.1, NP_001014795.1, NP_001265370.1, NP_001265371.1 and NP_004508.1 respectively, encoding Integrin-Linked Kinase polypeptides. The term “ILK” also relates to the amplicon(s) that can be generated by the primer pair ILK_forward (SEQ ID NO: 37) and the ILK_reverse (SEQ ID NO: 38) and can be detected by probe SEQ ID NO: 39.

The term “KRT15” relates to the Keratin 15 gene, preferably to the sequence as defined in NCBI Reference Sequence: NM_002275.3, more preferably to the nucleotide sequence as set forth in SEQ ID NO: 40, which corresponds to the sequence of the above indicated NCBI Reference Sequence of the KRT15 transcript, and also relates to the corresponding amino acid sequence as set forth in SEQ ID NO: 41, which corresponds to the protein sequence defined in NCBI Protein Accession Reference Sequence NP_002266.2 encoding the KRT15 polypeptide. The term “KRT15” also relates to the amplicon that can be generated by the primer pair KRT15_forward (SEQ ID NO: 42) and the KRT15_reverse (SEQ ID NO: 43) and can be detected by probe SEQ ID NO: 44.

The term “MEIS2” relates to the Meis Homeobox 2 preferably to one or more of the sequences as defined in NCBI Reference Sequences: NM_001220482.1, NM_002399.3, NM_170674.4, NM_170675.4, NM_170676.4, NM_170677.4, NM_172315.2 and NM_020149.2 more preferably to the nucleotide sequences as set forth in SEQ ID NO: 45, 46, 47, 48, 49, 50, and/or 51, respectively, which correspond to the sequences of the above indicated NCBI Reference Sequences of the MEIS2 transcripts, and also relate to the corresponding amino acid sequence as set forth in SEQ ID NO: 53, 54, 55, 56, 57, 58, 59 and/or 60 which correspond to the protein sequences defined in NCBI Protein Accession Reference Sequences NP_001207411.1, NP_002390.1, NP_733774.1, NP_733775.1, NP_733776.1, NP_733777.1, NP_758526.1 and NP_758527.1 respectively, encoding MEIS2 polypeptides. The term “MEIS2” also relates to the amplicon(s) that can be generated by the primer pair MEIS2_forward (SEQ ID NO: 61) and the MEIS2_reverse (SEQ ID NO: 62) and can be detected by probe SEQ ID NO: 63.

The term “MYBPC1” relates to the Myosin Binding Protein C, Slow Type preferably to one or more of the sequences as defined in NCBI Reference Sequences: NM_001254718.1, NM_001254719.1, NM_001254720.1, NM_001254721.1, NM_001254722.1, NM_001254723.1, NM_002465.3, NM_206819.2, NM_206820.2 and NM_206821.2 more preferably to the nucleotide sequences as set forth in SEQ ID NO: 64, 65, 66,67, 68, 69, 70, 71, 72 and/or 73, respectively, which correspond to the sequences of the above indicated NCBI Reference Sequences of the MYBPC1 transcripts, and also relate to the corresponding amino acid sequence as set forth in SEQ ID NO: 74, 75, 76, 77, 78, 79, 80, 81, 82 and/or 83 which correspond to the protein sequences defined in NCBI Protein Accession Reference Sequences NP_001241647.1, NP_001241648.1, NP_001241649.1, NP_001241650.1, NP_001241651.1, NP_001241652.1, NP_002456.2 ,NP_996555.1, NP_996556.1 and NP_996557.1 respectively, encoding MYBPC1 polypeptides. The term “MYBPC1” also relates to the amplicon(s) that can be generated by the primer pair MYBPC1_forward (SEQ ID NO: 84) and the MYBPC1_reverse (SEQ ID NO: 85) and can be detected by probe SEQ ID NO: 86.

The term “PAGE4” relates to the P Antigen Family, Member 4 gene, preferably to the sequence as defined in NCBI Reference Sequence: NM_007003.3, more preferably to the nucleotide sequence as set forth in SEQ ID NO: 87, which corresponds to the sequence of the above indicated NCBI Reference Sequence of the PAGE4 transcript, and also relates to the corresponding amino acid sequence as set forth in SEQ ID NO: 88, which corresponds to the protein sequence defined in NCBI Protein Accession Reference Sequence NP_008934.1 encoding the PAGE4 polypeptide. The term “PAGE4” also relates to the amplicon that can be generated by the primer pair PAGE4_forward (SEQ ID NO: 89) and the PAGE4_reverse (SEQ ID NO: 90) and can be detected by probe SEQ ID NO: 91.

The term “SRD5A2” relates to the Steroid 5-Alpha-Reductase 2 gene, preferably to the sequence as defined in NCBI Reference Sequence: NM_000348.3, more preferably to the nucleotide sequence as set forth in SEQ ID NO: 92, which corresponds to the sequence of the above indicated NCBI Reference Sequence of the SRD5A2 transcript, and also relates to the corresponding amino acid sequence as set forth in SEQ ID NO: 93, which corresponds to the protein sequence defined in NCBI Protein Accession Reference Sequence NP_000339.2 encoding the SRD5A2 polypeptide. The term “SRD5A2” also relates to the amplicon that can be generated by the primer pair SRD5A2_forward (SEQ ID NO: 94) and the SRD5A2_reverse (SEQ ID NO: 95) and can be detected by probe SEQ ID NO: 96.

The term “COL1A1” relates to the Collagen, Type I, Alpha 1 gene, preferably to the sequence as defined in NCBI Reference Sequence: NM_000088.3, more preferably to the nucleotide sequence as set forth in SEQ ID NO: 97, which corresponds to the sequence of the above indicated NCBI Reference Sequence of the COL1A1 transcript, and also relates to the corresponding amino acid sequence as set forth in SEQ ID NO: 98, which corresponds to the protein sequence defined in NCBI Protein Accession Reference Sequence NP_000079.2 encoding the COL1A1 polypeptide. The term “COL1A1” also relates to the amplicon that can be generated by the primer pair COL1A1_forward (SEQ ID NO: 99) and the COL1A1_reverse (SEQ ID NO: 100) and can be detected by probe SEQ ID NO: 101.

The term “COL3A1” relates to the Collagen, Type III, Alpha 1gene, preferably to the sequence as defined in NCBI Reference Sequence: NM_000090.3, more preferably to the nucleotide sequence as set forth in SEQ ID NO: 102, which corresponds to the sequence of the above indicated NCBI Reference Sequence of the COL3A1 transcript, and also relates to the corresponding amino acid sequence as set forth in SEQ ID NO: 103, which corresponds to the protein sequence defined in NCBI Protein Accession Reference Sequence NP_000081.1 encoding the COL3A1 polypeptide. The term “COL3A1” also relates to the amplicon that can be generated by the primer pair COL3A1_forward (SEQ ID NO: 104) and the COL3A1_reverse (SEQ ID NO: 105) and can be detected by probe SEQ ID NO: 106.

The term “COL5A2” relates to the Collagen, Type V, Alpha 2 gene, preferably to the sequence as defined in NCBI Reference Sequence: NM_000393.3, more preferably to the nucleotide sequence as set forth in SEQ ID NO: 107, which corresponds to the sequence of the above indicated NCBI Reference Sequence of the COL5A2 transcript, and also relates to the corresponding amino acid sequence as set forth in SEQ ID NO: 108, which corresponds to the protein sequence defined in NCBI Protein Accession Reference Sequence NP_000384.2 encoding the SRD5A2 polypeptide. The term “COL5A2” also relates to the amplicon that can be generated by the primer pair COL5A2_forward (SEQ ID NO: 109) and the COL5A2_reverse (SEQ ID NO: 110) and can be detected by probe SEQ ID NO: 111.

The term “INHBA” relates to the Inhibin, Beta A gene, preferably to the sequence as defined in NCBI Reference Sequence: NM_002192.2, more preferably to the nucleotide sequence as set forth in SEQ ID NO: 112, which corresponds to the sequence of the above indicated NCBI Reference Sequence of the INHBA transcript, and also relates to the corresponding amino acid sequence as set forth in SEQ ID NO: 113, which corresponds to the protein sequence defined in NCBI Protein Accession Reference Sequence NP_002183.1 encoding the INHBA polypeptide. The term “INHBA” also relates to the amplicon that can be generated by the primer pair INHBA_forward (SEQ ID NO: 114) and the INHBA_reverse (SEQ ID NO: 115) and can be detected by probe SEQ ID NO: 116.

The term “THBS2” relates to the Thrombospondin 2 gene, preferably to the sequence as defined in NCBI Reference Sequence: NM_003247.3 more preferably to the nucleotide sequence as set forth in SEQ ID NO: 117, which corresponds to the sequence of the above indicated NCBI Reference Sequence of the THBS2 transcript, and also relates to the corresponding amino acid sequence as set forth in SEQ ID NO: 118, which corresponds to the protein sequence defined in NCBI Protein Accession Reference Sequence NP_003238.2 encoding the THBS2 polypeptide. The term “THBS2” also relates to the amplicon that can be generated by the primer pair THBS2_forward (SEQ ID NO: 119) and the THBS2_reverse (SEQ ID NO: 120) and can be detected by probe SEQ ID NO: 121.

The term “VCAN” relates to the Versican gene, preferably to one or more of the sequences as defined in NCBI Reference Sequences: NM_001126336.2, NM_001164097.1, NM_001164098.1 and NM_004385.4 more preferably to one or more nucleotide sequences as set forth in SEQ ID NO: 122, 123, 124 and 125, respectively, which correspond to the sequences of the above indicated NCBI Reference Sequences of the VCAN transcripts, and also relates to the corresponding amino acid sequences as set forth in SEQ ID NO: 126, 127, 128 and 129 which correspond to the protein sequences defined in NCBI Protein Accession Reference Sequences NP_001119808.1, NP_001157569.1, NP_001157570.1 and NP_004376.2 respectively, encoding VCAN polypeptides. The term “VCAN” also relates to the amplicon(s) that can be generated by the primer pair VCAN_forward (SEQ ID NO: 130) and the VCAN_reverse (SEQ ID NO: 131) and can be detected by probe SEQ ID NO: 132.

The term “BNG” relates to the Biglycan gene, preferably to the sequence as defined in NCBI Reference Sequence: NM_001711.4, more preferably to the nucleotide sequence as set forth in SEQ ID NO: 133, which corresponds to the sequence of the above indicated NCBI Reference Sequence of the BNG transcript, and also relates to the corresponding amino acid sequence as set forth in SEQ ID NO: 134, which corresponds to the protein sequence defined in NCBI Protein Accession Reference Sequence NP_001702.1 encoding the BNG polypeptide. The term “BNG” also relates to the amplicon that can be generated by the primer pair BNG_forward (SEQ ID NO: 135) and the BNG_reverse (SEQ ID NO: 136) and can be detected by probe SEQ ID NO: 137.

The term “BIRC5” relates to the Versican gene, preferably to one or more of the sequences as defined in NCBI Reference Sequences: NM_001012270.1, NM_001012271.1 and NM_001168.2 more preferably to one or more nucleotide sequences as set forth in SEQ ID NO: 138, 139 and 140, respectively, which correspond to the sequences of the above indicated NCBI Reference Sequences of the BIRC5transcripts, and also relates to the corresponding amino acid sequences as set forth in SEQ ID NO: 141, 142, and 143 which correspond to the protein sequences defined in NCBI Protein Accession Reference Sequences NP_001012270.1, NP_001012271.1 and NP_001159.2 respectively, encoding BIRC5polypeptides. The term “BIRC5” also relates to the amplicon(s) that can be generated by the primer pair BIRC5_forward (SEQ ID NO: 144) and the BIRC5_reverse (SEQ ID NO: 145) and can be detected by probe SEQ ID NO: 146.

The term “DYRK2” relates to the Versican gene, preferably to one or more of the sequences as defined in NCBI Reference Sequences: NM_003583.3 and NM_006482.2 more preferably to one or more nucleotide sequences as set forth in SEQ ID NO: 147 and 148, respectively, which correspond to the sequences of the above indicated NCBI Reference Sequences of the DYRK2 transcripts, and also relates to the corresponding amino acid sequences as set forth in SEQ ID NO: 149 and 150 which correspond to the protein sequences defined in NCBI Protein Accession Reference Sequences NP_003574.1 and NP_006473.2 respectively, encoding DYRK2 polypeptides. The term “DYRK2” also relates to the amplicon(s) that can be generated by the primer pair DYRK2_forward (SEQ ID NO: 151) and the DYRK2_reverse (SEQ ID NO: 152) and can be detected by probe SEQ ID NO: 153.

In other embodiments the PCPI signature can be an 18-gene list comprising PDE4D instead of PDE4D5 and PDE4D7, or it may comprise the remaining 17 genes. Therefore, in a preferred embodiment, the PCPI signature can be a 17-gene list which comprises the above-mentioned 17 genes and comprises neither PDE4D5 nor PDE4D7.

In another embodiment, the present invention provides a gene expression prognostic signature comprised of a combination of 19 individual genes which can be measured on extracted RNA from human prostate cancer tissue samples in a quantitative manner. The combination of these features into a single data model (=PCPI->Prostate Cancer Progression Index) provides significantly improved classification power to predict the progression of prostate cancer. In embodiments using either these 19 genes, or 17, 18 or 57, the PCPI referred to in the present invention is used in methods of diagnosing prostate cancer in a sample, prognosticating the patient's individual disease status and expected development of disease, and it provides a parameter in methods for stratifying and/or identifying patients for an individually preferred treatments or therapy, if applicable. Based on the PCPI parameter, the predisposition or likelihood that, e.g., the tumor progresses to aggressive prostate cancer can be determined. For example, if the PCPI obtained in the analysis of a tumor is higher than a defined cut-off (or threshold) level, the probability that said tumor progresses to an aggressive form is also higher, and vice versa.

The classification step of tumor samples that are analysed according to the methods described herein thus comprises the comparison of PCPI values between samples that have been obtained at a specific time point in the past (e.g. before initial diagnosis of prostate cancer) and samples obtained at a later stage. When the PCPI obtained in the analysis of a tumor sample at said later stage is higher than a defined threshold or cut-off, this parameter can be used as an indication of a progression towards aggressive prostate cancer.

The cut-off may be defined based on a reference data set comprising patient groups with or without progression. The cut-off may be set for example at 70%, 80%, 85%, 90%, 93%, 95%, 96%, 97%, 98% or 99% sensitivity with regard to classification of patients without or with progression. In a preferred embodiment the cut-off may be set at 90% sensitivity.

Of course, the PCPI may also be used in methods of establishing a patients' prognosis, in a method for the stratification of the patients into groups for an appropriate treatment, with the aid of which the physician comes to the conclusion that a specific treatment is not appropriate, that a therapy is not (yet) required, or that therapies are no longer needed in view of the patients overall physical condition.

Embodiments of the present invention thus relate to the identification and use of marker gene (or sequence) expression patterns (or “profiles” or “signatures”) which are clinically relevant to prostate cancer. The marker gene expression profiles, whether embodied in nucleic acid expression, protein expression, or other expression formats, may be used to predict prostate cancer recurrence and/or survival of subjects afflicted with prostate cancer. Particularly, the marker genes are used in establishing the herein described Prostate Cancer Progression Profile (PCPI) that is be used in the identification/classification of patients with an aggressive or indolent disease.

In the context of the present application, the expression “initial diagnosis” refers to the first or a subsequent diagnosis preceding the date on which one of the methods according to the present invention was performed, and the PCPI was calculated. For example the initial diagnosis may be established for a patient whose prostate tissue material was analyzed for the first time, and preferably before diagnosis of prostate cancer.

In the context of the present application, the term “predetermined period after initial diagnosis” comprises a period of about 6 months, 1 year, 2 years, 3 years, 4 years, 5 years, 6 years, 7 years, 8 years, 9 years, 10 years, 15 years or 20 years or more. In the context of the present application, the expression “patient with newly diagnosed prostate cancer” refers to an individual who has been diagnosed as having biochemical values, clinical values or PCPI values that confirm or raise suspicion of the presence of prostate cancer cells in a sample taken from this patient for the first time.

In the context of the present application, the expression “primary treatment for prostate cancer” refers to any known method for the treatment of prostate cancer, i.e. surgery, chemotherapy, treatment with biologicals, radiation therapy, and the like.

In the context of the present application, the expression “treatment with biologicals” refers to the treatment of prostate cancers with monoclonal antibodies or derivatives thereof, or peptides or fragments thereof that specifically inhibit or block or neutralize biological molecules in vivo as well as in vitro that are implicated in growth, proliferation, maintenance, or in any other metabolic pathway that is used by prostate cancer cells. Monoclonal antibodies directed to established targets include those that are approved for solid tumors such as anti-human EGFR-2 monoclonal antibodies such as trastuzumab, anti-EGFR antibodies such as cituximab and panitumumab and the anti-vascular endothelial growth factor (VEGF) monoclonal antibody bevacizumab.

In the context of the present application, the expression “biochemical recurrence” refers, e.g., to recurrent biological values of increased PSA indicating the presence of prostate cancer cells in a sample. However, it is also possible to use other markers that can be used in the detection of the presence or that rise suspicion of such presence.

In the context of the present application, the term “clinical recurrence” refers to the presence of clinical signs or clinical values as measured, for example using the Gleason Score.

In the context of the present application, the expression “predisposition of prostate cancer that is susceptible to disease progression” refers to a measurable and significant parameter (for example the PCPI), which provides an indication that the likelihood that the patient from whom a sample was investigated will develop aggressive prostate cancer, i.e. after primary treatment of diagnosed prostate cancer, for example by prostatectomy, chemotherapy and/or radiation therapy, prostate cancer cells re-emerged which have a potential for aggressive growth, such as the formation of metastases in the lymph nodes and bones.

In the context of the present application, the expression “computer implemented method for determining a prognosis of a patient having a prostate cancer” refers to a method wherein software algorithms calculate a PCPI and based thereon provide a prognosis for the patient that is analyzed, wherein this method uses raw data obtained upon measurement of the gene expression level of the genes referred to herein and conversion thereof into a PCPI using the above-described equation.

In the context of the present application, the methods for diagnosis, prognosis, identification of a predisposition for development or progression of aggressive prostate cancer, the identification of patients suffering from prostate cancer or subject to recurrence of prostate cancer after primary treatment, may be treated with a suitable therapeutic regime on the basis of the results obtained in any of the methods according to the present invention. Thus, for example, if the prognosis is that a patient will develop aggressive prostate cancer based on results determining a predisposition for this type of cancer, the physician may decide whether or not to use any of the available therapies for the treatment of prostate cancer, such as surgery, chemotherapy, radiation therapy, hormone therapy or combinations thereof and the like.

The methods according to the present invention also provide information with the aid of the PCPI based on which it can be decided whether or not a patient should be subjected to surgery, chemotherapy, radiation therapy, or whether a further biopsy is to be performed. Thus, the PCPI represents an important parameter in establishing an individualized diagnosis, prognosis, allows to identify patients with a predisposition for aggressive prostate cancer, or to stratify the same for the therapy that is best suited for this patient. The PCPI can also be used in combination with other means and methods to establish a diagnosis or prognosis, and to identify or stratify patients as discussed herein. The present invention thus also relates to prostate cancer-specific therapies or treatments as pointed out above for use in the treatment of patients that have been identified as eligible for such therapies on the basis of the PCPI, optionally in combination with other diagnostic or prognostic methods, with a view to selecting the most suitable cancer treatment for said patient. For example, the complete removal of lymph nodes may be an option in the treatment of patients that have been identified or diagnosed or for whom a prognosis has been established using the methods according to the invention, when the PCPI is high compared with a control sample.

In embodiments of the present invention involving patients, who were treated by prostatectomy after initial clinical diagnosis, the invention includes methods to predict the likelihood or predisposition of prostate cancer recurrence, cancer metastasis, and/or recurrence of elevated PSA levels.

Further embodiments of the present invention relate to methods to identify, and then use, marker gene (or sequence) expression profiles in a sample which provide prognostic information related to prostate cancer. The expression profiles correlate with (and so are able to discriminate between) patients with good or poor cancer recurrence, cancer metastasis, and/or survival outcomes.

In embodiments of the present invention a method to identify marker gene expression profiles relevant to cancer recurrence and/or metastasis in prostate cancer afflicted patients is provided.

In further embodiments of the invention, methods comparing marker gene (or sequence) expression in a sample comprising gene expression analysis in prostate cancer cells from a patient to an identified expression profile, the PCPI, are provided to determine the likely outcome for the patient, for example after prostatectomy, chemotherapy, radiation therapy, etc. In further aspects of these embodiments of the invention, the obtained information may be advantageously used to predict whether a patient will likely benefit from, e.g., surgery to treat prostate cancer or whether a patient will be better off with another type of treatment or need adjuvant treatment in addition to surgery. Patients that will likely benefit from surgery, such as a radical prostatectomy, can be selected on the basis of the expression profile of the patient's prostate sample as disclosed herein, to be free of cancer recurrence and/or metastasis following the surgery. In a further embodiment, a patient that may be expected to not benefit from surgery is one predicted, by the expression profile of the patient's prostate cancer cells as disclosed herein, to develop cancer metastasis following the surgery. By way of example, the recurrence of prostate cancer may be at the same location while metastasis is frequently to a different location or tissue, such as to a lymph node or bone. Further embodiments of the present invention relate to a method to identify a patient, from a population of patients with prostate cancer cells, or following prostatectomy, as belonging to a subpopulation of patients with either a better cancer recurrence and/or survival outcome (such as lower risk of recurrence or metastasis), relative to another subpopulation, or as belonging to a subpopulation with a poorer cancer recurrence and/or survival outcome (such as elevated risk of recurrence or metastasis), relative to another subpopulation. The methods of the invention are thus allowing to stratify patients into subpopulations or subtypes.

Methods of the present invention relate to embodiments for the identification of patients with prostate cancer, optionally following prostatectomy, as likely to have a better or poorer cancer recurrence, cancer metastasis, and/or survival outcome by assaying for the PCPI disclosed herein. Where previously subjective interpretation of sample obtained from patients (such as that based upon immunohistochemical staining and subjective analysis) may have been used to determine the predisposition for aggressive versus indolent disease and/or treatment of prostate cancer patients, or pre- or post-prostatectomy patients, or prostate cancer patients that have been treated by a different therapy, the present invention introduces an objective index reflecting expression patterns, which may be used alone or in combination with other (subjective and/or objective) criteria to provide a more accurate assessment of patient outcomes, including survival and the recurrence or metastasis of cancer. The methods based on the analysis of the marker gene expression patterns of the invention thus include a means to detect/diagnose prostate cancer, or pre- or post-prostatectomy prognosis, or, more generally, to determine the predisposition to either aggressive or indolent disease pre- or post-primary treatment.

The PCPI is based on the analysis of more than one marker gene, or expressed sequence, capable of discriminating between prostate cancer stages, or pre- or post-prostatectomy prostate cancer stages with significant accuracy. The expression level(s) of the marker gene(s), or expressed sequence(s) of interest are identified as correlated with outcomes such that the expression level(s) is/are relevant to a determination of the preferred treatment protocols of a patient. Thus, the invention includes embodiments relating to a method to determine the outcome of a subject afflicted with prostate cancer, optionally pre- or post-prostatectomy, by assaying a prostate sample suspected to contain a cancer cell from said patient for expression levels that are correlated with patient outcomes according to the herein disclosed methods. In another embodiment of the invention method to determine the risk of prostate cancer recurrence and/or metastasis in a subject patient are disclosed.

In methods of the invention, a sample suspected to comprise prostate cancer cells from a patient is analyzed for the expression levels of marker genes disclosed herein. The aggregated expression levels of the marker genes can be compared with the mean or median expression level(s) thereof in a population of prostate cancer cells. This assessment may then be used to determine the risk of prostate cancer recurrence and/or metastasis in the subject from which the sample was obtained when the PCPI is used. The expression levels of the disclosed genes (or expressed sequences) are correlated with a low risk of cancer recurrence and/or metastasis, or a high risk of cancer recurrence and/or metastasis.

Put differently, the individual marker genes (or expressed sequences) disclosed herein are expressed at higher, or lower, levels (in comparison to normalized expression values in a prostate cancer cell population) such that the deviation is correlated with an higher or lower risk or predisposition for the development of aggressive versus indolent cancer in patients previously diagnosed with prostate cancer, optionally patients that have already been treated specifically for prostate cancer, or de novo diagnosed patients.

In some embodiments, assaying for expression levels may be performed by use of nucleic acid probes selected that are specifically recognizing the marker genes as disclosed herein. These probes hybridize, under appropriate conditions, and detect expressed sequences as disclosed herein. In embodiments of the present invention, probes can be used to detect a region of sequence amplified from an expressed marker (gene) sequence.

The detection or determination of expression levels of marker (gene) sequences may be referred to as detecting or determining an expression profile or signature. Expression patterns of the invention comprising disclosed marker gene sequences are identified and used as described herein. For their identification, a large sampling of the gene expression levels in a sample containing prostate cancer cells is obtained through quantifying the expression levels of mRNA. In one embodiment, the invention includes detecting marker gene (or sequence) expression levels by analyzing gene expression from single cells or homogenous cell populations which have been dissected away from, or otherwise isolated or purified from, contaminating cells beyond that possible by a simple biopsy. Since the expression of numerous genes fluctuates between cells from different patients as well as between cells from the same patient sample, multiple data from expression of individual gene sequences are used as reference data to generate models which in turn permit the identification of individual genes and sequences, the expression of which are correlated with particular prostate cancer, or pre- or post-prostatectomy, outcomes, or which allow classifying patients according to their predisposition for aggressive versus indolent cancer. In these methods, the gene expression levels are preferably converted into a PCPI as defined above, using the marker genes of interest referred to in the claims and throughout the present the present application.

According to the present invention, marker gene expression levels of various genes in these models were then analyzed to identify nucleic acid sequences, the expressions of which are positively, or negatively, correlated, with a prostate cancer, or post-prostate cancer treatment, outcome.

Preferred embodiments of the present invention is based on the identification of a subset of expressed marker gene sequences in a cell, identified as correlating to outcomes as described herein. An expression pattern or profile of the invention includes a combination of these identified marker sequences (or genes) that are converted into a PCPI. The use of multiple samples for identification of expressed sequences increases the confidence with which a gene is considered to correlate with a prostate cancer recurrence, metastasis, and/or survival outcome. Without sufficient confidence, it remains unpredictable whether a particular gene is actually correlated with an outcome and also unpredictable whether expression of a gene may be successfully used to identify the outcome for a prostate cancer, or pre- or post-prostatectomy, subject or patient. The identified, nucleic acid sequences corresponding to the disclosed marker genes (or expressed sequences) of interest may be selected for assessment as an expression profile to be detected or assessed for its predictive properties.

A profile of expression levels or PCPI that is highly correlated with one outcome relative to another may be used to assay a prostate cancer cell containing sample from a subject or patient to predict the outcome of the subject from whom the sample was obtained. Such an assay may be used as part of a method to determine the therapeutic treatment for said subject based upon the outcome identified. This correlation provides a molecular determination of prostate cancer recurrence, cancer metastasis, and/or survival outcomes as disclosed herein. Without being bound by theory, and offered to improve understanding of the instant invention, the disclosed molecular determination is more powerful than other molecular indicators related to prostate cancer, such as the determination of prostate serum antigen (PSA) levels. Additional uses of the correlated expression patterns and profiles are in the classification of cells and tissues; determination of diagnosis and/or prognosis; and determination and/or alteration of therapy.

The ability to discriminate is conferred by the identification of expression of the individual marker gene sequences as relevant and not by the form of the assay used to determine the actual level of expression. The methods of the invention reflect, quantitatively or qualitatively, expression of marker gene sequence(s) in the “transcriptome” (the transcribed fraction of genes in a genome). The nature of the prostate cancer cell containing sample is not limiting, as fresh tissue, freshly frozen tissue, and fixed tissue, such as formalin-fixed paraffin-embedded (FFPE) tissues, may be used in the disclosed methods. In some embodiments, the sample may be that of a needle (core) biopsy or other biopsy.

For detecting an identified expression pattern or PCPI signature, embodiments of the invention include detecting gene (or sequence) expression patterns by gathering global, or near global, gene expression data from single cells or homogenous cell populations which have been dissected away from, or otherwise isolated or purified from, contaminating cells beyond that possible by a simple biopsy. The expression levels of the marker genes (sequences) in the pattern or profile are then detected or otherwise measured. In other embodiments, a method may only detect or measure the expression levels of the genes (sequences) in the profile without assessment or other determination of the expression of other genes or sequences.

In an additional aspect, the analysis of expression levels may be performed in combination with, or in place of, other assessments or indicators of prostate cancer. In some embodiments, the analysis is made in combination with a method of determining the grade of prostate cancer in a sample comprising prostate cancer cells from a subject. In other embodiments, the combination is with a method of determining the stage of prostate cancer in the sample. A third possibility is combination with detecting or determining PSA levels in the subject, optionally before a procedure used to isolate the prostate cancer cells. Of course a combination with any one, two, or all three of these representative examples is possible. Whenever more than one type of assessment is used, the result is a multivariate analysis. The invention expressly includes all possible combinations of assessments described herein as multivariate embodiments.

Generally, any accepted method of assessing prostate cancer grade and/or stage as known to the skilled person may be used. In some cases, the method of determining prostate cancer grade comprises determination of a Gleason Score (or Gleason Grade). In other cases, the method of determining prostate cancer stage comprises a determination according to the American Joint Committee on Cancer (AJCC) tumor staging system for assessing prostate cancer stage. And as described herein, the analysis of gene (sequence) expression levels may be performed in place of or in combination with either the Gleason Score or the AJCC tumor stage determination. In cases of PSA levels, its assessment may be conducted before a prostatectomy which is used to provide a sample comprising prostate cancer cells for use in any method described herein.

In further embodiments, the invention includes physical and methodological means for detecting the expression of genes (or sequences) disclosed herein. These means may be directed to assaying one or more aspect of the DNA template(s) underlying the expression of the gene (or sequence), of the RNA used as an intermediate to express the gene (or sequence), or of the proteinaceous product expressed by the gene (or sequence).

While the present invention is described mainly in the context of human prostate cancer, it may be practiced in the context of prostate cancer of any animal known to be potentially afflicted by prostate cancer. Non-limiting examples of animals for the application of the present invention are mammals, particularly those important to agricultural applications (such as, but not limited to, cattle, sheep, horses, and other “farm animals”), animal models of prostate cancer, and animals for human companionship (such as, but not limited to, dogs and cats).

A gene expression “pattern” or “profile” or “signature” and the corresponding calculated PCPI refers to the relative expression of genes of interest (expressed sequences) between two or more prostate cancer, or pre- or post-prostatectomy, cancer recurrence, metastasis, and/or survival outcomes which expression is correlated with being able to distinguish between the outcomes.

A “gene” or “expressed sequence” is a polynucleotide that encodes, and expresses in a detectable manner, a discrete product, whether RNA or proteinaceous in nature. It is appreciated that more than one polynucleotide may be capable of encoding a discrete product. The term includes alleles and polymorphisms of a gene or expressed sequence that encodes the same product, or a functionally associated (including gain, loss, or modulation of function) analog thereof, based upon chromosomal location and ability to recombine during normal mitosis.

The terms “correlate” or “correlation” or equivalents thereof refer to an association between expression of two or more genes and a physiologic state of a prostate cell to the exclusion of one or more other states as identified by use of the methods as described herein. A gene may be expressed at higher or lower levels and still be correlated with one or more prostate cancer, or pre- or post-prostatectomy, state or outcome.

As used herein, a “marker gene” or “marker gene sequence” the expression of which is analyzed in the context of the present invention, may also be described as a gene of interest (GOI).

In embodiments of the invention, the strength of expression of a given marker gene (or expressed sequence) is normalized (such as relative to expression of a reference gene that is expressed at relatively constant levels, or the expression of the GOI is measured across all available samples and the expression value is normalized) to provide a normalized value or score for the correlation analysis of the marker gene expression signature (e.g. the PCPI) and other parameter, e.g. clinical data. In many cases, the expression data is normalized, median-centered, and log-transformed as known to the skilled person prior to further use, such as in clustering and discriminant analysis. Where more than one expression level is used (as in the case of a gene expression profile or index, e.g. the PCPI), the values or scores may be summed and then analyzed as an aggregate value for assessing the correlation or conducting classification based on the correlation.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence. It is possible to further use any sequencing method known in the art to identify the sequences of GOI's.

The term “corresponding” may refer to, where appropriate, a nucleic acid molecule as sharing a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17). Methods for amplifying mRNA are generally known in the art, and include reverse transcription PCR (RT-PCR) and those described in U.S. patent application Ser. No. 10/062,857 (filed on Oct. 25, 2001), as well as U.S. Provisional Patent Applications 60/298,847 (filed Jun. 15, 2001) and 60/257,801 (filed Dec. 22, 2000), all of which are hereby incorporated by reference in their entireties as if fully set forth. Another method which may be used is quantitative PCR (or Q-PCR). Alternatively, RNA may be directly labeled as the corresponding cDNA by methods known in the art.

The term “Cq value” defines that PCR cycle in qRT-PCR (quantitative real-time PCR) where the measured fluorescence crosses for the first time the background fluorescence.

A “microarray” is a linear or two-dimensional array of discrete regions, each having a defined area, formed on the surface of a generally solid support such as, but not limited to, glass, plastic, or synthetic membrane. The density of the discrete regions on a microarray is determined by the total numbers of immobilized polynucleotides to be detected on the surface of a single solid phase support, such as at least about 50/cm2, at least about 100/cm2, at least about 500/cm2, but below about 1,000/cm2 in some embodiments. The arrays may contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Because the position of each particular group of polynucleotides in the array is known, the identities of a sample polynucleotides can be determined based on their binding to a particular position in the microarray.

Because the invention relies upon the identification of genes (or expressed sequences) that are over- or under-expressed, one embodiment of the invention involves determining expression by hybridization of mRNA, or an amplified or cloned version thereof (such as DNA or cDNA), of a sample cell to a polynucleotide that is unique to a particular gene sequence. Polynucleotides of this type may contain at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Other embodiments may use polynucleotides of at least or about 50, at least or about 100, at least about or 150, at least or about 200, at least or about 250, at least or about 300, at least or about 350, or at least or about 400 basepairs of a gene sequence that is not found in other gene sequences. The term “about” as used in the preceding sentence refers to an increase or decrease of 10% from the stated numerical value. Such polynucleotides may also be referred to as polynucleotide probes that are capable of hybridizing to sequences of the genes, or unique portions thereof, described herein. In many cases, the hybridization conditions are stringent conditions of about 30% v/v to about 50% formamide and from about 0.01M to about 0.15M salt for hybridization and from about 0.01M to about 0.15M salt for wash conditions at about 55 to about 65° C. or higher, or conditions equivalent thereto.

In other embodiments, polynucleotide probes for use in the invention may have about or 95%, about or 96%, about or 97%, about or 98%, or about or 99% identity with the marker gene sequences the expression of which shall be determined. Identity is determined using the BLAST algorithm, as described above. These probes may also be described on the basis of the ability to hybridize to expressed marker genes used in methods of the invention under stringent conditions as described above or conditions equivalent thereto.

In many cases, the sequences are those of mRNA encoded by the marker genes, the corresponding cDNA to such mRNAs, and/or amplified versions of such sequences. In some embodiments of the invention, the polynucleotide probes are immobilized on an array, other devices, or in individual spots that localize the probes.

Suitable labels that can be used according to the invention, include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means. The term “support” refers to conventional supports such as beads, particles, dipsticks, fibers, filters, membranes and silane or silicate supports such as glass slides.

As used herein, a “prostate tissue sample” or “prostate cancer cell sample” refers to a sample of prostate tissue isolated from an individual, such as one afflicted with prostate cancer. The sample may be from material removed via a prostatectomy, such as a radical prostatectomy. Alternatively, they are obtained by other means, such as needle (core) biopsy or other biopsy techniques, like laterally directed biopsies, the conventional sextant biopsy approach, different combinations of sextant and lateral biopsies as extended techniques, transrectal ultrasound guided prostate biopsy, and others as known to the skilled person. Such samples are primary isolates (in contrast to cultured cells) and may be collected by any suitable means recognized in the art. In some embodiments, the “sample” may be collected by an invasive method, including, but not limited to, surgical biopsy. A sample may contain prostate tumor cells which are isolated by known methods or other appropriate methods as deemed desirable by the skilled practitioner. Isolation methods include, but are not limited to, microdissection, laser capture microdissection (LCM), or laser microdissection (LMD) before use in accordance with the invention. “Expression” and “gene expression” include transcription and/or translation of nucleic acid material. As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

“Detection” includes any means of detecting, including direct and indirect detection of gene expression and changes therein. For example, “detectably less” products may be observed directly or indirectly, and the term indicates any reduction (including the absence of detectable signal). Similarly, “detectably more” product means any increase, whether observed directly or indirectly.

“Prostatectomy” refers to the removal of prostate tissue by a skilled clinician, such as a surgeon. Non-limiting examples include radical prostatectomy; open (traditional) prostatectomy (involving an incision through the perineum); laparoscopic prostatectomy; and robotic (nerve sparing) prostatectomy.

“Gleason score” refers to the grading of a sample of prostate cancer by a trained pathologist according to the Gleason system, which assigns a Gleason score using numbers from 1 to 5 based upon similarities in the cells of a sample of prostate tissue to cancerous tissue or normal prostate tissue. Tissue that looks much like normal prostate tissue is given a score or grade of 1 while a tissue that lacks normal features and the cells seem to be spread haphazardly through the prostate is given a score or grade of 5. Scores, or grades of 2 through 4, inclusive, have features in between these possibilities. But because prostate cancers may have areas with different scores or grades, separate scores or grades are given to the two areas that make up most of the tissue. The two scores or grades are added to yield a Gleason score (or Gleason sum) between 2 and 10.

The expression levels of the marker genes referred to herein correlate clinical outcomes as described herein and therefore may be used as predictors as described herein. The identified genes have been reported to participate in various cellular functions as outlined in http://csbi.ltdk. Helsinki.fi/anduril/tcga-gbm/table4_2.html. In the methods underlying the embodiments of the present invention, these data were correlated with the putative prognostic gene marker PDE4D7 as described in WO2010131194 and WO2010131195, whose contents are herewith incorporated by reference. In the context of the present invention, those molecular pathways that showed significant correlation were selected for down-stream analysis (FIG. 2).

In embodiments of the invention, the marker gene expression pattern (or the PCPI) may be correlated to pre- or post-surgery PSA levels, Gleason score and pT stage. The profile also has the ability to stratify samples with Gleason scores of <=6 and 7 into low and high risk of PSA failure. In multivariate analysis with known prognostic factors, the profile was the only predictor that remains statistically significant (p<0.05). Therefore, detection of the profile at biopsy, or in tissue removed during prostatectomy, allows the distinguishing of indolent from aggressive cancers.

Embodiments of the invention thus relate to the identification and use of gene expression patterns (or profiles or “signatures”) which discriminate between (or are correlated with) prostate cancer survival and recurrence outcomes in a subject. Such patterns may be determined by the methods of the invention by use of a number of reference cell or tissue samples, such as those reviewed by a pathologist of ordinary skill in the pathology of prostate cancer, which reflect prostate cancer cells as opposed to normal or other non-cancerous cells. The outcomes experienced by the subjects from whom the samples may be correlated with expression data to identify patterns that correlate with the outcomes. Because the overall gene expression profile differs from person to person, cancer to cancer, and cancer cell to cancer cell, correlations between certain cells and genes expressed or underexpressed may be made as disclosed herein to identify genes that are capable of discriminating between prostate cancer outcomes.

In embodiments of the present invention, genes with significant correlations to prostate cancer (or pre- or post-prostatectomy) survival, metastasis, or recurrence outcomes are used to discriminate between outcomes. The patterns or indices are capable of predicting the classification of an unknown sample based upon the expression of the genes used for discrimination in the models.

In embodiments of the present invention, marker genes (sequences) expressed in correlation with prostate cancer, or pre- or post-prostatectomy/primary treatment, outcomes provide the ability to focus gene expression analysis to only those genes that contribute to the ability to stratify a subject among different outcomes.

As will be appreciated by those skilled in the art, that the expression patterns (and the PCPI) are highly useful for discriminating between different outcomes in prostate cancer, or pre- or post-prostatectomy, and may be readily performed by the skilled artisan to permit the generation of models as described above to predict the status of an unknown sample based upon the expression levels of those genes.

To determine the (up-regulated or down-regulated) expression levels of genes (expressed sequences) in the practice of the invention, any method known in the art may be utilized. In some embodiments, expression based on detection of RNA which hybridizes to the genes (or probe sequences) identified and disclosed herein is used. This is readily performed by any RNA detection or amplification+detection method known or recognized as equivalent in the art such as, but not limited to, RNA sequencing, reverse transcription-PCR (RT-PCR), real-time PCR, real-time RT-PCR, the methods disclosed in U.S. patent application Ser. No. 10/062,857 (filed on Oct. 25, 2001) as well as U.S. Provisional Patent Applications 60/298,847 (filed Jun. 15, 2001) and 60/257,801 (filed Dec. 22, 2000), and methods to detect the presence, or absence, of RNA stabilizing or destabilizing sequences.

Alternatively, expression based on detection of DNA status may be used. Detection of the DNA of an identified gene as methylated or deleted may be used for genes that have decreased expression in correlation with a particular prostate cancer, or pre- or post-prostatectomy, outcome. This may be readily performed by PCR based methods known in the art, including, but not limited to, Q-PCR.

Conversely, detection of the DNA of an identified gene as amplified may be used for genes that have up-regulated expression in correlation with a particular prostate cancer, or pre- or post-prostatectomy, outcome. This may be readily performed by PCR based, fluorescent in situ hybridization (FISH) and chromosome in situ hybridization (CISH) methods known in the art.

Expression based on detection of a presence, increase, or decrease in protein levels or activity may also be used, e.g. in addition to calculating a PCPI for an individual sample/patient. Detection may be performed by any immunohistochemistry (IHC) based, blood based (especially for secreted proteins), antibody (including autoantibodies against the protein) based, exfoliate cell (from the cancer) based, mass spectroscopy based, and image (including used of labeled ligand) based method known in the art and recognized as appropriate for the detection of the protein. Antibody and image based methods are additionally useful for the localization of tumors after determination of cancer by use of cells obtained by a non-invasive procedure (such as lavage or needle aspiration), where the source of the cancerous cells is not known. A labeled antibody or ligand may be used to localize the carcinoma(s) within a patient.

Embodiments of the present invention relating to the use of a nucleic acid based assay to determine expression are accomplished by immobilization of one or more sequences of the marker genes identified herein as a polynucleotide on a solid support, including, but not limited to, a solid substrate as an array or to beads or bead based technology as known in the art. In some embodiments, the assay is DASL (cDNA-mediated Annealing, Selection, extension and Ligation) assay available from IIlumina. Alternatively, solution based expression assays known in the art may also be used. The immobilized polynucleotide probes may be unique or otherwise specific to the disclosed genes (or expressed sequences) such that the polynucleotides are capable of hybridizing to a DNA or RNA corresponding to the genes (or expressed sequences). These polynucleotides may be the full length of the genes (or expressed sequences) or be probes of shorter length (up to one nucleotide shorter than the full length sequence known in the art by deletion from the 5′ or 3′ end of the sequence) that are optionally minimally interrupted (such as by mismatches or inserted non-complementary basepairs) so that their hybridization with cognate DNA or RNA corresponding to the genes (or expressed sequences) is not affected. In many embodiments, a polynucleotide probe contains sequence from the 3′ end of a disclosed gene or expressed sequence. Polynucleotide probes containing mutations relative to the sequences of the disclosed genes may also be used so long as the presence of the mutations still allows hybridization to produce a detectable signal. The immobilized polynucleotides may be used to determine the state of nucleic acid samples prepared from sample prostate cell(s) for which the outcome of the sample's subject (e.g. patient from whom the sample is obtained) is not known or for confirmation of an outcome that is already assigned to the sample's subject. Without limiting the invention, such a cell may be from a patient with prostate cancer, such as material removed by prostatectomy. The immobilized polynucleotide(s) need only be sufficient to specifically hybridize to the corresponding nucleic acid molecules derived from the sample under suitable conditions. While expression of even a single correlated gene may to able to provide adequate accuracy in discriminating between two prostate cancer outcomes, the invention includes use of expression levels from more than one gene or expressed sequence. In particular, the genes (expressed sequences) of the set of 17, 18, 19, or the set of 57 referred to in the examples section (infra), are used.

In some embodiments, the nucleic acid derived from the sample prostate cancer cell(s) may be preferentially amplified by use of appropriate primers (such as used in PCR) such that only the genes to be analyzed are amplified to reduce contaminating background signals from other expressed genes or sequences in the sample. The size of a PCR amplicon of the invention may be of any size, including at least or about 50, at least or about 100, at least about or 150, at least or about 200, at least or about 250, at least or about 300, at least or about 350, or at least or about 400 consecutive nucleotides, all with inclusion of the portion complementary to the PCR primers used. Of course the PCR may optionally be reverse-transcription coupled PCR (or RT-PCR in the case of RNA starting material) or quantitative PCR, such as real-time PCR, or combinations thereof. Of course RNA from the samples described herein may be prepared and used by means readily known to the skilled person. Alternatively, and where multiple genes are to be analyzed or where very few cells (or one cell) is used, the nucleic acid from the sample may be globally amplified before hybridization to immobilized polynucleotide probes, such as on an array or microarray. Of course RNA, or the cDNA counterpart thereof may be directly labeled and used, without amplification, by methods known in the art. A preferred embodiment of the methods for detection of the herein described marker genes is RNA sequencing.

The invention provides a more objective set of criteria, in the form of gene expression profiles of a discrete set of marker genes, to discriminate (or delineate) between prostate cancer, or pre- or post-prostatectomy, outcomes, and allows for the prediction of a predisposition for aggressive versus indolent disease. In some embodiments, the assays are used to discriminate between better and poorer outcomes within about 6 months, 1 year, 2 years, 3 years, 4 years, 5 years, 7 years, 10 years, 15 years or even later.

While better and poorer cancer recurrence, metastasis and/or survival outcomes may be defined relatively in comparison to each other, a “better” outcome or a “good prognosis” may be viewed as one that is better than a 50% chance of cancer recurrence and/or 50% chance of survival after about 60 months post surgical intervention to remove prostate cancer tumor(s). A “better” outcome or a “good prognosis” may also be a better than about 60%, about 70%, about 80% or about 90% cancer recurrence and/or chance of survival about 60 months post surgical intervention. A “poorer” outcome/prognosis may be viewed as a 50% or more chance of cancer recurrence and/or less than 50% chance of survival after about 60 months post surgical intervention to remove prostate cancer tumor(s). The PCPI and the methods according to the invention help classifying patients into those that have the above prognosis.

The disclosed methods may also be used with solid tissue material. For example, a solid biopsy may be collected and prepared for visualization followed by determination of expression of two or more genes or expressed sequences identified herein to determine the prostate cancer, or pre- or post-prostatectomy, outcome. One means is by use of in situ hybridization with polynucleotide identifying probe(s) for assaying expression of said gene(s).

In some embodiments, the detection of gene expression from the samples may be by use of a single microarray able to assay gene expression from some or all genes disclosed herein for convenience and accuracy.

Other uses of the invention include providing the ability to identify prostate cancer cell samples as correlated with particular prostate cancer survival or recurrence outcomes for further research or study. This provides a particular advantage in many contexts requiring the identification of cells based on objective genetic or molecular criteria.

The materials for use in the methods of the present invention are ideally suited for preparation of kits produced in accordance with well known procedures. The invention thus provides kits comprising agents for the detection of expression of the disclosed genes and sequences for identifying prostate cancer, or pre- or post-prostatectomy, outcomes. Such kits optionally comprise the agent with an identifying description or label or instructions relating to their use in the methods of the present invention, is provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, RGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

The methods provided by the invention may also be automated in whole or in part. All aspects of the invention may also be practiced such that they consist essentially of a subset of the disclosed genes to the exclusion of material irrelevant to the identification of prostate cancer recurrence, metastasis, and/or survival outcomes via a cell containing sample.

In the context of the methods of the present invention, the PCPI is used to diagnose prostate cancer in patients, identify prostate cancer in patients, and to establish a prognosis for prostate cancer patients, to determine the predisposition to develop aggressive versus indolent disease, and to stratify prostate cancer patients so that a decision can be made by the treating physician on how, if at all, a patient should be treated. The PCPI can be used as sole parameter for the above purposes or it can be used in combination with other known diagnostic, prognostic, or stratification methods to provide further support for the physician.

The term “diagnosing prostate cancer” as used herein means that a patient or individual may be considered to be suffering from a prostate cancer, when the expression level of the GOI'(s) of the present invention is either up-regulated or down-regulated, compared to a control level as defined herein above, preferably if compared to the normal control level as defined herein above so that the PCPI is significantly higher than/different from that found in a control tissue. The term “diagnosing” also refers to the conclusion reached through that comparison process.

In another embodiment of the present invention, the diagnosis may be combined with the elucidation of additional cancer biomarker expression levels, in particular prostate cancer biomarkers. Several particular prostate cancer biomarkers would be known to the person skilled in the art. For example, the expression of biomarkers like PSA may be tested.

A prostate cancer may be considered as being diagnosed when the expression level of the GOI's of the present invention is up-regulated or down-regulated, compared to the normal control level as defined herein above and when the PCPI is (significantly higher than the one obtained with control material).

In a further preferred embodiment a prostate cancer may considered as being diagnosed or a predisposition to develop aggressive vs. indolent disease be determined, if the expression level of the GOI's referred to herein, is up-regulated or down-regulated by a value of between 20% to 80%, preferably by a value of 30%, 40%, 50%, 60% or 70% in a test sample in comparison to a control level, preferably a normalized control level. The control level may either be a normal control level or a cancerous control level, as defined herein above. In a particularly preferred embodiment a prostate cancer may be considered as being diagnosed or a predisposition to develop aggressive vs. indolent disease if the expression level, as defined herein above, is up-regulated or down-regulated by a factor of 1.5- to 10-fold, preferably by a factor of 2- to 5-fold in a test sample in comparison to a control level, in particular a healthy tissue or a benign prostate tumor or very preferably in comparison to prostate cancer tissue of the same patient at initial diagnosis.

The term “detecting prostate cancer” as used herein means that the presence of a prostate cancer disease or disorder in an organism may be determined or that such a disease or disorder may be identified in an organism. The determination or identification of a prostate cancer disease or disorder may be accomplished by a comparison of the expression level of the GOI's of the present invention and the normal control level as defined herein above, or by determining the PCPI. A prostate cancer may be detected when the expression level of the GOI's is up-regulated or down-regulated in comparison to the normal control level as defined herein above. In a preferred embodiment of the present invention a prostate cancer may be detected if the expression level of the GOI's is similar to an expression level of an established, e.g. independently established, prostate cancer cell or cell line, e.g. a prostate cancer cell line, or a cell line as mentioned herein above.

The terms “monitoring prostate cancer” and “identifying a subject with a predisposition of prostate cancer that is susceptible to disease progression” (i.e. identification of aggressive disease versus indolent disease), as used herein relates to the accompaniment of a diagnosed or detected prostate cancer disease or disorder, e.g. during or after a treatment procedure or during or after a certain period of time, typically during 2 months, 3 months, 4 months, 6 months, 1 year, 2 years, 3 years, 5 years, 10 years, or any other period of time during or after the start or end of the primary treatment. The term “accompaniment” means that states of disease or the PCPI as defined herein above and, in particular, changes of these states of disease or PCPI may be detected by comparing the expression levels of the GOI's of the present invention in a sample to a normal control level as defined herein above, preferably a control expression level derived from a benign tumor control or a healthy control or to the expression level of an established, e.g. independently established, prostate cancer cell or cell line, e.g. a prostate cancer cell line, or a cell line in any type of periodical time segment, e.g. every week, every 2 weeks, every month, every 2, 3, 4, 5, 6, 7, 8, 9, 10, 11 or 12 month, every 1.5 year, every 2, 3, 4, 5, 6, 7, 8,9 or 10 years, during any period of time, e.g. during 2 weeks, 3 weeks, 1, 2, 3, 4, 5, 6, 7, 8, 9, 10, 11, 12 months, 1.5, 2, 3, 4, 5, 6, 7, 8, 9, 10, 15 or 20 years, respectively. The established, e.g. independently established, prostate cancer cell or cell line giving rise to an additional control level may be derived from samples corresponding to different stages of cancer development, e.g. stages 0 and I to IV of the TNM classification system. In a preferred embodiment of the present invention the term relates to the accompaniment of a diagnosed prostate cancer, e.g. a malignant, hormone- sensitive prostate cancer. The monitoring may also include the detection of the expression of additional genes or genetic elements, e.g. housekeeping genes like GAPDH or PBGD. The present invention also relates to methods of monitoring patients with a view to identify changes in the herein disclosed signature genes using the PCPI as disclosed throughout the specification in order to identify those patients likely developing aggressive prostate cancer. These patients will then be treated as described throughout the specification.

The term “prognosticating (aggressive) prostate cancer” as used herein refers to the prediction of the course or outcome of a diagnosed or detected aggressive prostate cancer, e.g. during a certain period of time, during a treatment or after a treatment. The term also refers to a determination of chance of survival or recovery from the disease, as well as to a prediction of the expected survival time of a patient. A prognosis may, specifically, involve establishing the likelihood for survival of a patient during a period of time into the future, such as 6 months, 1 year, 2 years, 3 years, 5 years, 10 years or any other period of time.

The terms “progression towards prostate cancer” and “progression towards aggressive (versus indolent) prostate cancer” as used herein relates to a switch between different stages of cancer development, e.g. stages 0 and Ito IV of the TNM classification, or any other stage or sub-stage, starting from a healthy condition up to malignant, hormone-sensitive or malignant hormone-resistant prostate cancer. Typically such switches are accompanied by a modification of the expression level of marker gene(s) or GOI's and a different PCPI as used herein, in a test sample in comparison to a previous test sample from the same individual, e.g. in comparison to a sample derived from a benign prostate tumor control or a healthy tissue control. A progression towards aggressive prostate cancer may be considered as being detected or diagnosed if the expression level of GOI's used for calculating the PCPI, as defined herein above, is different, e.g. by a value of between 3% to 50%, preferably by a value of 10%, 15%, 20% or 25% in a test sample in comparison to a previous PCPI calculated from the gene expression values in a test sample from the same individual. The modification may be detected over any period of time, preferably over 1, 2, 3, 4, 5, 6, 7, 8, 9, 10, 11, 12 months, 1.5, 2, 3, 4, 5, 6, 7, 8, 9, 10, 15 or 20 years, i.e. the value indicated above may be calculated by comparing the expression level of marker gene(s) or GOI's or the PCPI at a first point in time and at a second point in time after the above indicated period of time.

In a particularly preferred embodiment of the present invention the term “progression towards prostate cancer” relates to a switch from a healthy state or a benign prostate tumor state to a malignant prostate cancer state. A progression from a healthy state to a prostate cancer state may be considered as being detected or diagnosed if the PCPI, as defined herein above, is higher by a value of between 20% to 50%, preferably by a value of 20%, 25%, 30% or 35% in a test sample in comparison to a previous test sample from the same individual, which was diagnosed as being healthy. Alternatively, for the comparison test samples from other individuals may be used, e.g. test samples of healthy individuals. Also envisaged is the use of available database information on the expression or the employment of cancer cell collection samples etc.

A progression from a benign prostate tumor state to a malignant prostate cancer state may be considered as being detected or diagnosed if the PCPI, as defined herein above, is higher by a value of between 3% to 30%, preferably by a value of 5%, 10%, 15%, or 20% in a test sample in comparison to a previous test sample from the same individual, which has been diagnosed as suffering from a benign prostate tumor. Alternatively, for the comparison test samples from other individuals may be used, e.g. test samples of individuals diagnosed to suffer from benign prostate tumors. Also envisaged is the use of available database information on the expression or the employment of cancer cell collection samples etc.

The modification may be detected over any period of time, preferably over 1, 2, 3, 4, 5, 6, 7, 8, 9, 10, 11, 12 months, 1.5, 2, 3, 4, 5, 6, 7, 8, 9, 10, 15 or 20 years, i.e. the value indicated above may be calculated by comparing the expression level of marker gene(s) or GOI's at a first point in time and at a second point in time after the above indicated period of time.

In a further embodiment the present invention relates to the diagnosis and detection of a predisposition for developing prostate cancer. A “predisposition for developing (aggressive) prostate cancer” in the context of the present invention is a state of risk of developing (aggressive) prostate cancer. Preferably a predisposition for developing aggressive prostate cancer may be present in cases in which the GOI's expression level or the PCPI defined herein above is significantly different from a control level as defined herein, i.e. a reference expression level or reference PCPI derived from tissues or samples of a patient which are evidently healthy, or from a sample or tissues of a patient obtained before initial diagnosis of prostate cancer, so that also the PCPI calculated on the basis of the gene expression in the cancerous tissues is higher than the PCPI obtained for the control sample. Thus, a predisposition for aggressive prostate cancer may be considered as being diagnosed or detected if the above depicted situation is observed.

Generally, and also in the establishment of the PCPI, the difference between the expression levels of a test biological sample and a control level can be normalized to the expression level of further control nucleic acids, e.g. housekeeping genes whose expression levels are known not to differ depending on the cancerous or noncancerous state of the cell. Exemplary control genes include inter alfa β-actin, glycerinaldehyde 3-phosphate dehydrogenase (GAPDH), porphobilinogen deanimase (PBGD) and the like.

In the context of the present invention, the terms “diagnosing” and “prognosticating” are also intended to encompass predictions and likelihood analyses. The PCPI as a parameter may accordingly be used clinically in making decisions concerning treatment modalities, including therapeutic intervention or diagnostic criteria such as a surveillance for the disease. According to the present invention, an intermediate result for examining the condition of a patient may be provided. Such intermediate result may be combined with additional information to assist a doctor, nurse, or other practitioner to diagnose that a patient suffers from the disease. Alternatively, the present invention may be used to detect cancerous cells in a patient-derived tissue, and provide a doctor with useful information to diagnose that the patient suffers from the disease.

A patient or individual to be diagnosed, monitored or in which a prostate cancer, a progression towards aggressive versus indolent prostate cancer or a predisposition for aggressive versus indolent prostate cancer is to be detected or prognosticated according to the present invention is an animal, preferably a mammal, more preferably a human being. The use of molecular imaging tools as known to the person skilled in the art, e.g. magnetic resonance imaging (MRI) and/or magnetic photon resonance imaging (MPI) technology and/or multi-parametric MRI (mpMRI) technology in the in addition to the PCPI as a marker for diagnosing, detecting, monitoring or prognosticating prostate cancer of the progression towards aggressive versus indolent prostate cancer.

In a further aspect the present invention relates to a composition for diagnosing, detecting, monitoring or prognosticating prostate cancer or the progression towards prostate cancer or a predisposition for prostate cancer in an individual. The composition according to the present invention may comprise a nucleic acid affinity ligand for the GOI expression product(s).

The term “nucleic acid affinity ligand for the GOI expression product” as used herein refers to a nucleic acid molecule being able to specifically bind to a GOI transcript. The nucleic acid affinity ligand may also be able to specifically bind to a nucleic acid sequence being at least 90%, 91%, 92%, 93%, 94%, 95%, 96%, 97%, 98% or 99% identical to GOI sequences, or to any fragments of said sequences.

The term “expression product” as used herein refers to a transcript of a GOI or an mRNA molecule generated by the expression of the GOI. More preferably, the term relates to a processed transcript as defined herein above.

The composition of the present invention may, for example, comprise a set of oligonucleotides specific for the GOI expression products and/or a probe specific for the GOI expression products. The term “oligonucleotide specific for the GOI expression products” as used herein refers to a nucleotide sequence which is complementary to the sense- or antisense-strand of a GOI.

The oligonucleotide may have any suitable length and sequence known to the person skilled in the art, as derivable from the sequence of the GOI or its complement. Typically, the oligonucleotide may have a length of between 8 and 60 nucleotides, preferably of between 10 and 35 nucleotides, more preferably a length of 11, 12, 13, 14, 15, 16, 17, 18, 19, 20, 21, 22, 23, 24, 25, 26, 27, 28, 29, 30, 31, 32 or 33 nucleotides. Oligonucleotide sequences, for instance, oligonucleotides usable as a forward and reverse primers specific for the GOI expression products may be defined with the help of software tools known to the person skilled in the art.

The term “probe specific for the GOI expression product” as used herein means a nucleotide sequence which is complementary to the sense- or antisense-strand of the herein described GOI's.

The probe may have any suitable length and sequence known to the person skilled in the art, as derivable from the sequence of the herein described GOI's. Typically, the probe may have a length of between 6 and 300 nucleotides, preferably of between 15 and 60 nucleotides, more preferably a length of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. Probe sequences specific for the GOI's expression product may be defined with the help of software tools known to the person skilled in the art.

A nucleic affinity ligand, as described herein above, may be labeled with various markers or may be detected by a secondary affinity ligand, labeled with various markers, to allow detection, visualization and/or quantification. This can be accomplished by using any suitable labels, which can be conjugated to the affinity ligand capable of interaction with the expression product or to any secondary affinity ligand, using any suitable technique or methods known to the person skilled in the art. The term “secondary affinity ligand” refers to a molecule which is capable of binding to the affinity ligand as defined herein above (i.e. a “primary affinity ligand” if used in the context of a system with two interacting affinity ligands). The binding interaction is preferably a specific binding.

Examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bio luminescent proteins (e.g. luciferin, luciferase), haptens (e.g. biotin).

In embodiments of the present invention, nucleic affinity ligands to be used as probes, in particular a probe specific for the GOI expression products as defined herein above, may be labeled with a fluorescent label like 6-FAM, HEX, TET, ROX, Cy3, Cy5, Texas Red or Rhodamine, and/or at the same time with a quenching label like TAMRA, Dabcyl, Black Hole Quencher, BHQ-I or BHQ-2. A variety of other useful fluorescents and chromophores are described in Stryer, 1968, Science, 162:526-533. Affinity ligands may also be labeled with enzymes (e.g. horseradish peroxidase, alkaline phosphatase, beta-lactamase), radioisotopes (e.g. 3H, 14C, 32P, 33P, 35S, 1251) or particles (e.g. gold). The different types of labels may be conjugated to an affinity ligand using various chemistries, e.g. the amine reaction or the thiol reaction. However, other reactive groups than amines and thiols can also be used, e.g. aldehydes, carboxylic acids and glutamine.

In a specific embodiment of the present invention a composition may additionally comprise accessory ingredients like PCR buffers, dNTPs, a polymerase, ions like bivalent cations or monovalent cations, hybridization solutions, detection dyes and any other suitable compound or liquid necessary for the performance of a detection as defined herein above, which is known to the person skilled in the art.

In another aspect the present invention relates to the use of a nucleic acid for the GOI expression product, as defined herein above, for the preparation of a composition for diagnosing, detecting, monitoring or prognosticating prostate cancer or the progression towards aggressive versus indolent prostate cancer or a predisposition for aggressive versus indolent disease in an individual, as described herein above.

In a preferred embodiment the present invention relates to the use of a set of oligonucleotides specific for the GOI expression products and/or a probe specific for the expression products, as defined herein above, for the preparation of a composition for diagnosing, detecting, monitoring or prognosticating prostate cancer or the progression towards aggressive versus indolent prostate cancer or a predisposition for aggressive versus indolent disease in an individual, as described herein above.

In a preferred embodiment of the present invention a composition as defined herein above is a diagnostic composition.

In another aspect the present invention relates to a diagnostic kit for detecting, diagnosing, monitoring or prognosticating prostate cancer or the progression towards prostate cancer or a predisposition for aggressive prostate cancer, comprising a set of oligonucleotides specific for the GOI expression products, a probe specific for the GOI expression products.

Typically, the diagnostic kit of the present invention contains one or more agents allowing the specific detection of marker gene(s) or GOI's as defined herein above. The agents or ingredients of a diagnostic kit may, according to the present invention, be comprised in one or more containers or separate entities. The nature of the agents is determined by the method of detection for which the kit is intended.

Furthermore, the kit may comprise an amount of a known nucleic acid molecule, which can be used for a calibration of the kit or as an internal control. Typically, a diagnostic kit for the detection of marker gene(s) or GOI's expression products may comprise accessory ingredients like a PCR buffers, dNTPs, a polymerase, ions like bivalent cations or monovalent cations, hybridization solutions etc. Such ingredients are known to the person skilled in the art and may vary depending on the detection method carried out. Additionally, the kit may comprise an instruction leaflet and/or may provide information as to the relevance of the obtained results.

In other aspects the present invention relates to methods for detecting, diagnosing, monitoring or prognosticating prostate cancer or the progression towards aggressive versus indolent prostate cancer, e.g. in an individual, comprising at least the step of determining the level of marker gene(s) or GOI's in a sample. The term “determining the level of marker gene(s) or GOI's” refers to the determination of the presence or amount of marker gene(s) or GOI's expression products. The term “level of marker gene(s) or GOI's” thus means the presence or amount of marker gene(s) or GOI's expression products, e.g. transcript(s), and/or the determination of the presence or amount of marker gene(s) or GOI's. The determination of the presence or amount of marker gene(s) or GOI's expression products, may be accomplished by any means known in the art.

In a preferred embodiment of the present invention the determination of the presence or amount of marker gene(s) or GOI's expression products is accomplished by the measurement of nucleic acid. Thus, the expression level(s) may be determined by a method involving the detection of an mRNA encoded by the gene.

For example, the measurement of the nucleic acid level of marker gene(s) or GOI's expression may be assessed by purification of nucleic acid molecules (e.g. RNA or cDNA) obtained from the sample, followed by hybridization with specific oligonucleotide probes as defined herein above. Comparison of expression levels may be accomplished visually or by means of an appropriate device. Methods for the detection of mRNA or expression products are known to the person skilled in the art.

Alternatively, the nucleic acid level of marker gene(s) or GOI's expression may be detected in a DNA array or microarray approach. Typically, sample nucleic acids derived from patients to be tested are processed and labeled, preferably with a fluorescent label. Subsequently, such nucleic acid molecules may be used in a hybridization approach with immobilized capture probes corresponding to the marker genes of the present invention. Suitable means for carrying out microarray analyses are known to the person skilled in the art.

In a standard setup a DNA array or microarray comprises immobilized high-density probes to detect a number of genes. The probes on the array are complementary to one or more parts of the sequence of the marker genes. Typically, cDNAs, PCR products, and oligonucleotides are useful as probes.

A DNA array- or microarray-based detection method typically comprises the following steps: (1) Isolating mRNA from a sample and optionally converting the mRNA to cDNA, and subsequently labeling this RNA or cDNA. Methods for isolating RNA, converting it into cDNA and for labeling nucleic acids are described in manuals for micro array technology. (2) Hybridizing the nucleic acids from step 1 with probes for the marker genes. The nucleic acids from a sample can be labeled with a dye, such as the fluorescent dyes Cy3 (red) or Cy5 (blue). Generally a control sample is labeled with a different dye. (3) Detecting the hybridization of the nucleic acids from the sample with the probes and determining at least qualitatively, and more particularly quantitatively, the amounts of mRNA in the sample for marker genes investigated. The difference in the expression level between sample and control can be estimated based on a difference in the signal intensity. These can be measured and analyzed by appropriate software such as, but not limited to the software provided for example by Affymetrix.

There is no limitation on the number of probes corresponding to the marker genes used, which are spotted on a DNA array. Also, a marker gene can be represented by two or more probes, the probes hybridizing to different parts of a gene. Probes are designed for each selected marker gene. Such a probe is typically an oligonucleotide comprising 5-50 nucleotide residues. Longer DNAs can be synthesized by PCR or chemically. Methods for synthesizing such oligonucleotides and applying them on a substrate are well known in the field of micro-arrays. Genes other than the marker genes may be also spotted on the DNA array. For example, a probe for a gene whose expression level is not significantly altered may be spotted on the DNA array to normalize assay results or to compare assay results of multiple arrays or different assays.

Alternatively, the nucleic acid level of marker gene(s) or GOI's expression may be detected in a quantitative RT-PCR approach, preferably in a real-time PCR approach following the reverse transcription transcripts of interest. Typically, as first step, a transcript is reverse transcribed into a cDNA molecule according to any suitable method known to the person skilled in the art. A quantitative or real-time PCR approach may subsequently be carried out based on a first DNA strand obtained as described above.

Preferably, Taqman or Molecular Beacon probes as principal FRET-based probes of this type may be used for quantitative PCR detection. In both cases, the probes, serve as internal probes which are used in conjunction with a pair of opposing primers that flank the target region of interest, preferably a set of marker gene(s) specific oligonucleotides as defined herein above. Upon amplification of a target segment, the probe may selectively bind to the products at an identifying sequence in between the primer sites, thereby causing increases in FRET signaling relative to increases in target frequency.

Preferably, a Taqman probe to be used for a quantitative PCR approach according to the present invention may comprises a specific oligonucleotide as defined above of about 22 to 30 bases that is labeled on both ends with a FRET pair. Typically, the 5′ end will have a shorter wavelength fluorophore such as fluorescein (e.g. FAM) and the 3′ end is commonly labeled with a longer wavelength fluorescent quencher (e.g. TAMRA) or a non-fluorescent quencher compound (e.g. Black Hole Quencher). It is preferred that the probes to be used for quantitative PCR, in particular probes as defined herein above, have no guanine (G) at the 5′ end adjacent to the reporter dye in order to avoid quenching of the reporter fluorescence after the probe is degraded.

A Molecular Beacon probe to be used for a quantitative PCR approach according to the present invention preferably uses FRET interactions to detect and quantify a PCR product, with each probe having a 5′ fluorescent-labeled end and a 3′ quencher-labeled end. This hairpin or stem-loop configuration of the probe structure comprises preferably a stem with two short self-binding ends and a loop with a long internal target-specific region of about 20 to 30 bases.

Alternative detection mechanisms which may also be employed in the context of the present invention are directed to a probe fabricated with only a loop structure and without a short complementary stem region. An alternative FRET -based approach for quantitative PCR which may also be used in the context of the present invention is based on the use of two hybridization probes that bind to adjacent sites on the target wherein the first probe has a fluorescent donor label at the 3′ end and the second probe has a fluorescent acceptor label at its 5′ end.

In yet another embodiment as a further, additional step a decision on the presence or stage of cancer or the progression of cancer may be based on the results of a comparison step. A malignant, hormone-sensitive prostate cancer may be diagnosed or prognosticated or a progression towards (aggressive) prostate cancer may be diagnosed or prognosticated in said method according to the corresponding definitions provided herein above in the context of marker gene(s) or GOI's as marker for malignant prostate cancer.

In another embodiment the present invention relates to a method for detecting, diagnosing, monitoring or prognosticating (aggressive) prostate cancer or the progression towards (aggressive) prostate cancer comprising at least the steps of:

(a) testing in at least one sample obtained from at least one individual suspected to suffer from prostate cancer for expression level of the GOI expression products;

(b) testing in at least one control sample for the expression level of the GOI expression product;

(c) determining the difference in the expression of steps (a) and (b); and

(d) deciding on the presence or stage of prostate cancer or the progression towards aggressive prostate cancer based on the results obtained in step (c).

In one embodiment, steps a), b), c) and/or d) of this method of diagnosis may be performed outside the human or animal body, e.g. in samples obtained from a patient or individual.

In another aspect the present invention relates to a method for diagnosing, monitoring or prognosticating, e.g. aggressive, prostate cancer or the progression towards aggressive prostate cancer, wherein said method discriminates between a benign and aggressive prostate cancer, comprising the steps of

(a) determining the level of marker gene(s) or GOI's,

(b) determining the level of expression of a reference gene in a sample;

(c) normalizing the measured expression level of marker gene(s) or GOI's to the expression of the reference gene; and

(d) comparing the normalized expression level with a predetermined cutoff value chosen to exclude benign prostate tumor, wherein a normalized expression level above the cutoff value is indicative of a aggressive prostate cancer, wherein said cutoff value is between −2 and +2, preferably about 0.

Expression results may be normalized according to any suitable method known to the person skilled in the art. Typically, such tests or corresponding formula, which would be known to the person skilled in the art, would be used to standardize expression data to enable differentiation between real variations in gene expression levels and variations due to the measurement processes. For microarrays, the Robust Multi-array Average (RMA) may be used as normalization approach.

In a specific embodiment the normalized values are generated by applying the following:

N(Cq_{gene of interest})=Mean (Cq_{ref gene})−(Cq_{gene of interest})

Where N(Cq_{gene of interest}) is normalized gene expression value for selected genes of interest; where Mean(Cq_{ref gene}) is the arithmetic mean of the PCR Cq values of the selected combination of reference genes; where (Cq_{gene of interest}) is the PCR Cq value of the gene of interest.

In a specific embodiment the normalized values are generated by applying the following:

N(Cq_{gene of interest})=Mean(Cq_{ref gene})−(Cq_{gene of interest})

Where N(Cq_{gene of interest}) is normalized gene expression value for selected genes of interest; where Mean(Cq_{ref gene}) is the arithmetic mean of the PCR Cq values of the of at least two reference genes selected from TBP, HPRT1, ACTB, RPLP0, POLR2A, B2M, PUM1, K-ALPHA-1 and ALAS-1; where (Cq_{gene of interest}) is the PCR Cq value of the gene of interest.

In a specific embodiment the normalized values are generated by applying the following:

N(Cq_{gene of interest})=Mean(Cq_{ref gene})−(Cq_{gene of interest})

Where N(Cq_{gene of interest}) is normalized gene expression value for selected genes of interest; where Mean(Cq_{ref gene}) is the arithmetic mean of the PCR Cq values of the reference genes TBP, HPRT1, ACTB and RPLP0; where (Cq_{gene of interest}) is the PCR Cq value of the gene of interest.

In a specific embodiment the normalized values are generated by applying the following:

N(Cq_{gene of interest})=Mean(Cq_{ref gene})−(Cq_{gene of interest})

Where N(Cq_{gene of interest}) is normalized gene expression value for selected genes of interest; where Mean(Cq_{ref gene}) is the arithmetic mean of the PCR Cq values of the of the reference genes TBP, HPRT1, PUM1, K-ALPHA-1 and ALAS-1 where (Cq_{gene of interest}) is the PCR Cq value of the gene of interest.

Exemplary reference genes include inter alia Homo sapiens TATA box binding protein (TBP), Homo sapiens hypoxanthine phosphoribosyltransferase 1 (HPRT1), Homo sapiens actin, beta, mRNA (ACTB), Homo sapiens 60S acidic ribosomal phosphoprotein PO mRNA (RPLP0), Homo sapiens pumilio RNA-Binding Family Member (PUM1), Polymerase (RNA) II (DNA Directed) Polypeptide A, 220kDa (POLR2A), Beta-2-Microglobulin (B2M), Tubulin-Alpha-1b (K-ALPHA-1), Aminolevulinate-Delta-Synthase (ALAS-1).

In a preferred embodiment a combination of reference genes is selected from TATA box binding protein (TBP), Homo sapiens hypoxanthine phosphoribosyltransferase 1 (HPRT1), Homo sapiens actin, beta, mRNA (ACTB), Homo sapiens 60S acidic ribosomal phosphoprotein PO mRNA (RPLP0), Homo sapiens pumilio RNA-Binding Family Member (PUM1), Polymerase (RNA) II (DNA Directed) Polypeptide A, 220kDa (POLR2A), Beta-2-Microglobulin (B2M), Tubulin-Alpha-1b (K-ALPHA-1), Aminolevulinate-Delta-Synthase (ALAS-1).

In a specific embodiment the expression level of the signature gene is normalized to the expression of a reference gene, wherein the reference gene is preferably a housekeeping gene and wherein the housekeeping gene is preferably TBP, HPRT1, ACTB, RPLP0, PUM1, POLR2A or B2M.

In another preferred embodiment the combination of reference genes comprises TBP, HPRT1 and at least one, at least two or at least three additional reference genes selected from the group comprising ACTB, RPLP0, PUM1, POLR2A, B2M, K-ALPHA-1 (TUBA1B) or ALAS-1.

In another preferred embodiment the combination of reference genes comprises TBP, HPRT1 and at least two additional reference genes selected from the group comprising ACTB, RPLP0, PUM1, K-ALPHA-1 (TUBA1B) or ALAS-1.

A particularly preferred combination is TBP, HPRT1, ACTB, RPLP0. Another particularly preferred combination is TBP, HPRT1, PUM1, K-ALPHA-1, ALAS-1.

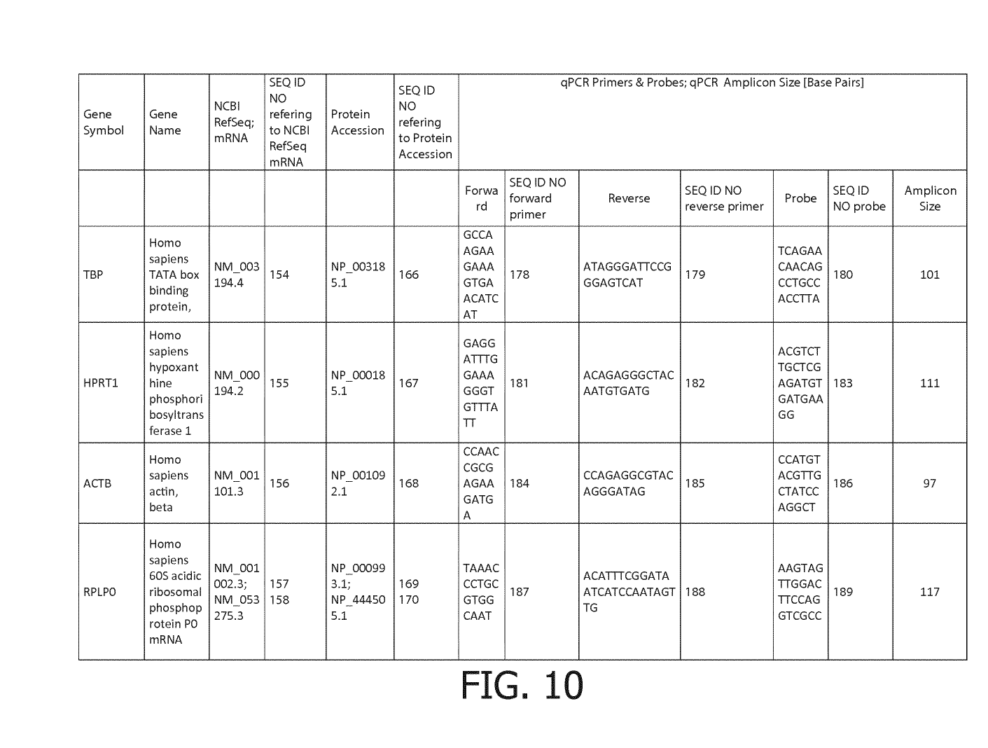

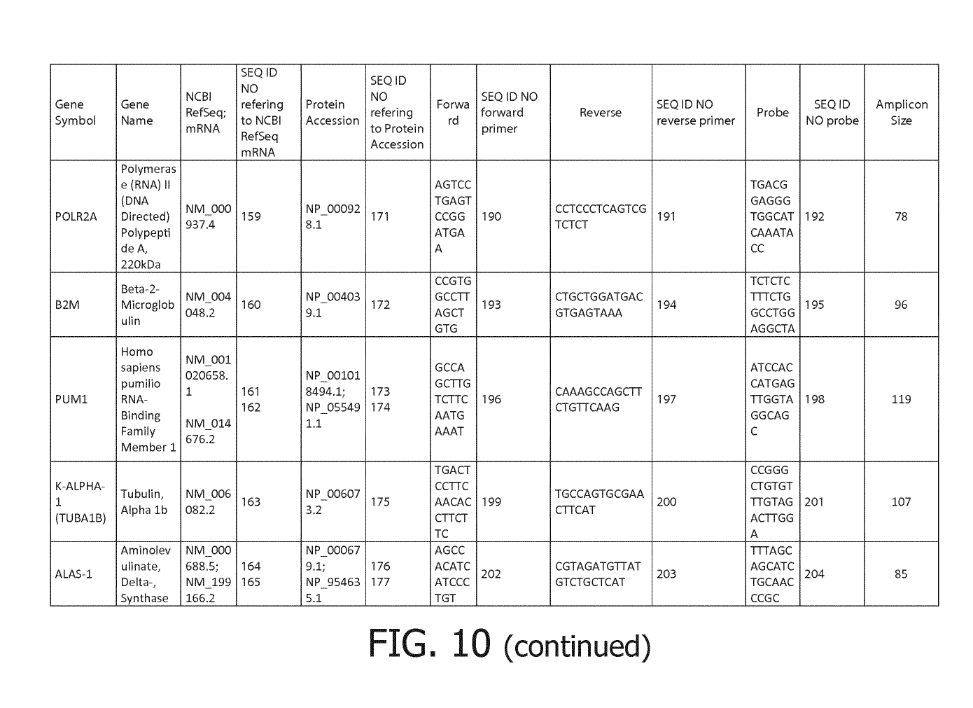

A detailed description of the reference genes including their Transcript ID (NCBI RefSeq) and the SEQ ID NO of the corresponding nucleotide sequence, the protein ID (protein accession) and the SEQ ID NO of the corresponding amino acid sequence is disclosed in FIG. 10. Further FIG. 10 discloses for each reference gene a forward primer, reverse primer for the generation of an amplicon which is specific for the reference gene and a probe sequence that specifically binds to the amplicon. The expression level of marker gene(s) or GOI's may be determined on the nucleic acid, as described herein above. Preferred is the determination of the expression level of marker gene(s) or GOI's transcript(s). In addition the level of a house-keeping gene in sample may be determined.

The term “reference gene” as used herein refers to any suitable gene, e.g. to any steadily expressed and continuously detectable gene, gene product, expression product, protein or protein variant in the organism of choice.

The expression may be preferably be carried out in the same sample, i.e. the level of marker gene(s) or GOI's and of the reference gene is determined in the same sample. If the testing is carried out in the same sample, a single detection or a multiplex detection approach as described herein may be performed. For the performance of the multiplex detection the concentration of primers and/or probe oligonucleotides may be modified. Furthermore, the concentration and presence of further ingredients like buffers, ions etc. may be modified, e.g. up-regulated or down-regulated or decreased in comparison to manufacturers' indications.

Furthermore, expression results may be compared to already known results from reference cases or databases. The comparison may additionally include a normalization procedure in order to improve the statistical relevance of the results.

Expression results may be normalized according to any suitable method known to the person skilled in the art, e.g. according to normalization statistical methods like the standard score, Student's T-test, studentized residual test, standardized moment text, or coefficient variation test. Typically, such tests or corresponding formula, which would be known to the person skilled in the art, would be used to standardize expression data to enable differentiation between real variations in gene expression levels and variations due to the measurement processes.

Based on the expression results obtained in steps (a) and (b) and/or the normalized results obtained in step (c) a comparison with a cutoff value for GOI expression levels may be carried out. The cutoff value above which the expression level of marker gene(s) or GOI's is indicative of an aggressive prostate cancer, thereby excluding benign prostate tumor forms or healthy situations or healthy tissue forms, is between about −3 and +3, −3 and +2.75, −3 and +2.5, −3 and +2.25, −3 and +2, −3 and +1.75, −3 and +1.5, −3 and +1.25, −3 and +1, −3 and +0.75, −3 and +0.5, −3 and +0.25, −3 and 0, −2.75 and +3, −2.5 and +3, −2.25 and +3, −2 and +3, −1.75 and +3, −1.5 and +3, −1.25 and +3, −1 and +3, −0.75 and +3, −0.5 and +3, −0.25 and +3, or 0 and +3. More preferred is a cutoff value of about 0, e.g. 0.9, 0.8, 0.7, 0.6, 0.5, 0.4, 0.3, 0.2, 0.1 or −0.9, −0.8, −0.7, −0.6, −0.5, −0.4, −0.3, −0.2, or −0.1.

If the measured and/or normalized expression levels is below the indicated cutoff value this may be seen as an indication that the individual is either healthy with respect to prostate tumors or suffers only from benign prostate tumors, but not from aggressive prostate cancer.

In another aspect the present invention relates to a method of data acquisition comprising at least the steps of:

(a) testing in an individual for expression of marker gene(s) or GOI's; and

(b) comparing the expression as determined in step (a) to a control level. The testing for expression of marker gene(s) or GOI's may be carried out according to steps as defined herein above. Preferably the testing may be carried out as measurement of nucleic acid of marker gene(s) or GOI's. The testing may be carried out in an individual, i.e. in vivo, or outside the individual, i.e. ex vivo or in vitro. The term “control level” as used in the context of the method of data acquisition refers to the expression of the marker genes referred to herein or other suitable markers in a normal control or a cancerous control, as defined herein above. The status, nature, amount and condition of the control level may be adjusted according to the necessities. Preferably a normal, healthy control level may be used. A comparison of the expression to a control level may be carried out according to any suitable method of assessing, calculating, evaluating or processing of data and particularly aims at the detection of differences between two data sets. A statistical evaluation of the significance of the difference may further be carried out. Suitable statistical methods are known to the person skilled in the art. Obtained data and information may be stored, accumulated or processed by suitable informatics or computer methods or tools known to the person skilled in the art and/or be presented in an appropriate manner in order to allow the practitioner to use the data for one or more subsequent deduction or conclusion steps.

In addition the level of a reference gene as defined herein above in a sample may be determined. Alternatively, the determination of the reference gene may be carried out with any other suitable agent or be combined with the detection of the presence or amount of nucleic acids as described herein.

In a further aspect the present invention relates to a method of identifying an individual for eligibility for prostate cancer therapy comprising:

(a) testing in a sample obtained from an individual for the expression of marker gene(s) or GOI's;

(b) testing in said sample for the expression of a reference gene and/or testing in a control sample for the expression of marker gene(s) or GOI's;

(c) classifying the levels of expression of step (a) relative to levels of step (b); and calculating respective PCPI's,

(d) identifying the individual as eligible to receive a prostate cancer therapy where the individual's sample is classified as having an up-regulated or down-regulated level of marker gene(s) or GOI's expression or an PCPI that is indicative of aggressive disease.

The term “classifying the levels of expression of step (a) relative to levels of step (b)” as used herein means that the expression in a test sample for GOI's and the expression in a control sample for GOI's are compared, e.g. after normalization against a suitable normalization references and/or after calculating respective PCPI's.

According to the calculation of the PCPI's an individual may be considered to be eligible for a prostate cancer therapy when the gene expression levels are significantly different as compared with a control sample.

The term “stratifying an individual or cohort of individuals to prostate cancer therapy” as used herein means that an individual is identified as belonging to a group of similar individuals, whose optimal therapy form is a prostate cancer therapy, e.g. a therapy in accordance with the outcome of the calculation of the PCPI as described herein above.

An individual being considered to be eligible for prostate cancer therapy or being stratified to a prostate cancer therapy as described herein above may receive any suitable therapeutic treatment for this prostate cancer form known to the person skilled the art. In particular, the term “prostate cancer therapy” as used herein refers to any suitable prostate cancer therapy known to the person skilled in the art, and preferably includes surgical castration by removal of the testes as the main organ of male sex hormone production, chemical castration by e.g., hormone therapy such as suppression of generation of androgens or by inhibition of the androgen receptor activity, cytotoxic, chemotherapy, radiation therapy (External Beam Radiation Therapy, Brachytherapy), cryotherapy, focal therapies like HIFU ablation (High Frequency Ultrasound ablation), or thermal ablation or combinations thereof, preferably the combination of hormone therapy and radiation therapy.

Typically, an individual considered to be eligible for prostate cancer therapy may be deemed to be suffering from (aggressive) prostate cancer or be prone to develop such in the future, e.g. within the next 1 to 24 months. A correspondingly identified or stratified individual may be treated with an inhibitory pharmaceutical composition. In a further embodiment a correspondingly identified individual may be treated with an inhibitory pharmaceutical composition in combination with an additional cancer therapy. The term “additional cancer therapy” refers to any types of cancer therapy known to the person skilled in the art. Preferred are cancer therapy forms known for prostate cancer. The term includes, for example, all suitable forms of chemotherapy, radiation therapy, surgery, antibody therapies etc.

Alternatively, a correspondingly identified or stratified individual may also be treated solely with one or more cancer therapies such as a chemotherapy, radiation therapy, surgery, antibody therapies etc. Preferred are cancer therapies typically used for prostate cancer, more preferred cancer therapies used for aggressive prostate cancer.

In a further embodiment of the present invention the classification method for eligibility or for stratification as described herein above may also be used for monitoring the treatment of an individual, e.g. an individual being classified as suffering from an aggressive prostate cancer. The monitoring process may be carried out as expression determination over a prolonged period of time, e.g. during or after treatment sessions, for 1, 2, 3, 4, 5, 6, 7, 8, 9, 10 weeks, 1, 2, 3, 4, 5, 6, 7, 8, 9, 10 months, or 1, 2, 3 or more years. The determination steps may be carried out in suitable intervals, e.g. every week, 2 weeks, 3 weeks, every month, 2 months, 3 months, 6 months, 12 months etc. In a further embodiment of the present invention any treatment scheme as mentioned herein above may be adjusted, e.g. enforced or attenuated, or altered in any suitable manner in correspondence with the results of the monitoring process.

In a further embodiment of the present invention the method of identifying an individual for eligibility for aggressive prostate cancer therapy based on the expression of marker gene(s) or GOI's and the corresponding PCPI as described herein above may further be combined with one or more similar identification methods, based on the expression of one or more different biomarkers. Preferred is the determination of the level of prostate specific antigen (PSA) in blood. Thus, if the level of PSA in blood is encountered to be of a range of about 2 to 5 or more ng/ml, preferably of about 2.2 to 4.8 ng/ml or more, 2.4 to 4.4 ng/ml or more, 2.6 to 4.2 ng/ml ore more or 2.8 to 4.0 ng/ml or more, more preferably of about 2.5 to 4 ng/ml or more, an individual may be considered to be suffering from malignant prostate cancer, or be likely to develop malignant prostate cancer in the near future, i.e. within the next 1, 2, 3, 4, 5, 6, 12, 14, 48 months. The testing for expression of marker gene(s) or GOI's and determination of the PCPI may be carried out according to steps as defined herein above.

In a preferred embodiment of the present invention the diagnosing, detecting, monitoring or prognosticating as mentioned above is to be carried out on a sample obtained from an individual. The term “sample obtained from an individual” as used herein relates to any biological material obtained via suitable methods known to the person skilled in the art from an individual. The sample used in the context of the present invention should preferably be collected in a clinically acceptable manner, more preferably in a way that nucleic acids (in particular RNA) or proteins are preserved.

The biological samples may include body tissues and fluids, such as blood, sweat, and urine. Furthermore, the biological sample may contain a cell extract derived from or a cell population including an epithelial cell, preferably a cancerous epithelial cell or an epithelial cell derived from tissue suspected to be cancerous. Even more preferably the biological sample may contain a cell population derived from a glandular tissue, e.g. the sample may be derived from the prostate of a male individual. Additionally, cells may be purified from obtained body tissues and fluids if necessary, and then used as the biological sample.

Samples, in particular after initial processing, may be pooled. However, also non-pooled samples may be used.

In a specific embodiment of the present invention the content of a biological sample may also be submitted to an enrichment step. For instance, a sample may be contacted with ligands specific for the cell membrane or organelles of certain cell types, e.g. prostate cells, functionalized for example with magnetic particles. The material concentrated by the magnetic particles may subsequently be used for detection and analysis steps as described herein above or below.

In a specific embodiment of the invention, biopsy or resections samples may be obtained and/or used. Such samples may comprise cells or cell lysates.

Furthermore, cells, e.g. tumor cells, may be enriched via filtration processes of fluid or liquid samples, e.g. blood, urine, etc. Such filtration processes may also be combined with enrichment steps based on ligand specific interactions as described herein above.

In a particularly preferred embodiment of the present invention a sample may be a tissue sample, a biopsy sample, a urine sample, a urine sediment sample, a blood sample, a saliva sample, a semen sample, a sample comprising circulating tumor cells, or a sample containing prostate secreted exosomes. A blood sample, may, for example, be a serum sample or a plasma sample.

In the treatment of prostate cancer compounds or pharmaceutical compositions being active against cancer cells can be used. The pharmaceutical composition may comprise hormone-inhibitors, preferably anti-androgens or androgen antagonists like spironolactone, cyproterone acetate, flutamide, nilutamide, bicalutamide, ketoconazole, finasteride or dutasteride.

In a further specific embodiment the present invention envisages a method of monitoring the development of prostate cancer, which encompasses the determination of marker gene(s) or GOI's, preferably in combination with the determination of a reference gene as described herein above, over a certain period of time, i.e. after repeated determination steps, e.g. every 4 weeks, 6 weeks, two months, 4 months, 6 months, 8 months, 12 months, 1.5 years, 2 years, 2.5 years, 3 years, 4 years or any other suitable period of time etc. The method may provide data showing an increase or decrease of the level of marker gene(s) or GOI's in comparison to controls, e.g. non-cancerous controls, cancerous controls or to earlier data obtained from the same individual. With the help of suitable statistical methods known to the person skilled in the art the position within said curve may be determined. In dependence of the position within said curve, i.e. in an augmenting portion or a falling portion of said curve, the presence or future development prostate cancer may be diagnosed. Correspondingly, the use of pharmaceutical compositions is envisaged. Preferably, any such determination may be combined with the determination of secondary biomarkers, e.g. markers for prostate cancer, in particular PSA. In case of low PSA levels (up to 2.0 to 4.0 ng/ml) the GOI data may be analyses with respect to early prostate cancer, i.e. benign or hormone-dependent/hormone-sensitive prostate cancer. In case of higher PSA levels (higher than about 20 ng/ml, e.g. about 30, 40 or 50 ng/ml) the data may be analysed with respect to more advanced prostate cancer, e.g. hormone-resistant prostate cancer.

A “hormone-sensitive stage I to IV prostate cancer” as used herein denotes a prostate cancer which can be classified according to the TNM classification by the International Union Against Cancer (UICC) into stages I to IV.

A “hormone-sensitive recurrent prostate cancer” as used herein denotes a prostate cancer whose growth and progression is regulated and dependent on a male sex hormone. A preferred example of such a male sex hormone is an androgen.

A “hormone-sensitive metastatic prostate cancer” as used herein denotes prostate cancer whose growth and progression regulated and dependent on a male sex hormone. A preferred example of such a male sex hormone is androgen.

A “hormone-insensitive prostate cancer” as used herein denotes prostate cancer whose growth and progression is not regulated and independent on a male sex hormone. A preferred example of such a male sex hormone is androgen.

A further aspect of the invention relates to a computer program product, comprising computer readable code stored on a computer readable medium or downloadable from a communications network, which, when run on a computer, implement one or more steps or all the steps of any one of the methods as described herein.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified herein.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Unless defined otherwise all technical and scientific terms used herein have the same meaning as commonly understood to one of ordinary skill in the art to which this invention belongs.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

### Gene Selection to Build a Prostate Cancer Prognostic Index (PCPI)

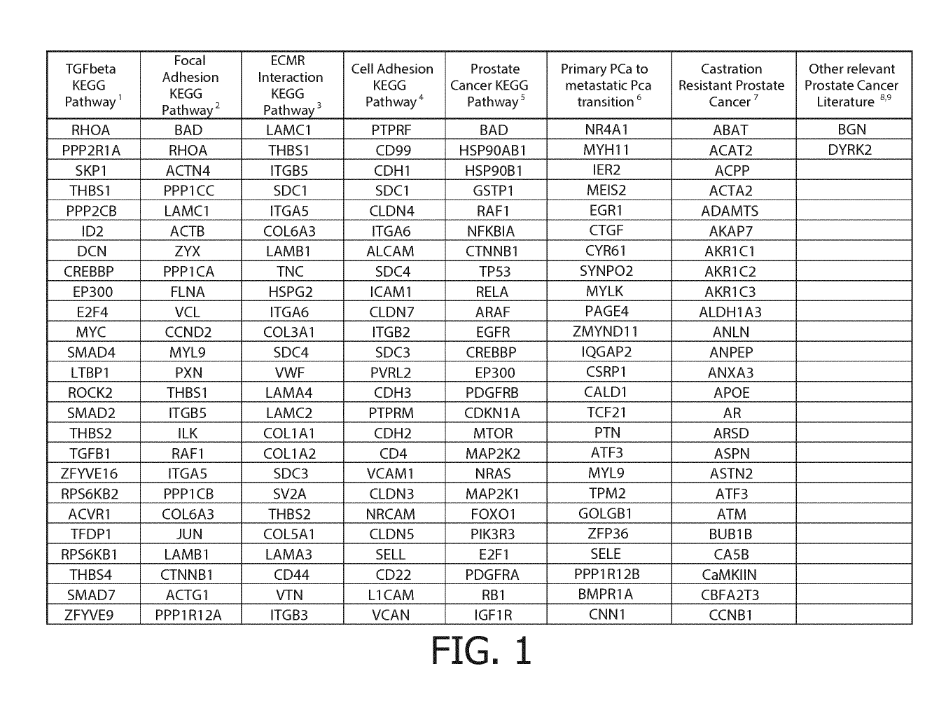

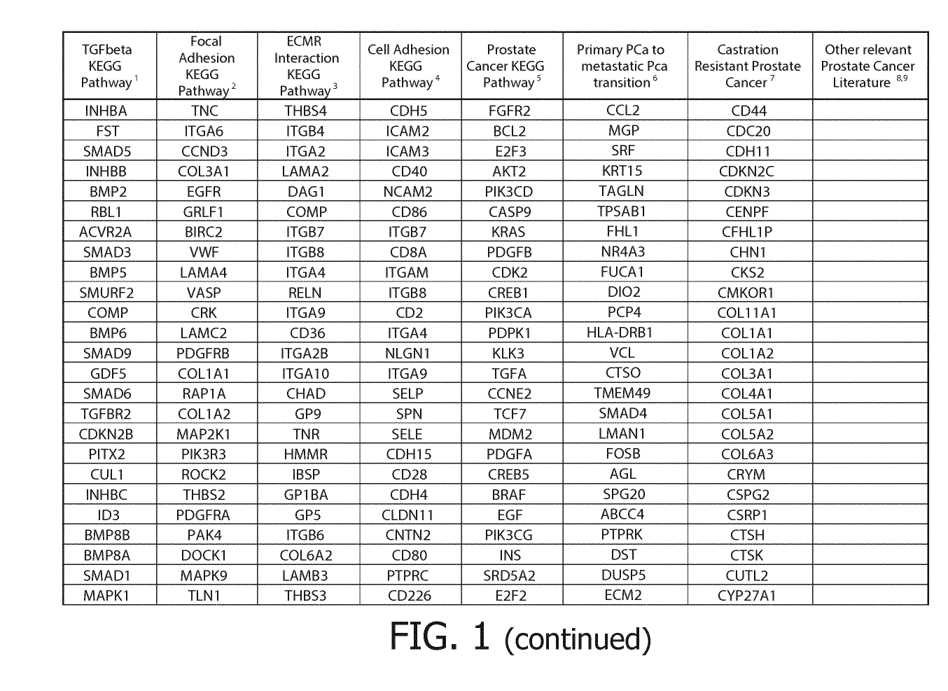

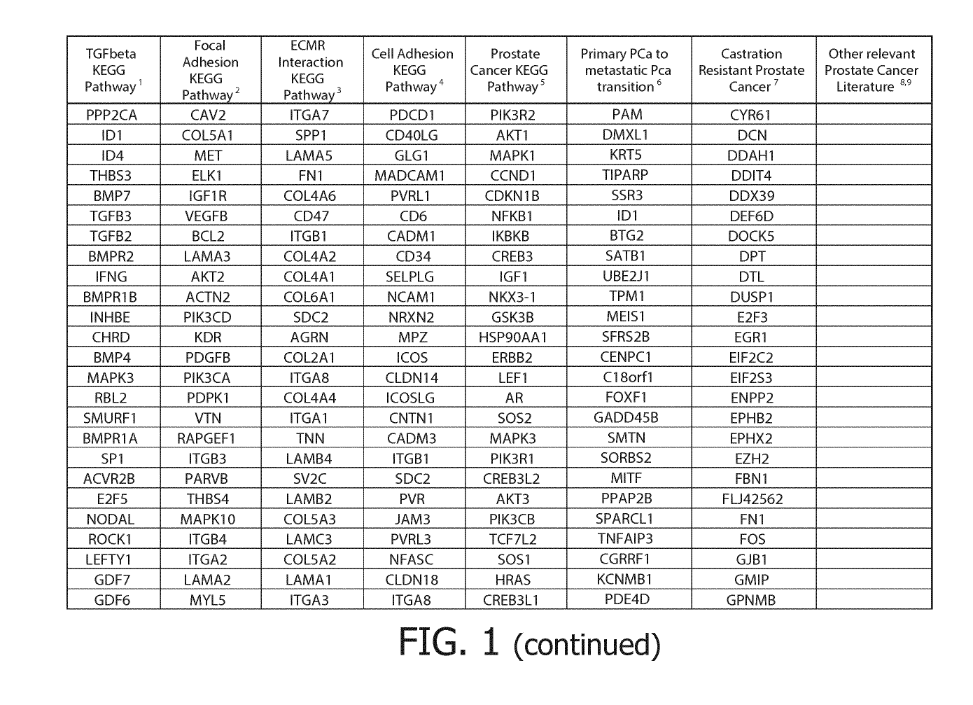

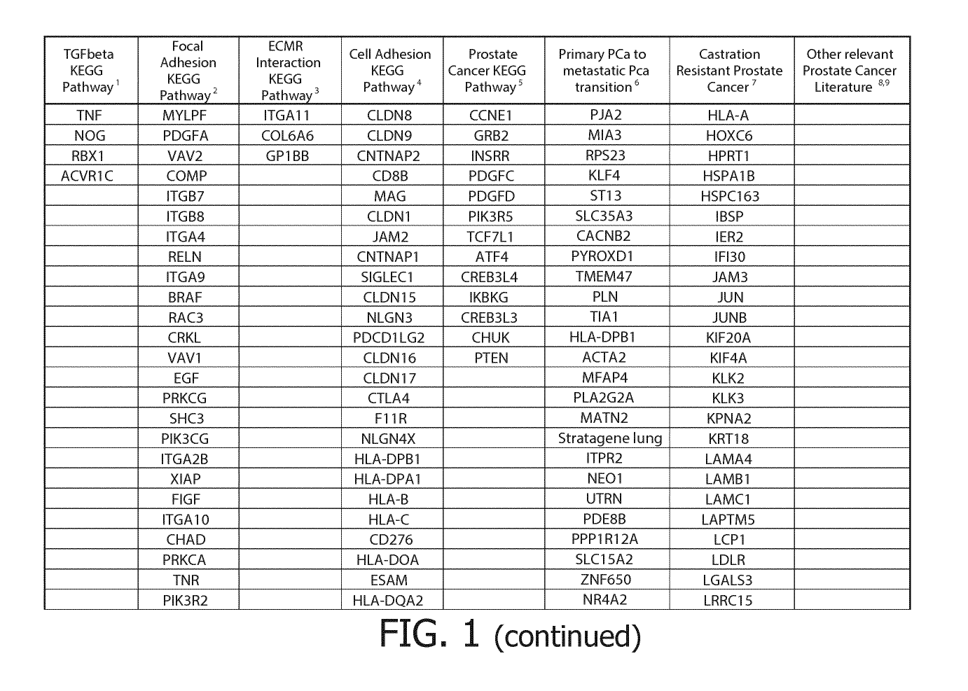

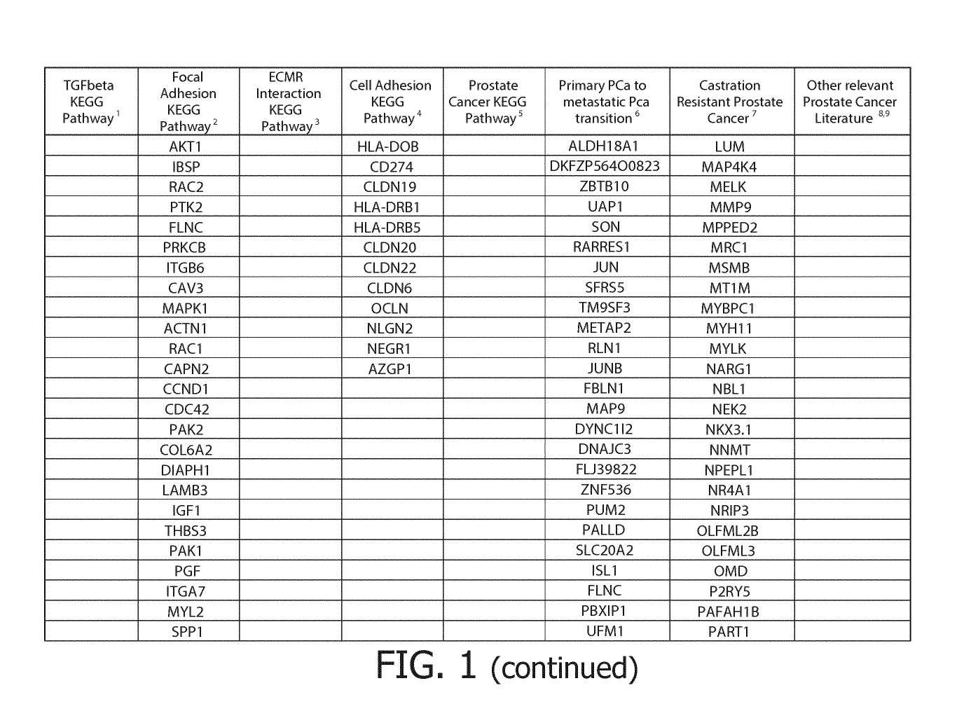

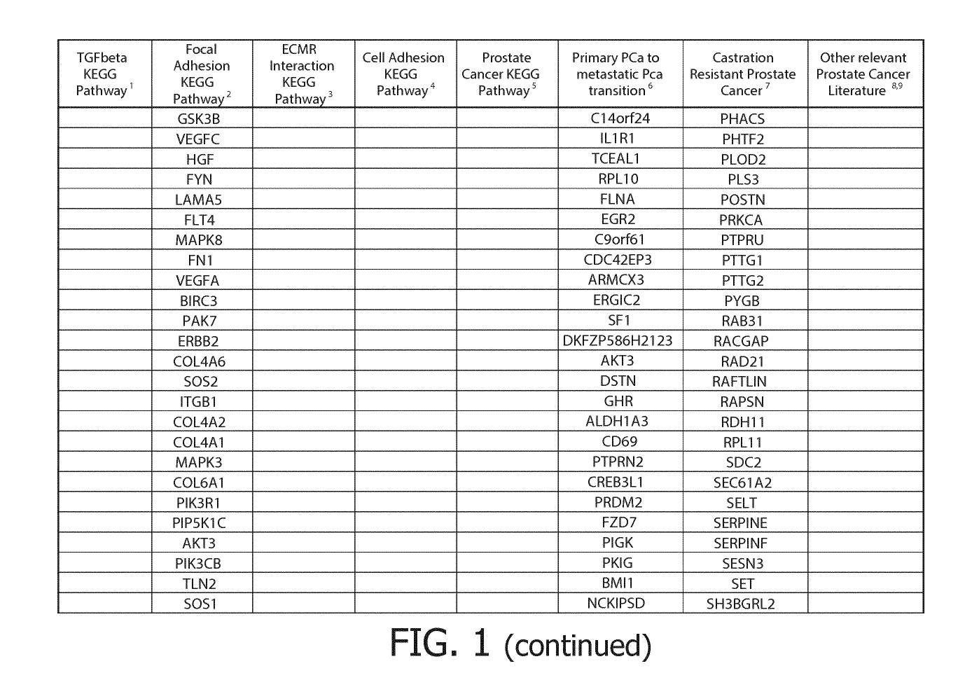

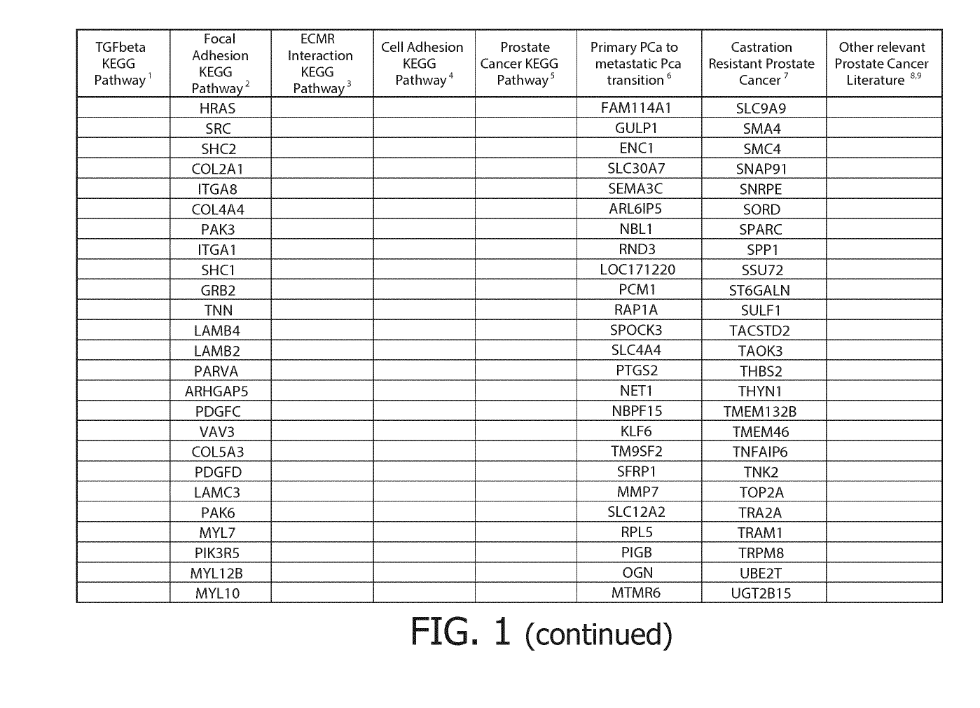

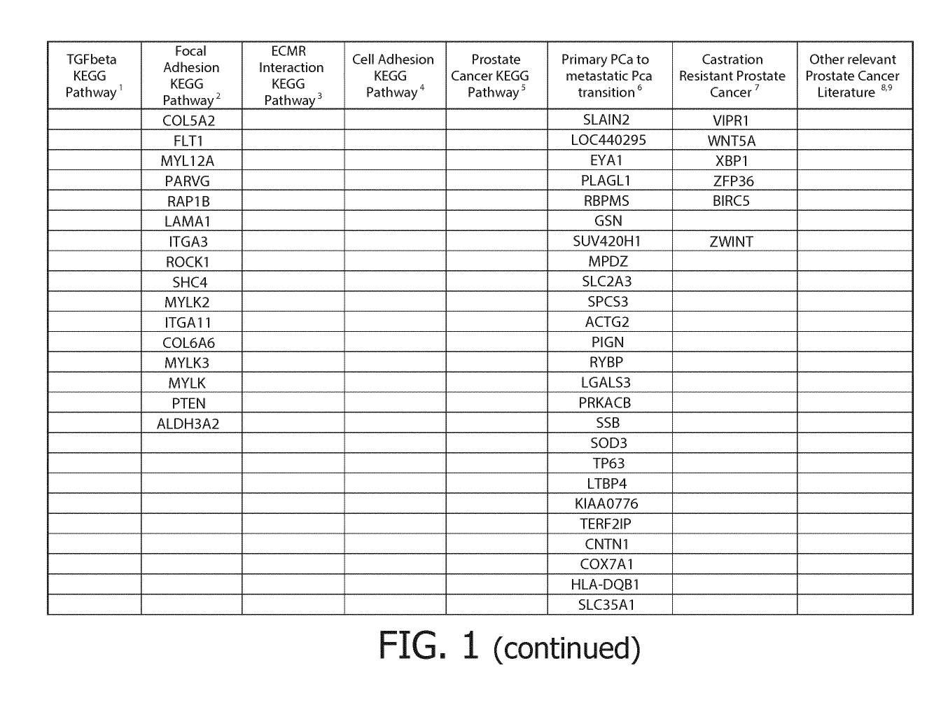

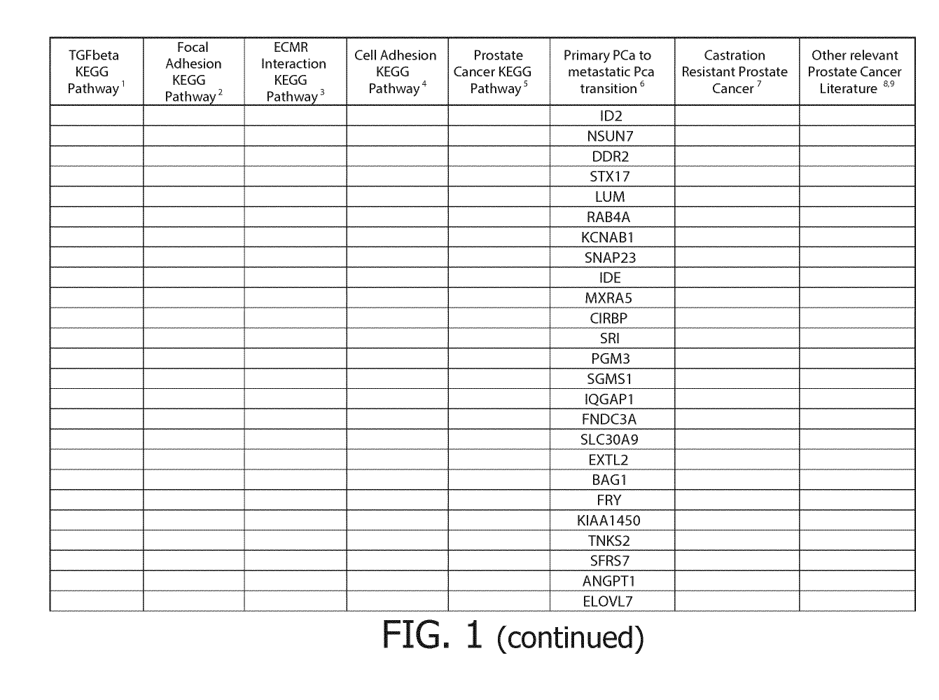

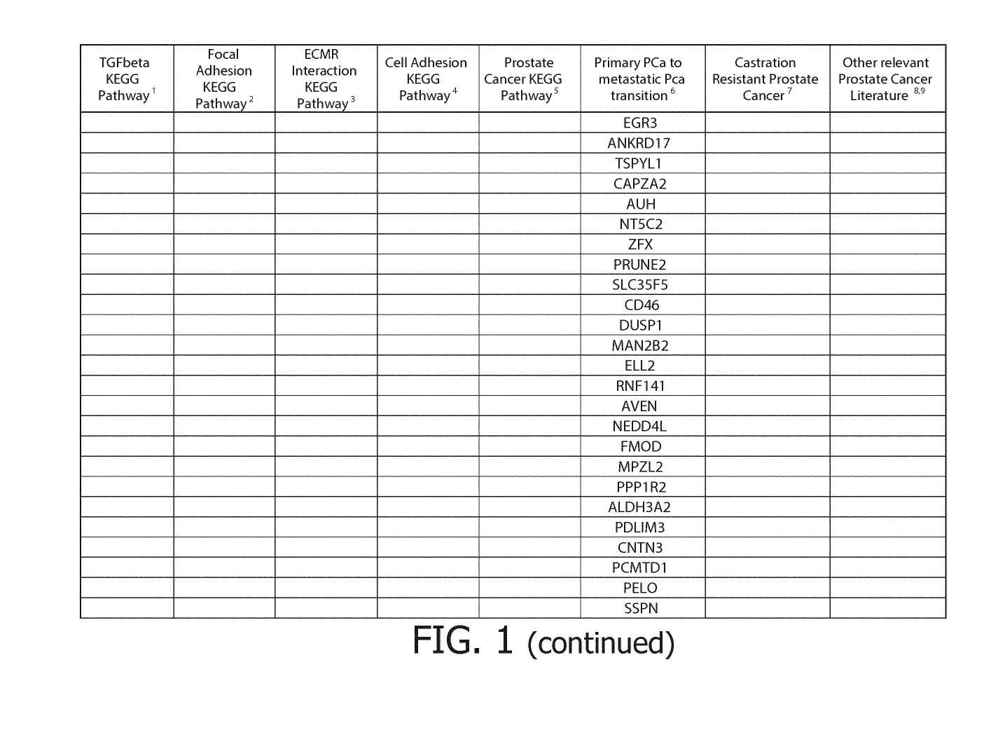

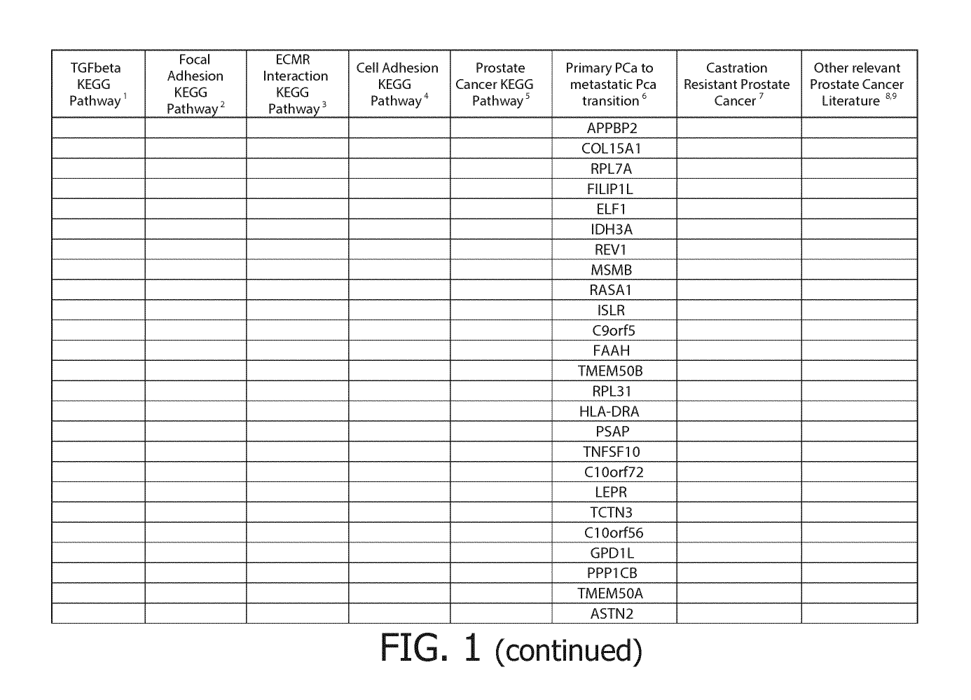

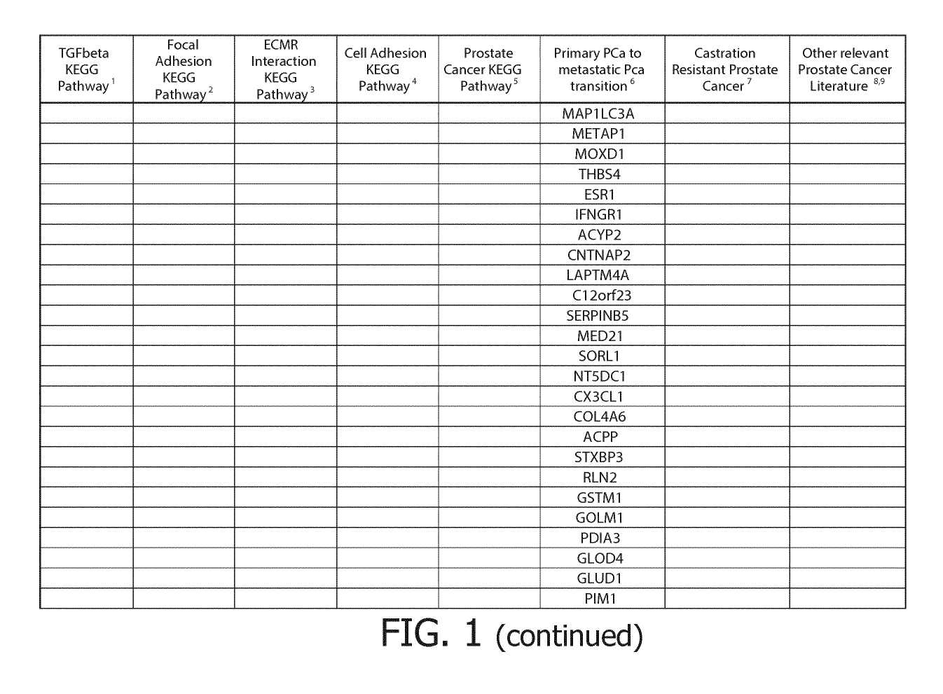

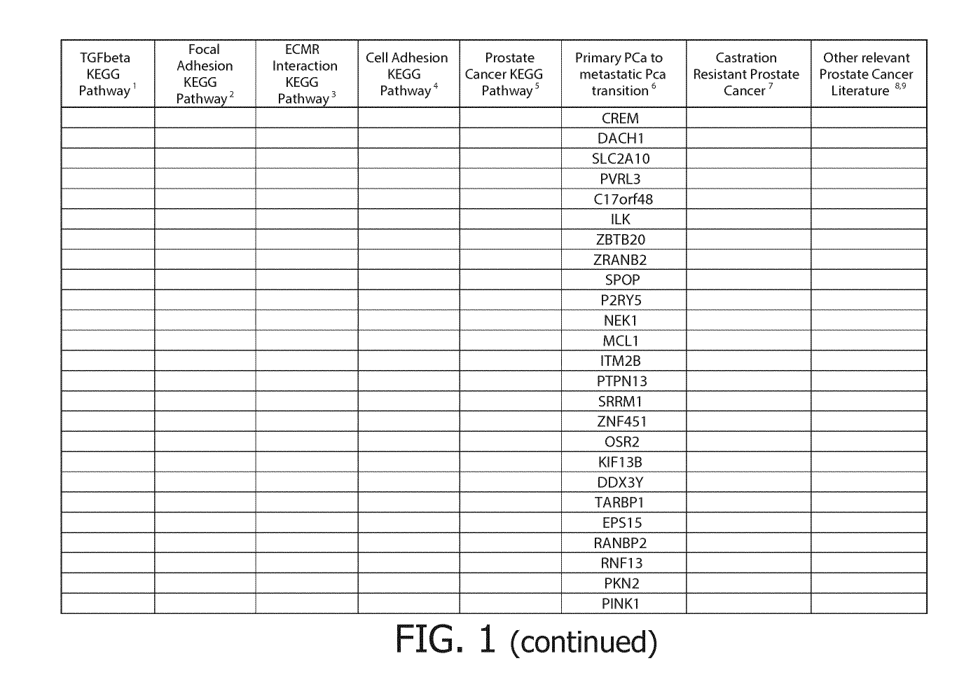

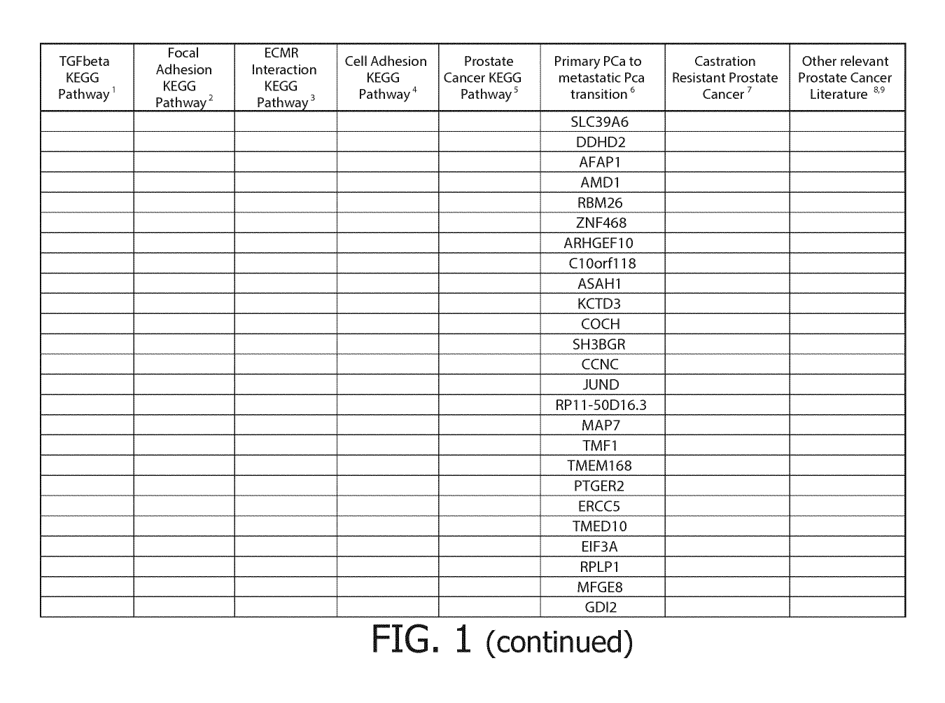

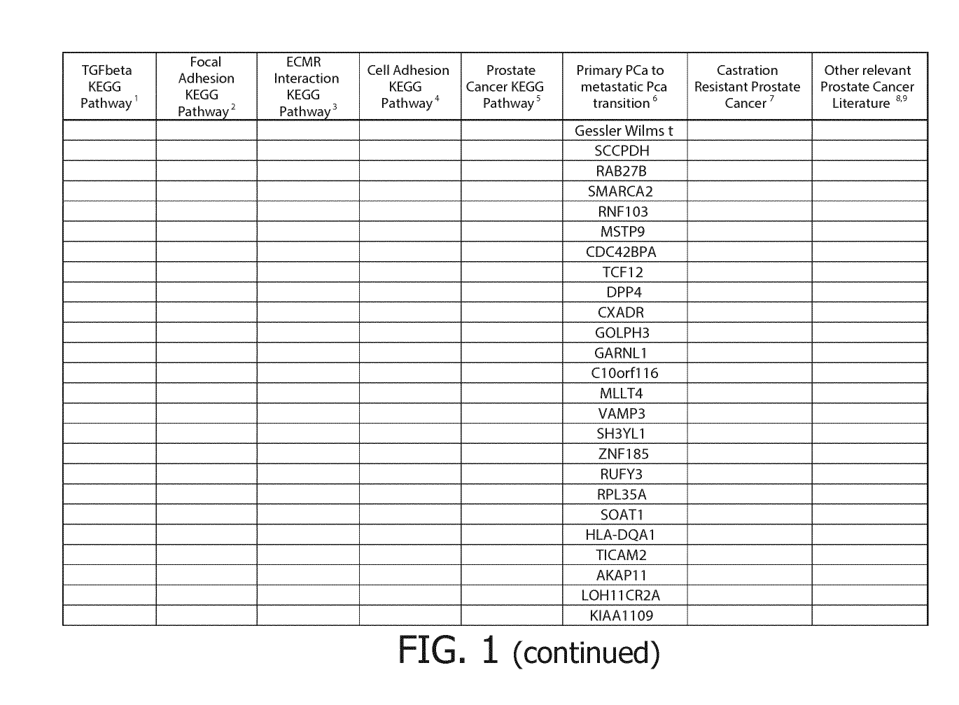

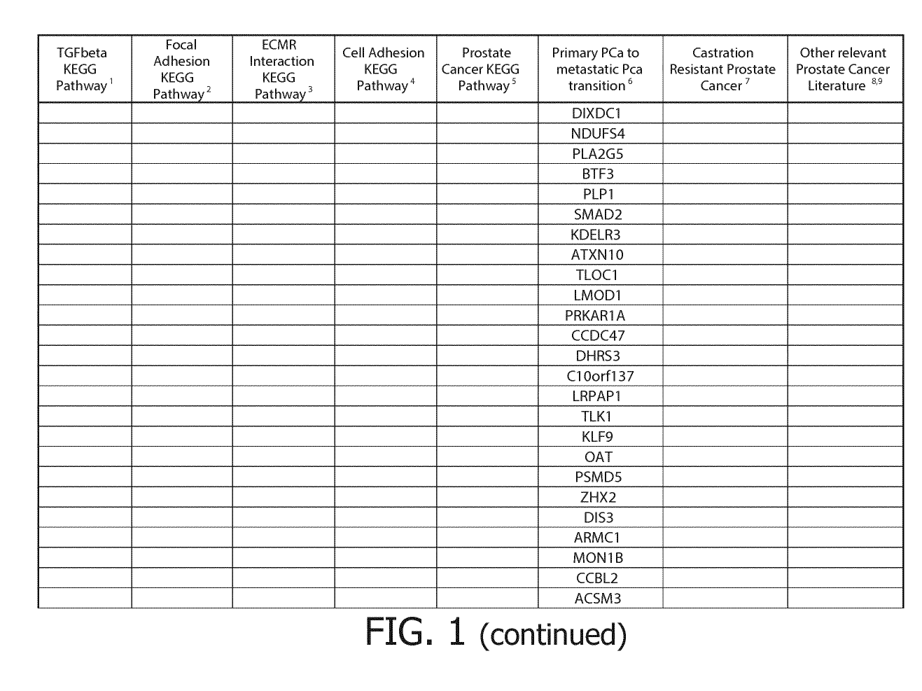

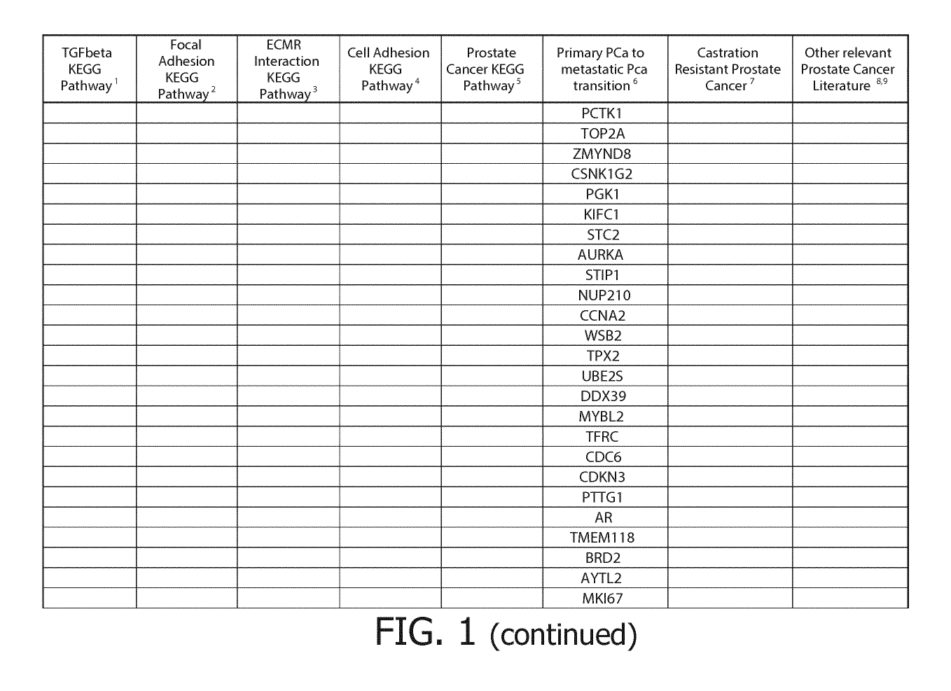

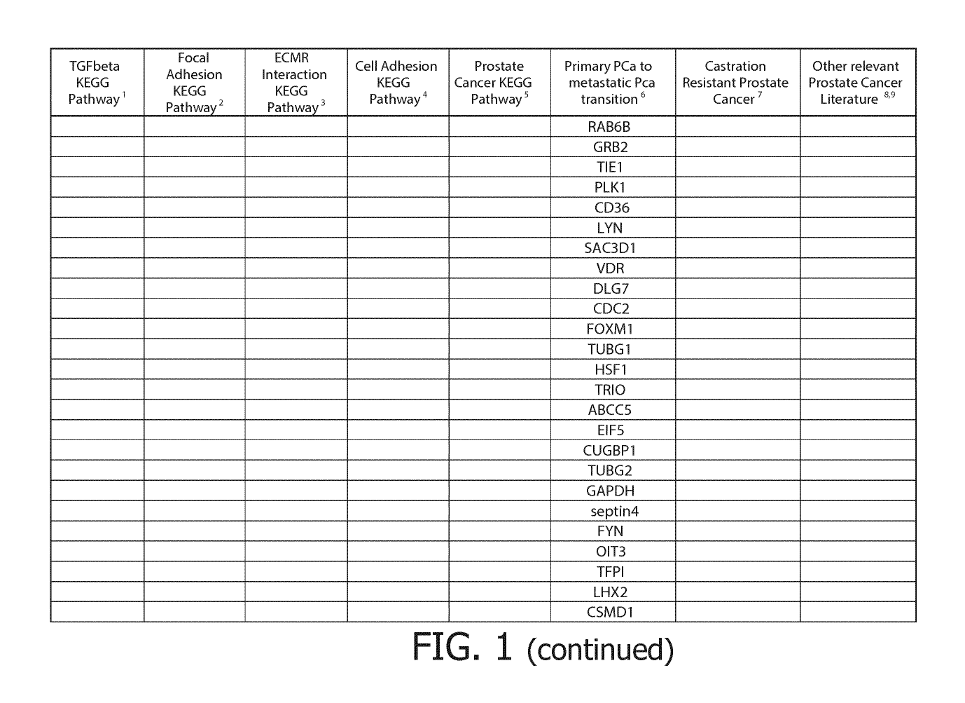

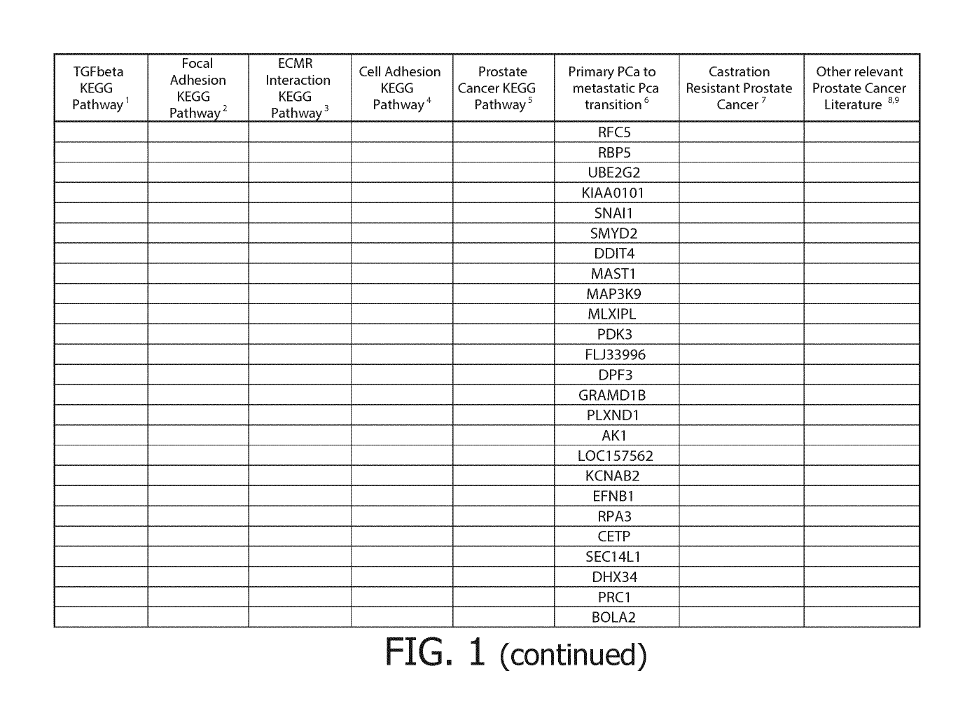

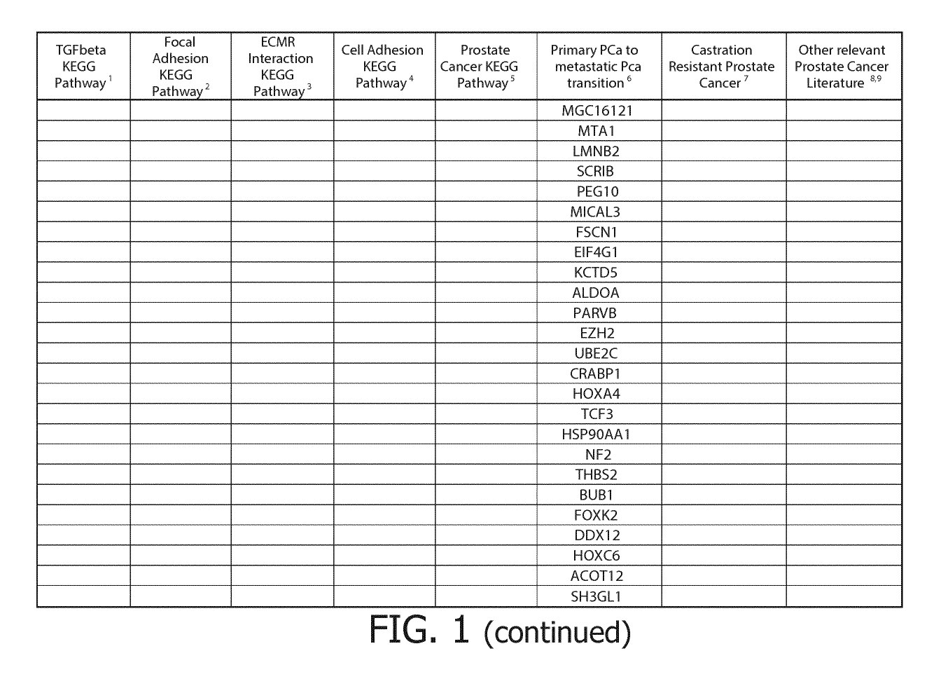

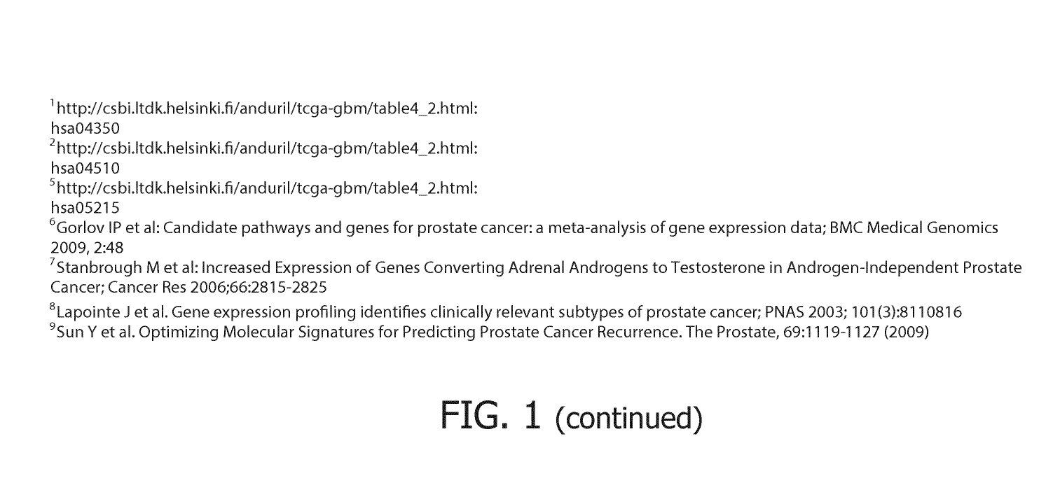

To select gene candidates to build the PCPI various molecular pathways (as outlined on http://csbi.ltdk.helsinkili/anduril/tcga-gbm/table4_2.html) were investigated for correlation of the putative prognostic gene marker PDE4D7 as described in WO2010131194 and WO2010131195. Those molecular pathways that showed significant correlation were selected for down-stream analysis (FIG. 1).

Furthermore, gene candidates related to metastatic and/or hormone-refractory prostate cancer were selected from the following literature
Gorlov IP et al. Candidate pathways and genes for prostate cancer: a meta-analysis of gene expression data; BMC Medical Genomics 2009, 2:48
Stanbrough M et al: Increased Expression of Genes Converting Adrenal Androgens to Testosterone in Androgen-Independent Prostate Cancer; Cancer Res 2006;66:2815-2825
Tamura K et al. Molecular Features of Hormone-Refractory Prostate Cancer Cells by Genome-Wide Gene Expression Profiles; Cancer Res 2007; 67(11):5117-25
Lapointe J et al. Gene expression profiling identifies clinically relevant subtypes of prostate cancer; PNAS 2003; 101(3):8110816
Sun Y et al. Optimizing Molecular Signatures for Predicting Prostate Cancer Recurrence. The Prostate, 69:1119-1127 (2009)

In total 967 genes (cf. FIG. 1) reported to be potentially relevant to prostate cancer progression were selected for further analysis.

To build a 19-gene prognostic signature, i.e. a set of marker genes of interest, the following data sets were used:
Taylor B S et al. Integrative Genomic Profiling of Human Prostate Cancer. Cancer Cell 18, 11-22, 2010 (GEO data set ID: GSE21032)
Boormans J L et al. Identification of TDRD1 as a direct target gene of ERG in primary prostate cancer. Int J Cancer 2013 Jul. 15; 133(2):335-45 (GEO data set ID: GSE41408)
Sun Y et al. Optimizing Molecular Signatures for Predicting Prostate Cancer Recurrence. The Prostate, 69:1119-1127 (2009) (GEO data set ID: GSE25136)
Sboner A et al. Molecular sampling of prostate cancer: a dilemma for predicting disease progression. BMC Medical Genomics 2010, 3:8 (GEO data set ID: GSE16560)
Nagakawa T et al. A Tissue Biomarker Panel Predicting Systemic Progression after PSA Recurrence Post-Definitive Prostate Cancer Therapy. PLoS ONE 3(5), e2318 (2010) (GEO data set ID: GSE10645)

To further test and validate the built Prostate Cancer Prognostic Index (PCPI) the following data set was used:

Erho N et al. Discovery and Validation of a Prostate Cancer Genomic Classifier that Predicts Early Metastasis Following Radical Prostatectomy. PLoS One 8(6), e66855 (2013) (GEO data set ID: GSE46691)

Processing of GSE Gene Expression Data

Respective CEL files were downloaded from GEO (Gene Expression Omnibus): http://www.ncbi.nlm.nih.gov/geo/. The CEL file data were uploaded into Expression Console (Affymetrix Inc; Build 1.3.1.187) and were pre-processed using the appropriate probe set annotation files provided by Affymetrix Inc. where appropriate, i.e., data sets run on an Affymetrix Inc platform: GSE21032, GSE41408, GSE25136. For data sets run on the DASL platform (i.e., GSE16560, GSE10645) the data series matrix files were used as provided by GEO for downstream data analysis.

Reference genes are supposed to have a stable expression independently of the sample being processed and therefore can be used as an internal standard to normalize the output Cq values of a PCR analysis so that the results are comparable independently of the amount of input sample used. Though such genes are supposed to be stable, there is always some variability in their expression, and this stability can also depend on the tissue being analyzed. For such reason it is recommended to use more than one reference gene in the normalization of PCR Cq values and to use a set of genes that presents low variability in the specific type of tissue/sample being analyzed (C. L. Andersen, J. L. Jensen, and T. F. Ørntoft, “Normalization of Real-Time Quantitative Reverse Transcription-PCR Data: A Model-Based Variance Estimation Approach to Identify Genes Suited for Normalization, Applied to Bladder and Colon Cancer Data Sets,” Cancer Res., vol. 64, no. 15, pp. 5245-5250, August 2004; J. Vandesompele, K. De Preter, F. Pattyn, B. Poppe, N. Van Roy, A. De Paepe, and F. Speleman, “Accurate normalization of real-time quantitative RT-PCR data by geometric averaging of multiple internal control genes,” Genome Biol., vol. 3, no. 7, p. RESEARCH0034, June 2002).

An initial selection of 9 reference genes to be used in this study was performed. Final selection of reference gene set used for normalization was done by running the with the Biogazelle Software tool (qBase+ with GeNorm analysis J. Hellemans, G. Mortier, A. D. Paepe, F. Speleman, and J. Vandesompele, “qBase relative quantification framework and software for management and automated analysis of real-time quantitative PCR data,” Genome Biol., vol. 8, no. 2, p. R19, February 2007.) on all the analyzed clinical samples.

The output Cq values of a PCR assay are intrinsically logarithmic (base 2) and inversely proportional to the amount of mRNA present in the sample. We use the following formula to normalize the raw Cq values:

N(Cq_{gene of interest})=Mean(Cq_{ref gene})−(Cq_{gene of interest})

Where N(Cq_{gene of interest}) is normalized gene expression value for selected genes of interest; where Mean(Cq_{ref}gene) is the arithmetic mean of the PCR Cq values of the selected combination of reference genes; where (Cq_{gene of interest}) is the PCR Cq value of the gene of interest.

In case other technologies than qRT-PCR are used to measure the expression of the reference genes or the genes of interest the PCR Cq value will be replaced by a normalized measurement of the respective technology (e.g., a RMA (Robust Multi-array Average) normalized gene expression value for DNA microarrays, or a FPKM (Fragements Per Kilobase of Exon Per Million Fragments Mapped) normalized gene expression value for RNA sequencing).

Calculation of Prostate Cancer Progression Index (PCPI)

Selected Genes of Interest (GOI) were grouped into those down-regulated between patient groups with and without progression after primary treatment compared to those up-regulated between patient groups with and without progression after primary treatment. An average gene expression value for the down-regulated genes was calculated (GEV_av_GOI_down) as well as an average gene expression value for the up-regulated genes (GEV_av_GOI_up) from the normalized gene expression data per each analyzed patient sample. The Prostate Cancer Progression Index PCPI per patient sample was determined as:

PCPI=(GEV_av_GOI_up)−(GEV_av_GOI_down)

The calculated PCPI was used for any further statistical data analysis.

PCPI_17 Computation (Based on Quantitative Real-Time PCR Data)

The PCPI was computed using the following formula:

PCPI=Mean(N(Cq_{PCPI genes up}))−Mean(N(Cq_{PCPI genes down}))

where

Mean(N(Cq_{PCPI genes up})) is the arithmetic mean of the normalized gene expression values (see above) for the genes: BGN, BIRC5, COL1A1, COL3A1, COL5A2, DYRK2, INHBA, THBS2, and VCAN

and where

Mean(N(Cq_{PCPI genes down})), is the arithmetic mean of the normalized gene expression values (see above) for the genes: AZGP1, FBLN1, ILK, KRT15, MEIS2, MYBPC1, PAGE4, and SRD5A2.

PCPI_18 Computation (Based on Quantitative Real-Time PCR Data)

The PCPI was computed using the following formula:

PCPI=Mean(N(Cq_{PCPI genes up}))−Mean(N(Cq_{PCPI genes down}))

where

Mean(N(Cq_{PCPI genes up})) is the arithmetic mean of the normalized gene expression values (see above) for the genes: BGN, BIRC5, COL1A1, COL3A1, COL5A2, DYRK2, INHBA, THBS2, and VCAN

and where

Mean(N(Cq_{PCPI genes down})), is the arithmetic mean of the normalized gene expression values (see above) for the genes: PDE4D, AZGP1, FBLN1, ILK, KRT15, MEIS2, MYBPC1, PAGE4, and SRD5A2

PCPI_19 Computation (Based on Quantitative Real-Time PCR Data)

The PCPI was computed using the following formula:

PCPI=Mean(N(Cq_{PCPI genes up}))−Mean(N(Cq_{PCPI genes down}))

where

Mean(N(Cq_{PCPI genes up})) is the arithmetic mean of the normalized gene expression values (see above) for the genes: BGN, BIRC5, COL1A1, COL3A1, COL5A2, DYRK2, INHBA, THBS2, and VCAN

and where

Mean(N(Cq_{PCPI genes down})), is the arithmetic mean of the normalized gene expression values (see above) for the genes: PDE4D5, PDE4D7, AZGP1, FBLN1, ILK, KRT15, MEIS2, MYBPC1, PAGE4, and SRD5A2.

Correlation Analysis of the Prostate Cancer Progression Index (PCPI) vs. Clinical Parameters to Patient Outcome

All statistical analysis (e.g., logistic regression analysis, ROC analysis, COX regression analysis, etc.) of the PCPI and its correlation to patient outcome in comparison to clinical parameters like PSA, pGleason, pT Stage was performed with XLSTAT Version 2014.1.03

Results

The 967 selected gene candidates were investigated for differential expression in the above gene expression data sets between prostate cancer patients that did not show progression of their disease after primary treatment vs. those patient groups that demonstrated disease progression to either of the following clinical endpoints: biochemical recurrence, clinical recurrence by detection of local or distant metastases, prostate cancer specific death.

Table 2 (cf. FIG. 2) provides an overview of 57 selected genes that were found to be significantly (p<0.05) differentially expressed and were regulated between patient groups in the same direction (i.e., either up- or down-regulated) between above mentioned patient groups at least in three out of the five tested data sets. Subsequently, genes were ranked into three categories according to the following criteria:

Rank_3: all genes that were observed in max 3 data sets to be differentially expressed with a significant p-value (p<0.05) in the same direction or genes with non-unique annotation

Rank_2: all genes that were observed in 4 or 5 data sets to be differentially expressed with a significant p-value (p<0.05) in the same direction

Rank_1: all genes that were observed in 4 or 5 data sets to be differentially expressed with a significant p-value (p<0.05) in the same direction but limited to 9 genes up-regulated as well as 10 genes down-regulated

A preferred PCPI signature comprises or consists of the following 19 genes: PDE4D5, PDE4D7, AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2.

As not all data sets provide information on transcript level of PDE4D isoforms (i.e., PDE4D5 and PDE4D7) the demonstrated results are based on the measurement of the PDE4D gene as the expression of PDE4D is very well correlated to the expression of PDE4D7 (normalized to the expression of PDE4D5) in data sets GSE21032 and GSE41408); consequently, the determined PCPI_18 comprised the following 18 genes: PDE4D, AZGP1, FBLN1, ILK, KRT15, MEIS2, MYBPC1, PAGE4, SRD5A2, COL1A1, COL3A1, INHBA, THBS2, VCAN, BGN, BIRC5, DYRK2.

For such data sets where the PDE4D gene was not measured (i.e., GSE16560, GSE10645) the determined PCPI_17 comprised the following 17 genes: AZGP1, FBLN1, ILK, KRT15, MEIS2, MYBPC1, PAGE4, SRD5A2, COL1A1, COL3A1, INHBA, THBS2, VCAN, BGN, BIRC5, DYRK2.

Correlation of the PCPI to Clinical Outcome Data of Prostate Cancer Patients after Primary Treatment

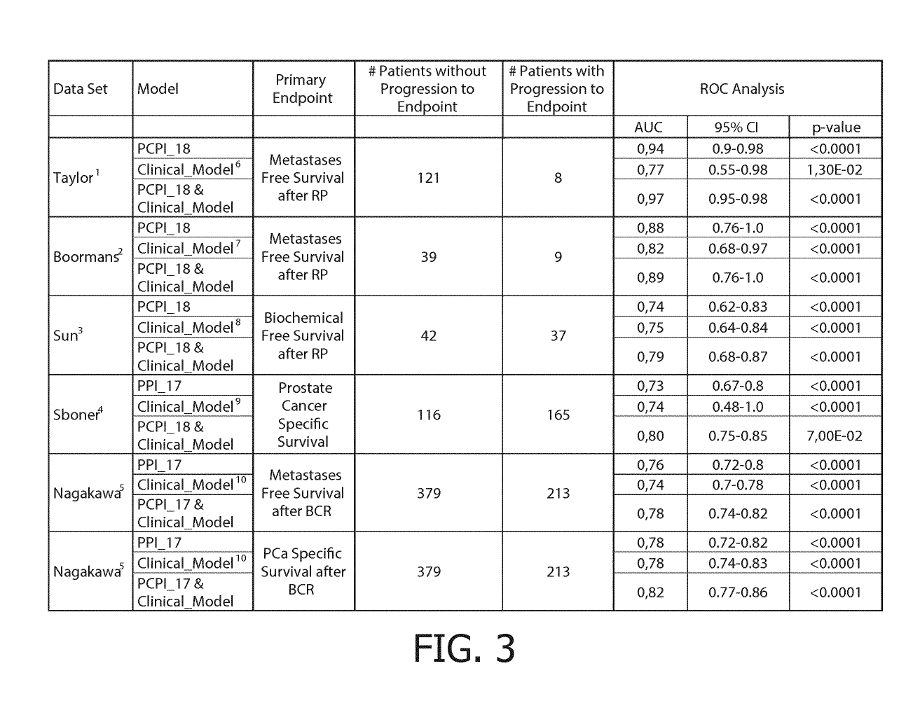

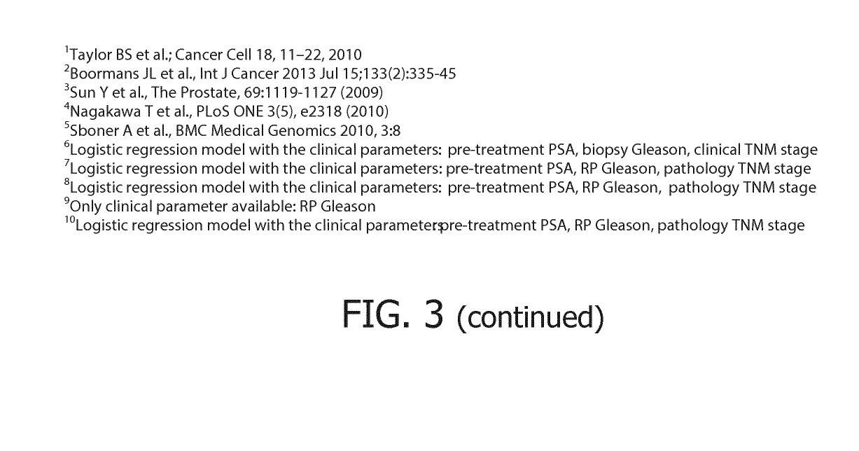

Table 3 (cf. FIG. 3) shows an overview on the performance of PCPI_18 or PCPI_17 to predict the primary endpoint of development of distant metastases within 10-15 years after primary treatment (prostate surgery in general) for the data sets GSE21032, GSE41408, GSE25136, GSE16560, GSE10645. For patients investigated in data set GSE16560 the cancers of the prostate were detected during a TURP (transurethral resection) and patients were subsequently managed conservatively (i.e., without removal of the prostate). For patients investigated in data set GSE10645 the monitoring of patients for the endpoint did only start after biochemical recurrence). These data sets were used to build the PCPI signature (training data). The number of patients per group (no progression vs. progression to metastases) is shown as well as the AUC (Area Under the Curve) derived from a ROC analysis. The AUC indicates the relative power of the PCPI in the different data sets to predict the primary endpoint within the cohort).

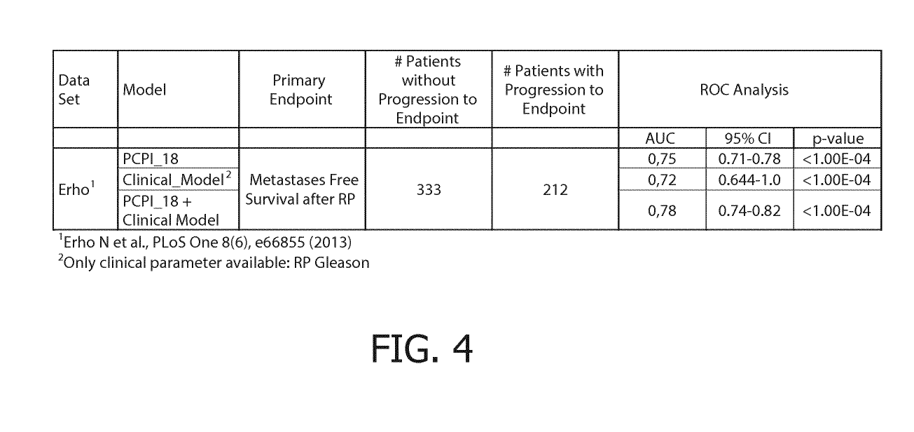

Table 4 (cf. FIG. 4) shows the performance of PCPI_18 to predict the primary endpoint of development of distant metastases within 10-15 years after primary treatment for the data set GSE46691; this data set was not used to train the PCPI and is used as an independent verification data set (validation data). For patients investigated in data set GSE10645 the monitoring of patients for the endpoint did only start after biochemical recurrence). The number of patients per group (no progression vs. progression to metastases) is shown as well as the AUC (Area Under the Curve) derived from a ROC analysis. The AUC indicates the relative power of the PCPI in the different data sets to predict the primary endpoint within the cohort.

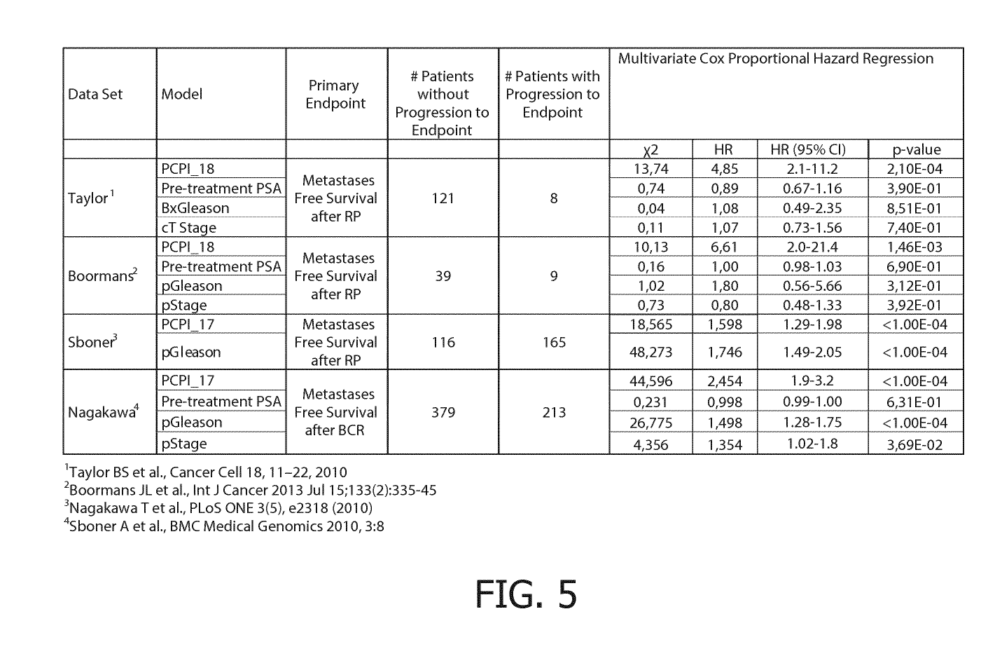

Table 5 (cf. FIG. 5) shows the performance of PCPI_18 or PCPI_17 to predict the primary endpoint of development of distant metastases within 10-15 years after primary treatment (prostate surgery in general) for the data sets GSE21032, GSE41408, GSE16560, GSE10645 in a multivariate COX regression analysis compared to standard clinical parameters like pre-treatment PSA, biopsy or pathology Gleason score, or clinical/pathology disease stage. The Chi Square as well as the Hazard Ratio for the PCPI and the individual clinical parameters is shown for each individual data set.

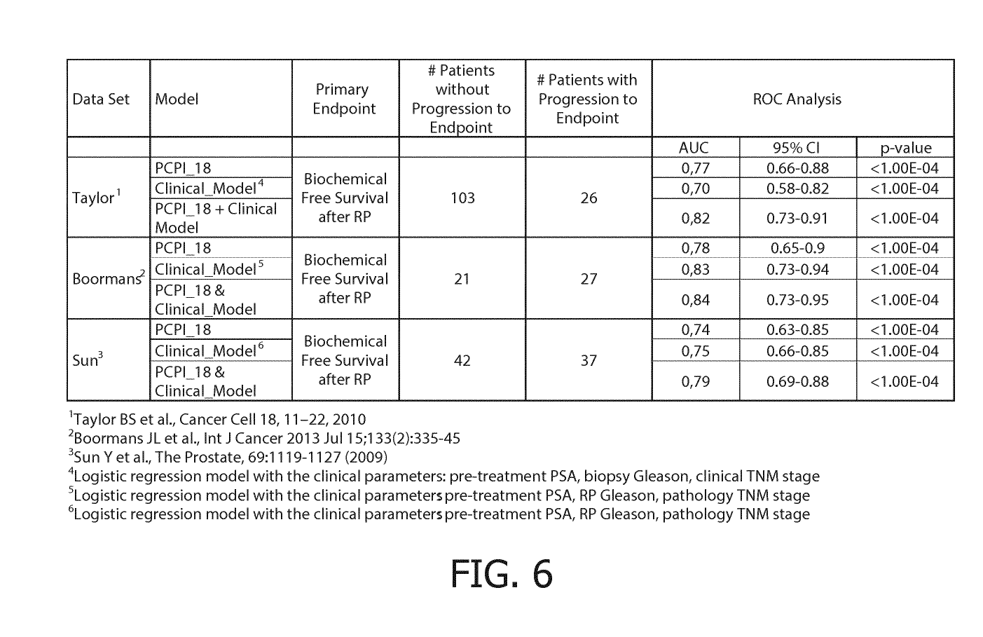

Table 6 (cf. FIG. 6) shows an overview on the performance of PCPI_18 to predict the primary endpoint of development of biochemical disease recurrence (BCR) within 10-15 years after primary treatment (prostate surgery in general) for the data sets GSE21032, GSE41408, GSE25136. The number of patients per group (no progression vs. progression to BCR) is shown as well as the AUC (Area Under the Curve) derived from a ROC analysis. The AUC indicates the relative power of the PCPI in the different data sets to predict the primary endpoint within the cohort).

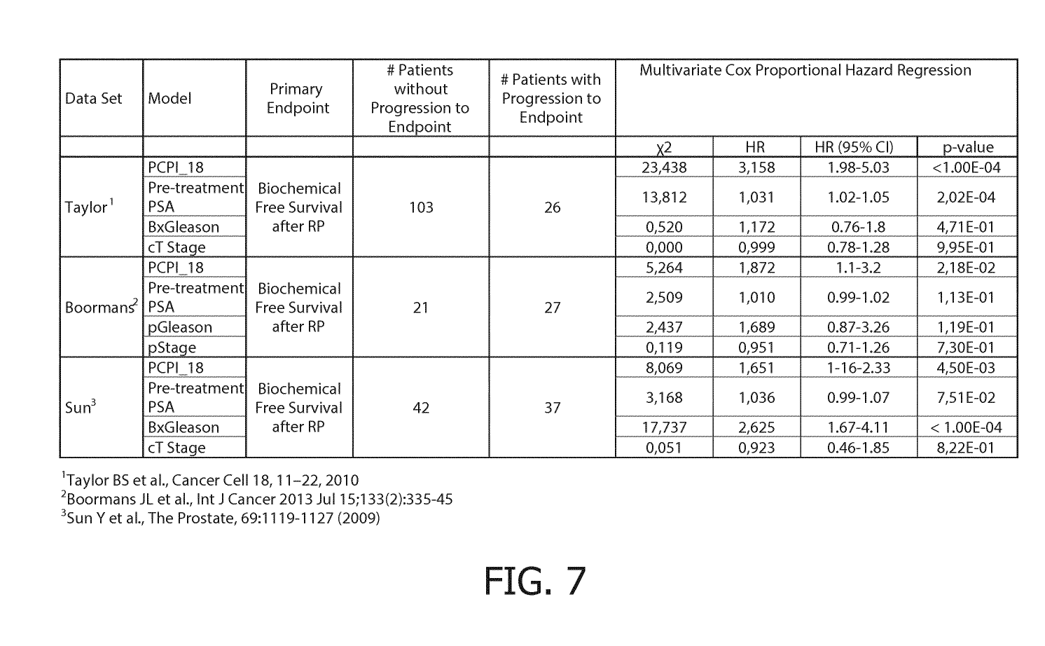

Table 7 (cf. FIG. 7) shows the performance of PCPI_18 to predict the primary endpoint of development of biochemical disease recurrence (BCR) within 10-15 years after primary treatment (prostate surgery in general) for the data sets GSE21032, GSE41408, GSE25136 in a multivariate COX regression analysis compared to standard clinical parameters like pre-treatment PSA, biopsy or pathology Gleason score, or clinical/pathology disease stage. The Chi Square as well as the Hazard Ratio for the PCPI and the individual clinical parameters is shown for each individual data set.

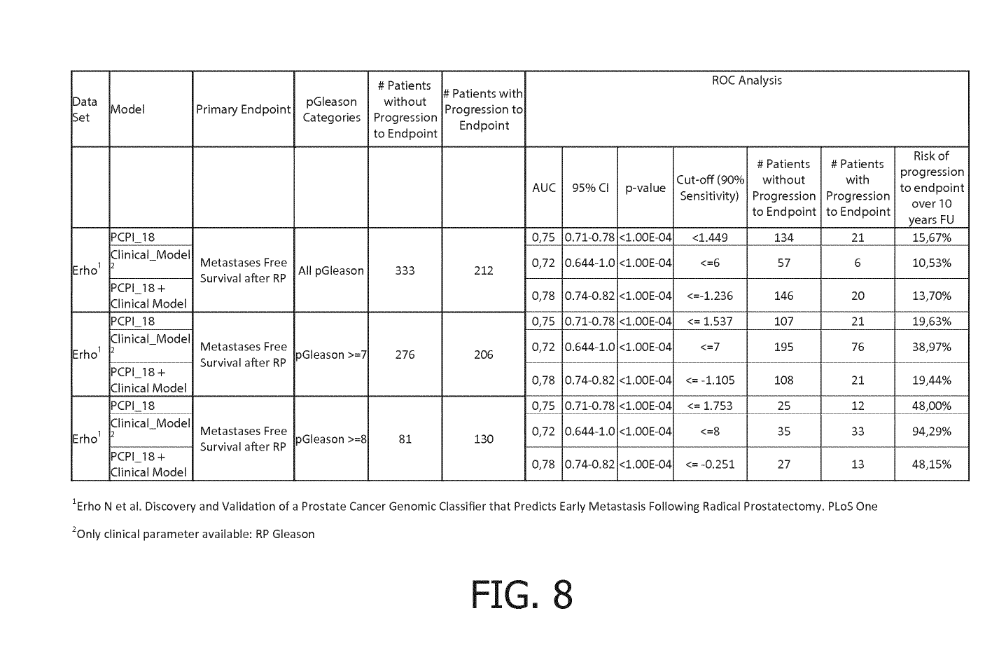

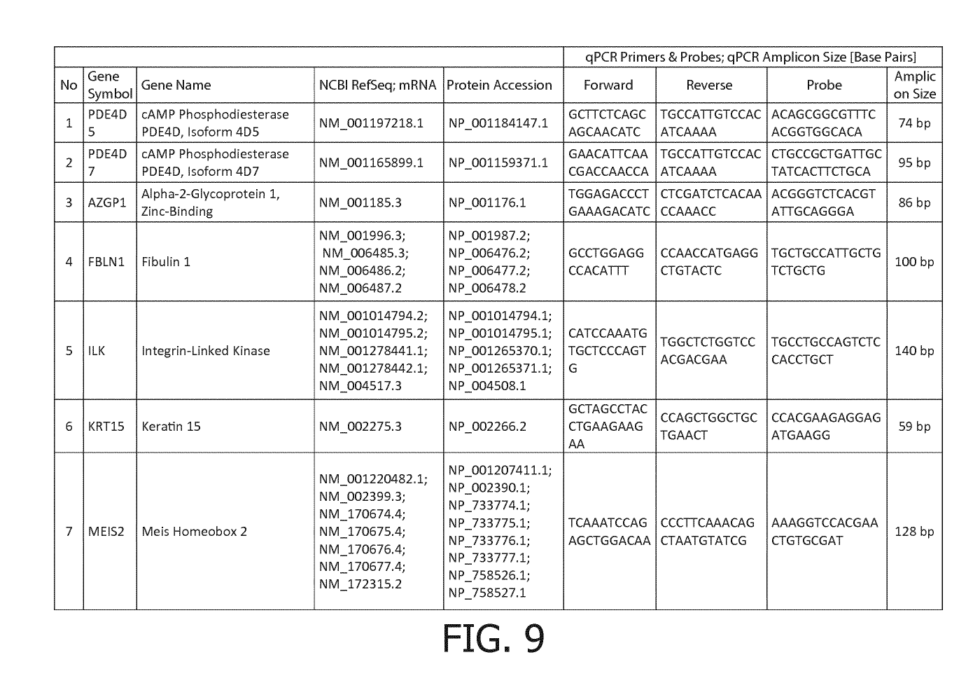

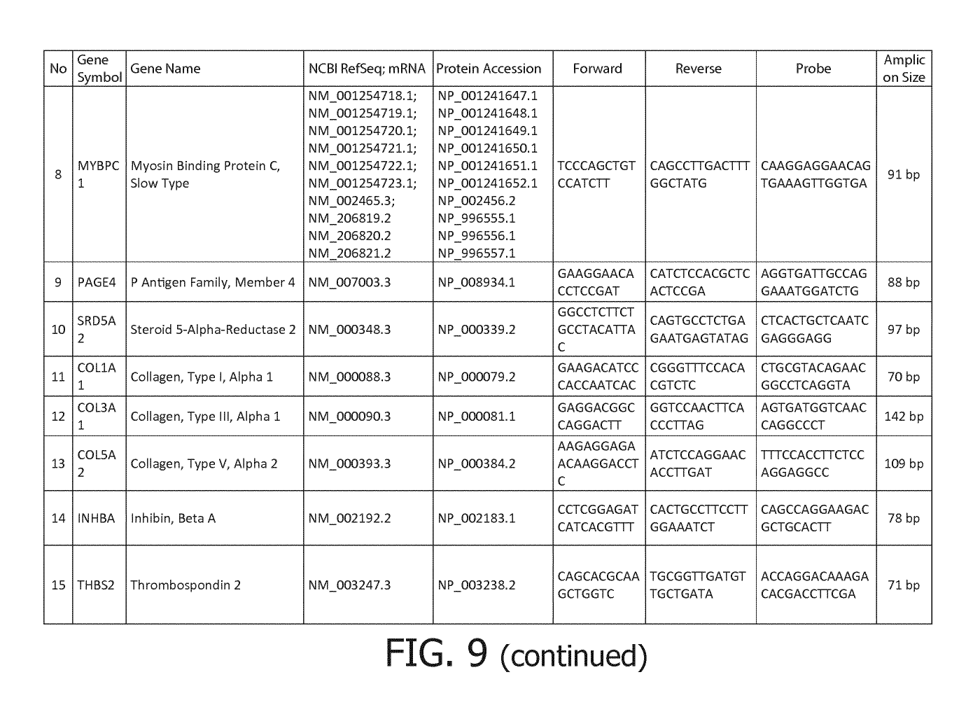

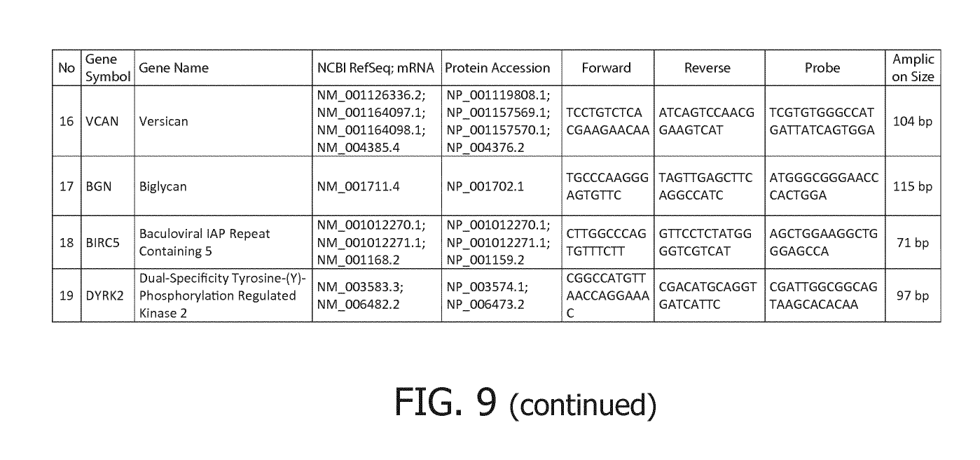

Table 8 (cf. FIG. 8) shows a ROC curve cut-off analysis for PCPI_18 to support clinical decision making towards patient stratification for either active surveillance (AS) or active treatment (e.g., prostatectomy, radiotherapy, hormone therapy). The cut-off as shown in the table was selected from the ROC curve such that >=90% of men with aggressive disease were correctly classified (corresponding to 90% sensitivity). This cut-off allows the stratification of 134 with a very low-risk profile towards e.g. active surveillance instead of e.g. surgery. The progression-free survival in this cohort to the endpoint of metastatic disease over approx. 10 years follow-up is close to 85%. The same approach was applied to the clinical model (pathology Gleason score) with the result that a cut-off of a Gleason score <=6 would lead to the stratification of only 57 men towards e.g. active surveillance instead of e.g. surgery. The progression-free survival in this patient cohort would be close to 90% but on the cost that a lot more men with non-progressive disease would be stratified to e.g. surgery; the use of PCPI_18 stratifies more than twice as many men to AS compared to pathology Gleason score while the risk profile is very comparable (85% vs. 90% progression-free survival, respectively). The stratification power can be even further increased by a combination model of PCPI_18 and the pathology Gleason. Using that model 146 men (compared to 134) would be stratified to AS with a progression-free survival probability over 10 years of around 87%.

The cut-off analysis for the shown patient cohort was performed for the total cohort (545 patients), a sub-cohort with pathology Gleason scores>=7 (482 patients), as well as another sub-cohort with pathology Gleason scores>=8 (211 patients).

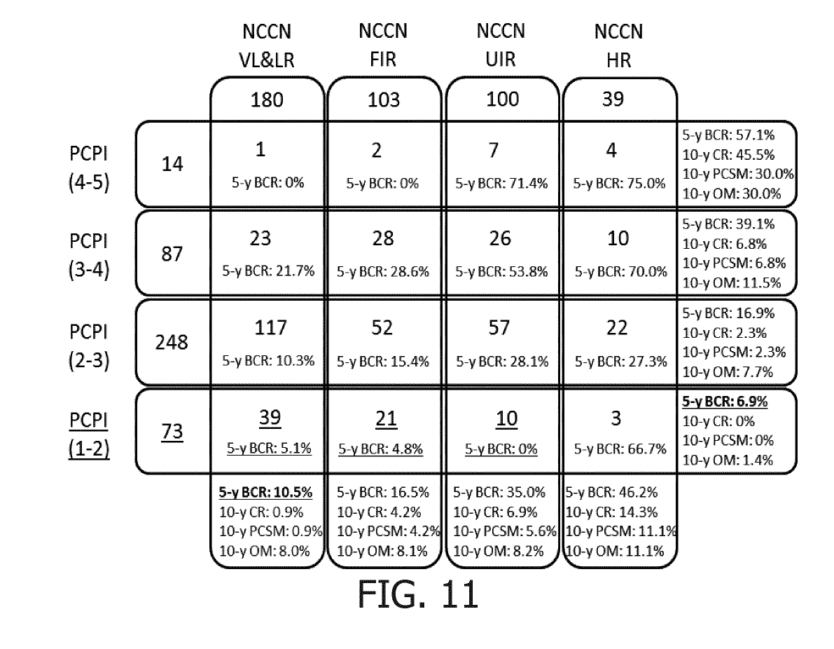

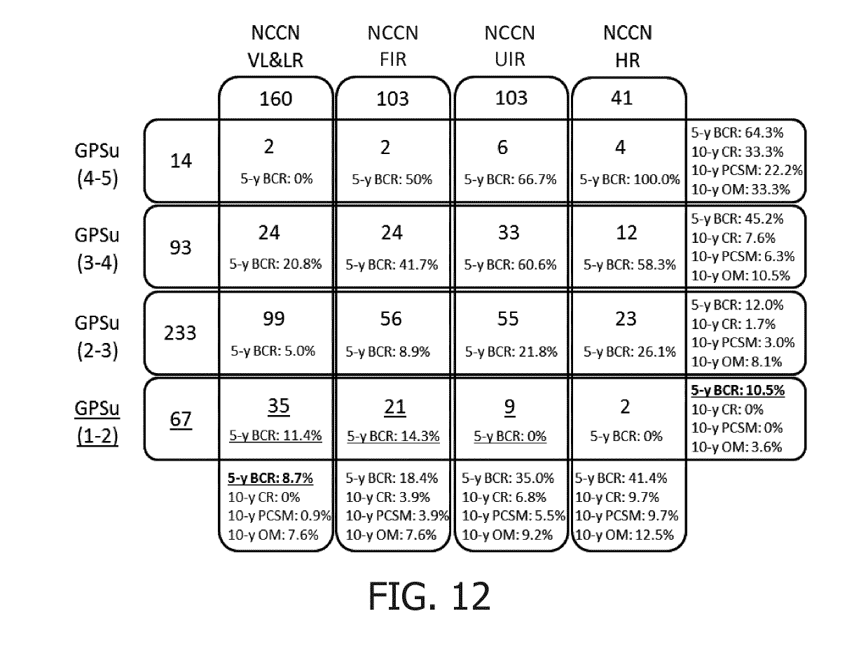

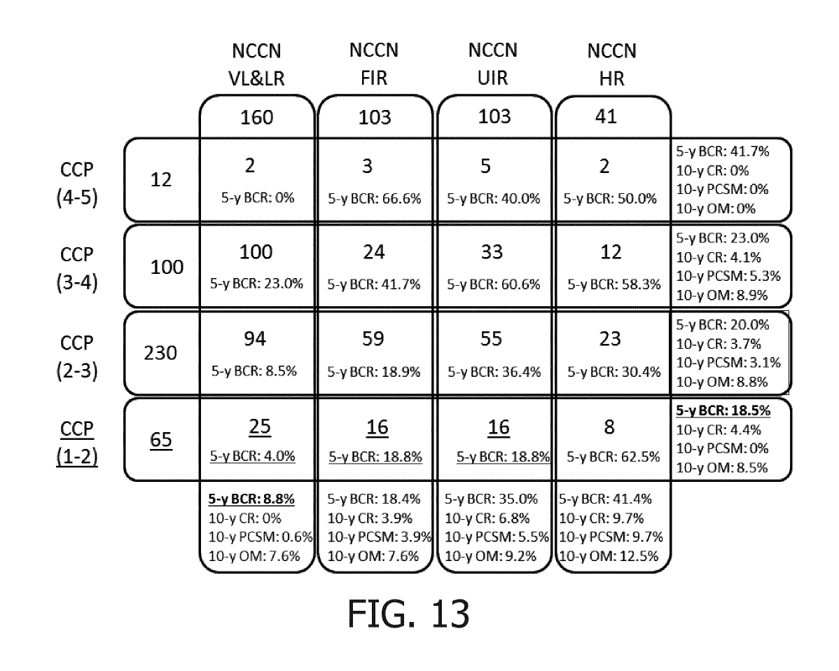

FIGS. 11, 12 and 13 refer to an analysis obtained from a study, which has been performed on approx. 500 patients with longitudinal follow up comparing the performance of the PCPI_19 (FIG. 11), the GPSu score, which is derived from RNAseq data analyzing the expression of 12 cancer related genes and 5 reference genes and was calculated following the method as far as it can be inferred from the article “Analytical validation of the Oncotype DX prostate cancer assay—a clinical RT-PCR assay optimized for prostate needle biopsies” by Dejan Knezevic et al. in BMC Genomics (2013), 14: 690 (FIG. 12) and the CCP score derived from the expression levels of 31 genes and calculated as far as can be inferred from the article “Prognostic value of an RNA expression signature derived from cell cycle proliferation genes for recurrence and death from prostate cancer: A retrospective study in two cohorts” by Jack Cuzick et al. in Lancet Oncol. (2011); 12(3): 245-255) (FIG. 13).

For better comparison, the GPSu and CCP score have been scaled to a range between 1 to 5.

The FIGS. 11, 12 and 13 show different recurrence risks (see below for description) over 5 or 10 years according to different patient groups. On the x-axis, patient groups have been defined by the NCCN risk groups; VL&LR=very low and low risk; FIR=favorable intermediate risk; UIR=unfavorable intermediate risk; HR=high risk. The NCCN are commonly used US guidelines in oncology (https://www.nccn.org/professionals/physician_gls/f_guidelines.asp). On the y-axis the patient groups have been defined by 4 categories of the different scores: score 1-2; 2-3; 3-4, and 4-5. In the 4×4 grid, patients have been selected for any of the cells of the grid according to their NCCN clinical risk characteristics and according to the score they have for PCPI, GPSu or CCP. The different risk according to clinical risk group was calculated (the line at the bottom) or according to score category (the column on the right hand side of the table).

Within the cells only the 5-year BCR recurrence risk is depicted for reasons of clarity.

The risk descriptions are:

5-y BCR—biochemical recurrence over 5 years

10-y CR—clinical recurrence to metastases over 10 years

10-y PCSM—prostate cancer specific mortality over 10 years

10-y OM—overall mortality (due to all reasons) over 10 years

The numbers underlined in FIGS. 11, 12 and 13 indicate areas of improved performance of PCPI over the other signatures. The patients that would fall into these categories are potential patients for active surveillance compared to active treatment clinical decisions and should therefore have preferably as little recurrence risk as possible. The PCPI outperforms the other signatures in these areas.

1. A method comprising:
determining a gene expression level for the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, to obtain a subject expression profile for a subject, and further comprising:
classifying the subject as having a good prognosis or a poor prognosis of prostate cancer based on the subject expression profile, wherein the good prognosis predicts an increased likelihood of survival within a predetermined period after initial diagnosis and/or no progression of disease after primary treatment, and the poor prognosis predicts an aggressive disease, a decreased likelihood of survival, and an increased likelihood of biochemical recurrence, clinical recurrence, and/or the presence of local or distant metastases, within a predetermined period after initial diagnosis; and/or
classifying the subject as having or not having a predisposition of prostate cancer that is susceptible to disease progression based on the subject expression profile, wherein the predisposition predicts an aggressive disease, decreased likelihood of survival, and an increased likelihood of biochemical recurrence, clinical recurrence, and/or the presence of local or distant metastases, within a predetermined period after initial diagnosis.

2. (canceled)

3. A method comprising:
classifying a subject as having a good prognosis or a poor prognosis based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, and wherein a good prognosis predicts an increased likelihood of survival within a predetermined period after initial diagnosis and/or no progression of disease after primary treatment, and poor prognosis predicts an aggressive disease, a decreased likelihood of survival, and an increased likelihood of biochemical recurrence, clinical recurrence, and/or the presence of local or distant metastases, within a predetermined period after initial diagnosis; and/or
classifying a subject as having or not having a predisposition of prostate cancer that is susceptible to disease progression based on a subject expression profile for the subject, wherein the subject expression profile includes the gene expression level of the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7, wherein the predisposition predicts an aggressive disease, decreased likelihood of survival, an increased likelihood of biochemical recurrence, clinical recurrence, and/or the presence of local or distant metastases, within a predetermined period after initial diagnosis.

4. The method of claim 1, wherein the subject expression profile is converted to a prostate cancer progression index.

5. The method of claim 4, wherein the prostate cancer progression index is calculated according to the following equation:
PCPI=(GEV_av_GOI_up)−(GEV_av_GOI_down),
wherein GEV av GOI up is an average gene expression value of up-regulated genes, and wherein GEV_av_GOI_down is an average gene expression value of down-regulated genes, wherein the values are determined on the basis of normalized gene expression data per each subject sample.

6. The method of claim 1, wherein the expression level of the set of the signature genes is normalized to the expression of a reference gene, preferably wherein the reference gene is a housekeeping gene, and more preferably wherein the housekeeping gene is TBP, HPRT1, ACTB, RPLP0, PUM1, POLR2A or B2M.

7. (canceled)

8. The method of claim 3,
wherein the subject is classified as having a good prognosis if the prostate cancer progression index is below a selected threshold or as having a poor prognosis if the prostate cancer progression index is above the selected threshold; and/or
wherein the subject is classified as having a predisposition of prostate cancer that is susceptible to disease progression if the prostate cancer progression index is above a selected threshold or as not having a predisposition of prostate cancer that is susceptible to disease progression if the prostate cancer progression index is below the selected threshold.

9. The method of claim 8, further comprising
stratifying the subject for the risk of aggressive disease versus non-aggressive disease according to the prognosis determined or the predisposition determined; and/or
providing a suitable cancer treatment to the subject in need thereof according to the prognosis determined or the predisposition determined.

10. The method of claim 3, wherein the poor prognosis or the predisposition of prostate cancer that is susceptible to disease progression is indicative of eligibility of the subject to be treated with any one or more of the treatments selected from the group consisting of prostate surgery, prostate removal, chemotherapy, radiotherapy, brachytherapy, limited or extended lymph node dissection, hormonal therapy.

11. Use of a product comprising:
primers and/or probes for determining the gene expression level for the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7;
optionally further comprising primers and/or probes for determining the expression levels of a set of genes listed in either FIG. 1 or 2 other than the above-mentioned genes; and/or
optionally further comprising primers and/or probes for determining the gene expression level of a reference gene, preferably wherein the reference gene is a housekeeping gene, and more preferably wherein the housekeeping gene is TBP, HPRT1, ACTB, RPLP0, PUM1, POLR2A or B2M,
for establishing a prognosis for a patient diagnosed with prostate cancer, for stratification of a patient diagnosed with prostate cancer, or for the determination of predisposition for aggressive or indolent prostate cancer in a patient having prostate cancer.

12. Use according to claim 11, wherein the product is a PCR kit, a RNA-sequencing kit, or a microarray or a microarray kit.

13. (canceled)

14. A computer program product, comprising computer readable code stored on a computer readable medium or downloadable from a communications network, which, when run on a computer, implement one or more steps of claim 1.

15. A system comprising the computer program product of claim 14 and a product comprising:
primers and/or probes for determining the gene expression level for the signature genes: AZGP1, FBLN1, ILK, KRT15, MEIS2, MYBPC1, PAGE4, SRD5A2, COL1A1, COL3A1, COL5A2, INHBA, THBS2, VCAN, BGN, BIRC5, and DYRK2, and optionally PDE4 isoforms comprising PDE4D5 and/or PDE4D7;
optionally further comprising primers and/or probes for determining the expression levels of a set of genes listed in either FIG. 1 or 2 other than the above-mentioned genes; and/or
optionally further comprising primers and/or probes for determining the gene expression level of a reference gene, preferably wherein the reference gene is a housekeeping gene, and more preferably wherein the housekeeping gene is TBP, HPRT1, ACTB, RPLP0, PUM1, POLR2A or B2M.

16. The system of claim 15, wherein the product is a PCR kit, a RNA-sequencing kit, or a microarray or a microarray kit.
